SEQUENCE LISTING (3 sequences)

SEQ ID NO: 1 - DNA, 29 bp, Streptomyces venezuelae
cacccccgaggggaatgaggtgtggctgg

SEQ ID NO: 2 - DNA, 29 bp, Streptomyces venezuelae
ccagccacacctcattcccctcgggggtg

SEQ ID NO: 3 - protein, 747 aa, Streptomyces venezuelae
MRRTQQGTTASPPVLDLGALGQDFAADPYPTYARLRAEGPAHRVRTPEGNEVWLVVGYDRARAVLADPRFSKDWRNSTTPLTEAEAALNHNMLESDPPRHTRLRKLVAREFTMRRVELLRPRVQEIVDGLVDAMLAAPDGRADLMESLAWPLPITVISELLGVPEPDRAAFRVWTDAFVFPDDPAQAQTAMAEMSGYLSRLIDSKRGQDGEDLLSALVRTSDEDGSRLTSEELLGMAHILLVAGHETTVNLIANGMYALLSHPDQLAALRADMTLLDGAVEEMLRYEGPVESATYRFPVEPVDLDGTVIPAGDTVLVVLADAHRTPERFPDPHRFDIRRDTAGHLAFGHGIHFCIGAPLARLEARIAVRALLERCPDLALDVSPGELVWYPNPMIRGLKALPIRWRRGREAGRRTGEFVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGPGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

(19) United States
(12) Patent Application Publication
(10) Pub. No.: US 2012/0190635 A1
(43) Pub. Date: Jul. 26, 2012

(54) SELECTIVE OXIDATION OF C-H BONDS OF MODIFIED SUBSTRATES BY P450 MONOOXYGENASE

(75) Inventors: Shengying Li, Ann Arbor, MI (US); David H. Sherman, Ann Arbor, MI (US); John Montgomery, Ann Arbor, MI (US); Mani R. Chaulagain, Evansville, IN (US); Allison R. Knauff, Ann Arbor, MI (US)

(73) Assignee: THE REGENTS OF THE UNIVERSITY OF MICHIGAN, Ann Arbor, MI (US)

(21) Appl. No.: 13/388,308

(22) PCT Filed: Sep. 27, 2010
(86) PCT No.: PCT/US10/50335

§ 371 Date: Apr. 10, 2012

Related U.S. Application Data
(60) Provisional application No. 61/245,958, filed on Sep. 25, 2009.

(51) Int. Cl. A61K 31/704; C12P 19/44; C12P 19/56; C12P 33/20; C12P 19/62; C07H 15/203; C07H 15/24; C07H 17/08; C07J 41/00; C07H 15/20; C07C 217/52; A61K 31/7048; A61K 31/7034; A61K 31/13; A61K 31/133; A61P 31/04; A61K 31/7028; C07H 15/10; C12N 9/02

(52) U.S. Cl. 514/26; 435/189; 435/74; 435/78; 435/53; 435/76; 536/16.8; 536/7.1; 536/5; 564/454; 564/457; 514/28; 514/34; 514/25; 514/659; 514/660

(57) ABSTRACT

The present invention provides regio- and stereoselective oxidation of unactivated C—H bonds using an engineered mutant cytochrome P450 monooxygenase and an engineered substrate.

### CROSS-REFERENCE TO RELATED APPLICATIONS

The benefit is claimed of U.S. provisional application No. 61/245,958, filed Sep. 25, 2009, the disclosure of which is incorporated by reference in its entirety herein.

### STATEMENT OF GOVERNMENT SUPPORT

This invention was made with U.S. Government support under Research Grant No. GM078553 and GM57014 awarded by the National Institutes of Health. The U.S. Government has certain rights in the invention.

### FIELD OF THE INVENTION

The present invention relates to regio- and stereoselective oxidation of unactivated C—H bonds of modified substrates using P450, including wild type P450, engineered and mutant P450 monooxygenase, such as PikC (PikC_{D50N}—RhFRED).

### BACKGROUND

Macrolides are a group of drugs (typically antibiotics) whose bioactivity stems from the presence of a macrolide ring, a large macrocyclic lactone ring, to which one or more deoxy sugars may be attached. The lactone rings are usually 14, 15 or 16-membered rings. Macrolides are a large family of polyketide natural products which include, but ae not limited to, erythromycin, spiramycin, FK506, and avermectin (Katz et al., Ann. Rev. Microbial. 47: 875-912, 1993; Hopwood, Chem. Rev. 97: 2465-2497, 1997). Macrolides are classified as polyketides which are secondary metabolites from bacteria, fungi, plants, and animals that are biosynthesized by the polymerization of acetyl and propionyl subunits. Polyketides are the building blocks for a broad range of natural products. A natural product is a chemical compound or substance produced by a living organism found in nature that usually has a pharmacological or biological activity for use in pharmaceutical drug discovery and drug design. Natural products are considered as such even if they are prepared by total synthesis. Not all natural products can be fully synthesized and many natural products have very complex structures that are too difficult and expensive to synthesize on an industrial scale.

Polyketides are structurally a very diverse family of natural products with diverse biological activities and pharmacological properties. Polyketide antibiotics, antifungals, cytostatics, anticholesterolemics, antiparasitics, coccidiostatics, animal growth promoters and natural insecticides are in commercial use.

Cytochrome P450 enzymes (P450s) are highly attractive biocatalysts due to their ability to catalyze a variety of regio- and stereo-specific oxidation reactions of complex organic compounds. These reactions occur under mild conditions by taking advantage of the two-electron activated dioxygen that is often challenging in organic synthesis (Ortiz de Montellano, P. R. Cytochrome P450: Structure, Mechanism and Biochemistry, 2nd ed. Ortiz de Montellano P. R., Ed. 1995, New York: Plenum Press. p473; Guengerich, F. P. Chem. Res. Toxicol. 2001, 14, 611). To activate molecular oxygen, redox partners are required to sequentially transfer two reducing equivalents from NAD(P)H to P450 (Hannemann, F. B. A., Ewen K. M., Bernhardt, R. Biochim. Biophys. Acta. 2007. 1770, 330). Classically, there are two major redox partner systems, including an FAD-containing reductase with a small iron-sulfur (Fe₂S₂) redoxin for most bacterial and mitochondrial P450s (Class I), and a single FAD/FMN-containing flavoprotein for eukaryotic microsomal P450s (Class II) (Lewis, D. F. V., Hlavica P. Biochim. Biophys. Acta 2000, 1460, 353; Munro, A. W., Girvan H. M., McLean K. J. Nat. Prod. Rep. 2007, 24, 585). The inherent requirement of cytochrome P450s for separate protein redox partners significantly limits their application in biotechnology.

The discovery of the first self-sufficient P450_{BM3}, which is naturally fused to a eukaryotic-like reductase represents an effective solution to this limitation (Ruettinger, R. T., Fulco, A. J. J. Biol. Chem. 1981, 256, 5728; Otey, C. R., Bandara, G., Lalonde, J., Takahashi, K., Arnold, F. H. Biotechnol. Bioeng. 2005, 93, 494). Self-sufficient cytochrome P450s have an activity that is independent of separate redox partners ferredoxin and ferredoxin reductase. The fusion nature of this enzyme dramatically improves electron transfer efficiency and coupling with the oxidative process, enabling it to be the most efficient P450 enzyme characterized to date (Munro, A. W., Leys D. G., McLean, K. J., Marshall, K. R., Ost, T. W. B., Daff, S., Miles, C. S., Chapman, S. K., Lysek, D. A., Moser, C. C., Page, C. C., Dutton, P. L. Trends Biochem. Sci. 2002, 27, 250). Based upon the self-sufficiency of this naturally fused enzyme, a number of engineered proteins of diverse eukaryotic P450s bearing a reductase domain from P450_{BM3} have been generated with in vitro activities (Fairhead, M., Giannini, S., Gillam, E. M. J.; Gilardi, G. J. Biol. Inorg. Chem. 2005, 10, 842; Dodhia, V. R., Fantuzzi, A., Gilardi, G. J. Biol. Inorg. Chem. 2006, 11, 903). This work provides ready access to the great catalytic versatility of the membrane-bound eukaryotic P450s. In contrast, the biosynthetic P450s (Class I) lack such a universal reductase that is used to engineer diverse self-sufficient P450s for either functional identification or potential industrial application.

Recently, a new class of self-sufficient cytochrome P450s exemplified by P450_{RhF} from Rhodococcus sp. NCIMB 9784 was discovered to be naturally fused to a novel FMN/Fe₂S₂ containing reductase partner (De Mot, R., Parret, A. H. A. Trends Microbiol. 2002, 502; Roberts, G. A., Grogan, G., Greter, A., Flitsch, S. L., Turner, N. J. J. Bacteriol. 2002, 184, 3898). Although the physiological function of P450_{RhF} remains unknown, its reductase domain (RhFRED), which is similar to the phthalate family of dioxygenase reductases, is capable of transferring electrons from NADPH to the heme domain of the monooxygenase, supporting 7-ethoxycoumarin dealkylation activity (Roberts, G. A., celik, A., Hunter, D. J. B., Ost, T. W. B., White, J. H., Chapman, S. K., Turner, N. J., Flitsch, S. L. J. Biol. Chem. 2003, 48914; Hunter, D. J. B., Roberts, G. A., Ost, T. W. B., White, J. H., Müller, S., Turner, N. J., Flitsch, S. L., Chapman, S. K. FEBS Lett. 2005, 579, 2215). Moreover, recent reports from Misawa et al. demonstrated that this reductase domain could be used to reconstitute the catalytic activities of various Class I P450s in vivo through expression of corresponding genes fused to RhFRED in Escherichia coli cells (Kubota, M., Nodate, M., Yasumoto-Hirose, M., Uchiyama, T., Kagami, O., Shizuri, Y., Misawa, N. Biosci. Biotechnol. Biochem. 2005, 69, 2421; Nodate, M., Kubota, M., Misawa, N. Appl. Microbiol. Biotechnol. 2006, 71, 455). This observation suggests that RhFRED might be developed into a generally effective redox partner for biosynthetic bacterial P450s. However, the lack of corresponding in vitro data could not unambiguously exclude in trans involvement of additional cellular redox partners.

Among various reactions catalyzed by P450 enzymes, the regio- and stereoselective oxidation of an unactivated sp³ C—H bond represents a central challenge in organic and synthetic chemistry (Dick A. R. and Sanford M. S. Transition metal catalyzed oxidative functionalization of C—H bonds. Tetrahedron 2006, 62:2439-2463; Crabtree R. H. Alkane C—H activation and functionalization with homogeneous transition metal catalysts: a century of progress—a new millennium in prospect. J. Chem. Soc., 2001 Dalton Trans.: 2437-2450; Bergman R. G. Organometallic chemistry: C—H activation. Nature 2007 446:391-393; Labinger J. A. and Bercaw J. E. Understanding and exploiting C—H bond activation. Nature 2002 417:507-514; Godula K., Sames, D. C—H bond functionalization in complex organic synthesis. Science 2006 312:67-72.). Considerable effort has been devoted to identifying biological catalysts or simpler mimics that function by mechanisms typically involving a metal oxo reactive site (Jr Que L. and Tolman W. Biologically inspired oxidation catalysis. Nature 2008 445:333-340). Alternatively, transition metal complexes have been identified for C—H bond oxidations that proceed through mechanisms completely distinct from biological systems. A key challenge in developing useful C—H oxidation procedures is the control of site-selectivity among similar C—H bonds. Successful approaches have typically involved either relying on the inherent reactivity differences of various C—H bonds based on steric and electronic considerations (Chen M. S. and White C. M. A predictably selective aliphatic C—H oxidation reaction for complex molecule synthesis. Science 2007 318:783-787; Brodsky B. H. and Du Bois J. Oxaziridine-mediated catalytic hydroxylation of unactivated 3° C—H bonds using hydrogen peroxide J. Am. Chem. Soc. 2005 127:15391-15393; Wender P. A., Hilinski M. K. and Mayweg A. V. Late-stage intermolecular CH activation for lead diversification: a highly chemoselective oxyfunctionalization of the C-9 position of potent bryostatin analogues. Org. Lett. 2005 7:79-82; Lee S, and Fuchs P. L. Chemospecific chromium[VI] catalyzed oxidation of C—H bonds at −40° C. J. Am. Chem. Soc. 2002 124:13978-13979; Chen H., Schlecht S., Semple T. C., and Hartwig J. F. Thermal, catalytic, regiospecific functionalization of alkanes. Science 2000 287:1995-1997; Cook B. R., Reinert T. J., and Suslick K. S. Shape-selective alkane hydroxylation by metalloporphyrin catalysts. J. Am. Chem. Soc. 1986 108:7281-7286), or the incorporation of directing groups that orient the catalyst active site towards a specific C—H bond (Desai L. V., Hull K. L., and Sanford M. S. Palladium-catalyzed oxygenation of unactivated sp3 C—H bonds J. Am. Chem. Soc. 2004 126:9542-9543; Dick A. R., Hull K. L., and Sanford M. S. A highly selective catalytic method for the oxidative functionalization of C—H bonds. J. Am. Chem. Soc. 2004 126:2300-2301; Dangel B. D., Johnson J. A. and Sames D. Selective functionalization of amino acids in Water: A synthetic method via catalytic C—H bond activation. J. Am. Chem. Soc. 2001 123:8149-8150). The selective oxidation of a C—H bond that is neither inherently more reactive than alternate sites nor positioned adjacent to a directing group poses the most difficult application in site-selective C—H bond functionalization.

Recent reports have shown that supramolecular organometallic assemblies provide some success in this challenge for synthetic chemistry (Das S., Incarvito C. D., Crabtree R. H. and Brudvig G. W. Molecular recognition in the selective oxygenation of saturated C—H bonds by a dimanganese catalyst. Science 2006 312:1941-1943; Das S., Brudvig G. W. and Crabtree R. H. High turnover remote catalytic oxygenation of alkyl groups: how steric exclusion of unbound substrate contributes to high molecular recognition selectivity J. Am. Chem. Soc. 2008 130:1628-1637; Breslow R., et al. Remote oxidation of steroids by photolysis of attached benzophenone groups J. Am. Chem. Soc. 1973 95:3251-3262; Yang J., Gabriele B., Belvedere S., Huang Y. and Breslow R. Catalytic oxidations of steroid substrates by artificial cytochrome P-450 enzymes. J. Org. Chem. 2002 67:5057-5067; Grieco P. A. and Stuk T. L. Remote oxidation of unactivated carbon-hydrogen bonds in steroids via oxometalloporphinates J. Am. Chem. Soc. 1990 112:7799-7801; Groves J. T. and Neumann R. Regioselective oxidation catalysis in synthetic phospholipid vesicles. Membrane-spanning steroidal metalloporphyrins J. Am. Chem. Soc. 1989 111:2900-2909). Alternatively, biological catalysts may provide unique potential to selectively oxidize bonds that are chemically similar, yet remote from directing influences. Thus, the potential role of biosynthetic CYP450 monooxygenases remains unclear despite their fundamental dependence on substrate-enzyme complementarity, which might limit their application in synthetic chemistry (Urlacher V. B. and Eiben S. Cytochrome P450 monooxygenases: perspectives for synthetic application. Trends Biotechnol. 2006 24:324-330). A number of previous efforts have sought to overcome this limitation by employing protein engineering strategies, including scanning chimeragenesis (Landwehr M., Carbone M., Otey C. R., Li Y. and Arnold F. H. Diversification of catalytic function in a synthetic family of chimeric cytochrome P450s. Chem. Biol. 2007 14:269-278; Sieber V., Martinez C. A. and Arnold F. H. Libraries of hybrid proteins from distantly related sequences. Nat. Biotechnol. 2001 19:456-460) and directed-evolution (Arnold F. H. Fancy footwork in the sequence-space shuffle. Nat. Biotechnol. 2006 24:328-330; Bloom J. D. et al. Evolving strategies for enzyme engineering. Curr. Opin. Struc. Biol. 2005 15:447-452; Kumar S., Chen C. S., Waxman D. J. and Halpert J. R. Directed Evolution of Mammalian Cytochrome P450 2B1. J. Biol. Chem. 2005 280:19569-19575; Bloom J. D. and Arnold F. H. In the light of directed evolution: pathway of adaptive protein evolution. Proc. Natl. Acad. Sci. USA 2009 106:9995-10000) to generate non-natural cytochrome P450s (e.g., P450BM3) with novel substrate specificities and abilities to selectively oxidize target substrates.

Thus there exists a need in the art to develop materials and methods for improved site-selective activation of C—H bonds to form, e.g., hydroxylated or epoxidized products.

### SUMMARY OF THE INVENTION

The present invention provides a method of hydroxylating a substrate comprising contacting the substrate with a cytochrome P450 enzyme to form a hydroxylated or epoxidized product, wherein the substrate comprises a desosaminyl, 1,2-diol-3-dimethylaminocyclohexane, 1,2-diol-3-methylaminocyclohexane, or N,N-dimethylaminopropylether moiety.

In various aspects, the cytochrome P450 enzyme is a bacterial biosynthetic cytochrome P450, a mitochondrial cytochrome P450 or a eukaryotic cytochrome P450. In certain embodiments, the cytochrome P450 enzyme is PikC, EryF, MycG, TylI, TylHI, or TamI.

In certain aspects, the cytochrome P450 enzyme is mutated. In specific embodiments, the mutated cytochrome P450 is PikC, EryF, MycG, TylI, TylHI, or TamI. In one embodiment, the mutated cytochrome P450 is PikC. In another embodiment, the mutated PikC is PikC_{D50N}.

In various embodiments, the P450 enzyme is fused to a heterologous reductase domain. In one embodiment, the reductase domain is RhFRED from Rhodococcus sp. NCIMB 9784.

In certain aspects, the substrate is a polyketide, a macrolide, a cycloalkane, an aromatic compound, a heteroaromatic compound, or a steroid. In other aspects, the desosaminyl, 1,2-diol-3-dimethylaminocyclohexane, 1,2-diol-3-methylaminocyclohexane, or N,N-dimethylaminopropylether moiety is covalently attached to the substrate through an acetal bond, an ester bond, an ether bond, a ketal bond, a peptide bond, a carbon-carbon bond, a hemi-acetal bond, or a hemi-ketal bond.

In some aspects, the desosaminyl, 1,2-diol-3-dimethylaminocyclohexane, 1,2-diol-3-methylaminocyclohexane, or N,N-dimethylaminopropylether moiety is covalently attached to the substrate through an acetal bond. In other aspects, the substrate is covalently attached to the desosaminyl, 1,2-diol-3-dimethylaminocyclohexane, 1,2-diol-3-methylaminocyclohexane, or N,N-dimethylaminopropylether moiety at a C-1 oxygen. In still other aspects, desosaminyl, 1,2-diol-3-dimethylaminocyclohexane, 1,2-diol-3-methylaminocyclohexane, or N,N-dimethylaminopropylether moiety is removed from the hydroxylated or epoxidized product. In some aspects, the desosaminyl moiety is covalently attached to the substrate through an acetal bond. In some aspects, the 1,2-diol-3-dimethylaminocyclohexane or 1,2-diol-3-methylaminocyclohexane moiety is covalently attached to the substrate through an ether bond. In various aspects, the N,N-dimethylaminopropylether moiety is covalently attached to the substrate through an ether bond.

The invention also contemplates a P450 substrate modified with a desosaminyl, 1,2-diol-3-dimethylaminocyclohexane, 1,2-diol-3-methylaminocyclohexane, or N,N-dimethylaminopropylether moiety, wherein P450 does not act or is less active on the substrate without the desosaminyl, 1,2-diol-3-dimethylaminocyclohexane, 1,2-diol-3-methylaminocyclohexane, or N,N-dimethylaminopropylether moiety. In various embodiments, the substrate comprises a polyketide, a macrolide, a cycloalkane, an aromatic compound, a heteroaromatic compound, or a steroid. In other embodiments, the substrate is modified with desosaminyl moiety at a C-1 position in the desosamine. In still other embodiments, the desosaminyl moiety is covalently attached to the substrate through an acetal bond, an ester bond, an ether bond, a ketal bond, a peptide bond, a carbon-carbon bond, a hemi-acetal bond, or a hemi-ketal bond. In still other embodiments, the 1,2-diol-3-dimethylaminocyclohexane, 1,2-diol-3-methylaminocyclohexane, or N,N-dimethylaminopropylether is covalently attached to the substrate through an ether bond, a ketal bond, a peptide bond, a carbon-carbon bond, a hemi-acetal bond, or a hemi-ketal bond.

The invention also contemplates a chimeric protein comprising a mutant cytochrome P450 and a heterologous reductase domain wherein the chimeric protein has self-sufficiency and increased catalytic efficiency compared to native P450 monooxygenases, said chimeric protein having the activity of catalyzing C—H bond oxidation of a substrate modified with a desosaminyl, 1,2-diol-3-dimethylaminocyclohexane, 1,2-diol-3-methylaminocyclohexane, or N,N-dimethylaminopropylether moiety.

In various embodiments, the substrate is a polyketide, a cycloalkane, an aromatic molecule, a heteroaromatic molecule, an alkyne, or a steroidyl molecule. In other embodiments, the C—H oxidization comprises oxidation of one or more primary, secondary or tertiary carbon atoms of said substrate. Oxidation can be hydroxylation or epoxidation.

In various aspects, the mutant cytochrome P450 is a mutant bacterial biosynthetic cytochrome P450, a mutant mitochondrial cytochrome P450 or a mutant eukaryotic cytochrome P450. In other aspects, the mutant cytochrome P450 is PikC_{D50N}. In still other aspects, the mutant cytochrome P450 is a mutant PikC, a mutant EryF, a mutant MycG, a mutant TylI, a mutant TylHI, or a mutant TamI.

In another embodiment, the heterologous reductase domain is RhFRED from Rhodococcus sp. NCIMB 9784.

The chimeric protein is further provided in a purified state.

In yet another aspect, provided herein are compounds having a formula of

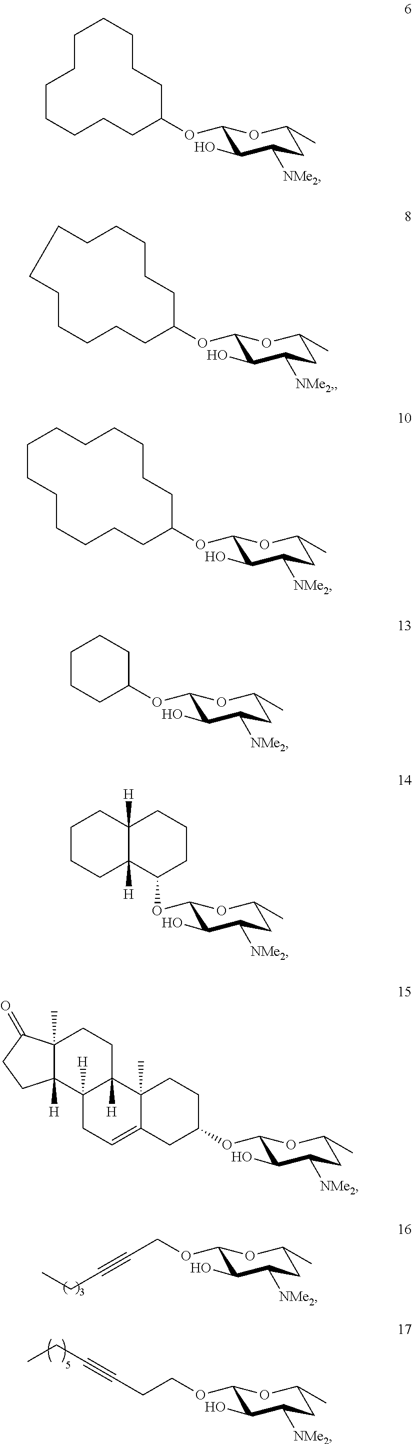

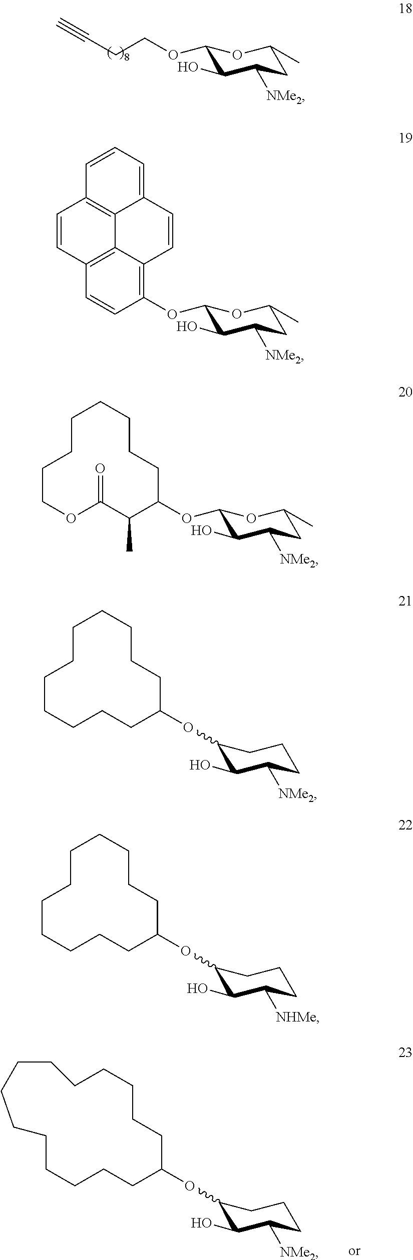

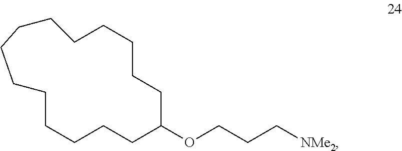

or salt thereof. In some cases, these compounds, or their hydroxylated or epoxidized derivative, have antibacterial activity. Thus, further provided is a method of inhibiting bacterial activity in a cell comprising contacting the cell with a compound as disclosed herein or a substrate having a desosaminyl moiety. In some cases, the substrate comprises a polyketide, a macrolide, a cycloalkane, an aromatic compound, a heteroaromatic compound, or a steroid. In various cases, the desosaminyl, 1,2-diol-3-dimethylaminocyclohexane, 1,2-diol-3-methylaminocyclohexane, or N,N-dimethylaminopropylether is covalently attached to the substrate through an acetal bond, an ester bond, an ether bond, a ketal bond, a peptide bond, a carbon-carbon bond, a hemi-acetal bond, or a hemi-ketal bond.

### BRIEF DESCRIPTION OF THE FIGURES

FIG. 1. Schematic strategy of substrate engineering.

FIG. 2. Major physiological reactions catalyzed by PikC.

FIG. 3. LC-MS analysis of PikC_{D50N}—RhFRED catalyzed reactions using different cyclized carbolides as substrates. (Ion count chromatograms are shown) (A) Desosaminyl cyclododecane 6 reaction, 7b and 7f correspond to two diastereomers generated by C7 hydroxylation, 7a, 7c, 7e, and 7g correspond to four diastereomers generated by C6/C8 hydroxylation; (B) Desosaminyl cyclotridecane 8 reaction, 9a, 9d and 9f correspond to diastereomers arising from C6/C9 hydroxylation, 9b and 9c correspond to diastereomers originated from C7/C8 or C6/C9 hydroxylation. The number of products that peak 9c contains is undetermined due to product overlap; (C) Desosaminyl cyclotetradecane 10 reaction; (D) Desosaminyl cyclopentadecane 12 reaction. The details of product assignment for 6 and 8 based on correlation with synthesized authentic standards regarding retention time and co-injection confirmation are shown in Supporting Information.

FIG. 4. Multiple binding modes of desosaminyl cycloalkanes. Orientations of 6 in the active site of PikC_{D50N} (A) in chain A and (B) in chain B, and orientations of 8 in the active site of PikC_{D50N} (C) in chain A and (D) in chain B, as defined by the fragments of the electron density map (gray mesh) contoured at 0.8 Å of 6 are shown. In (B), 6 is docked in the flipped-over orientations allowing hydroxylation on the both sides of the ring. In (C) and (D), 8 is in flipped over orientations. Heme is shown in orange. Oxygen atoms are in red, nitrogen in blue, iron in orange. Atoms of the cycloalkane ring are labeled in red. Distances are in Angstroms. Images are generated using PYMOL.

FIG. 5. Desosaminyl cycloalkane binding sites. (A) Stereo view of the PikC_{D50N} binding site with the three superimposed 6 conformers highlighted in gray (chain A), pink and cyan (chain B) surrounded by the chain A amino acid side chains within 5 Å plus E85 (green) is shown. E246 of chain B is highlighted in ice blue. To increase clarity, V242 was omitted from the drawing. (B) Stereo view of the PikC_{D50N} binding site with two superimposed 8 conformers highlighted in pink (chain A) and cyan (chain B) surrounded by the chain A amino acid side chains within 5 Å plus E85 is shown. F178 of chain B is highlighted in ice blue. Heme is shown in orange. Oxygen atoms are in red, nitrogen in blue, iron in orange. Atoms of the cycloalkane ring are labeled in red. Distances between tertiary amine and carboxylic groups are in Angstroms. Lower-case a in the residue label indicates that alternative conformations are shown.

FIG. 6. General synthetic strategy for glycosylation of diverse alcohols with desosamine (Chen, H., Yamase, H., Murakami, K., Chang, C.-w., Zhao, L., Zhao, Z. and Liu, H.-w. 2002, Biochemistry 41, 9165-9183. 2. Anzai, Y., Li, S., Chaulagain, M. R., Kinoshita, K., Kato, F., Montgomery, J. and Sherman, D. H. 2008 Chem. Biol. 15, 950-959).

FIG. 7. Michaelis-Menten curve of PikC_{D50N}—RhFRED using YC-17 1 as substrate.

FIG. 8. Mass spectra of 6 (top panel) and its hydroxylated products 7a-g (middle panel). The bottom panel shows the MS-MS pattern of one of products. Notably, all desosaminyl derivatives in this study show the same MS-MS pattern.

FIG. 9. Structural determination of mono-hydroxylated products of 6 through LC-MS comparison of synthetic authentic standards to 7a-g regarding retention times. (A) Product profile of PikC_{D50N}—RhFRED reaction using 6 as substrate; (B) Authentic standard containing a pair of C6/C8 hydroxylated diastereomers; (C) Authentic standard containing the other pair of C6/C8 hydroxylated diastereomers; (D) Authentic standard containing the pair of C7 hydroxylated diastereomers. The products assignment was further confirmed by co-injections.

FIG. 10. Structural determination of mono-hydroxylated products of 8 through LC-MS comparison of synthetic authentic standards to 9a-f regarding retention times. (A) Product profile of PikC_{D50N}—RhFRED reaction using 8 as substrate; (B) Authentic standard containing a pair of C6/C9 hydroxylated diastereomers; (C) Authentic standard containing the other pair of C6/C9 hydroxylated diastereomers; (D) Authentic standard containing a pair of C7/C8 hydroxylated diastereomers; (E) Authentic standard containing the other pair of C7/C8 hydroxylated diastereomers. The products assignment was further confirmed by co-injections. Some diastereomers are not distinguishable due to identical retention times (e.g. traces D and E) Notably, to get a better separation of similar diastereomers, an optimized LC condition other than the one described in the main text was employed: mobile phase, 10% B for 3 min, 10-80% B over 25 min, 80-100% B over 1 min, 100% B for 5 min, 100-10% solvent B over 2 min, 10% solvent B for 15 min. All other conditions remained the same.

FIG. 11. (A) Product profile of PikC_{D50N}—RhFRED reaction using linear desosaminyl derivative 17 as substrate. (B) Amplified product profile to visualize the hydroxylated products in small amounts. Mass spectra of 17 (C) and its hydroxylated products (D).

FIG. 12. (A) Product profile of PikC_{D50N}—RhFRED reaction using linear desosaminyl derivative 18 as substrate. (B) Amplified product profile to visualize the hydroxylated products in small amounts. Mass spectra of 18 (C) and its hydroxylated products (D).

FIG. 13. (A) Product profile of PikC_{D50N}—RhFRED reaction using linear desosaminyl derivative 18 as substrate. (B) Authentic standard containing a pair of C9 hydroxylated diastereomers. The products assignment was further confirmed by co-injection. The right panel shows the amplified chromatograms to visualize the hydroxylated products in small amounts.

FIG. 14. (A) Product profile of PikC_{D50N}—RhFRED reaction using aromatic desosaminyl pyrene 19 as substrate. Mass spectra of 19 (B) and its C8 hydroxylated product 20 (C).

FIG. 15. (A) Product profile of PikC_{D50N}—RhFRED reaction using 3-(cyclopentadecyloxy)-N,N-dimethylpropan-1-amine 24 as substrate. (B) Product profile of PikC_{D50N}—RhFRED reaction using compound 20 as substrate.

FIG. 16. (A) Product profile of PikC_{D50N}—RhFRED reaction using compound 22 as substrate. (B) Product profile of PikC_{D50N}—RhFRED reaction using compound 23 as substrate. The lower panel shows the amplified product profile.

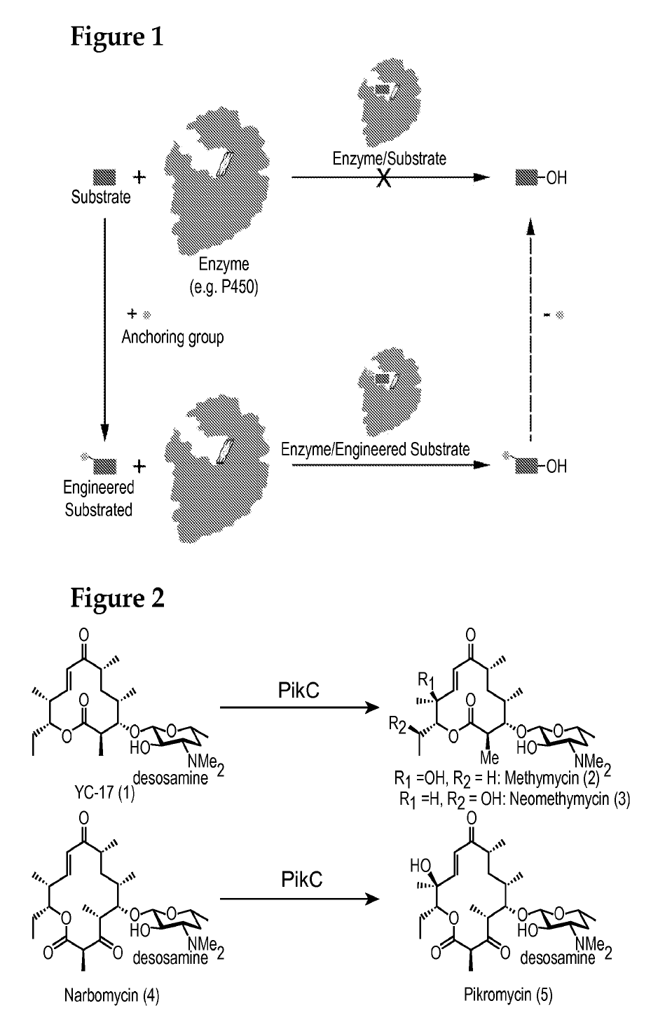

### DETAILED DESCRIPTION OF THE INVENTION

Herein is provided the first in vitro implementation of substrate engineering for selective C—H bond oxidations. Regio- and stereoselective oxidation of an unactivated C—H bond is a central challenge in organic chemistry. Considerable effort has been devoted to identifying transition metal complexes, biological catalysts, or simplified mimics, but limited success has been achieved. Cytochrome P450 monooxygenases are involved in diverse types of regio- and stereoselective oxidations and represent a promising biocatalyst to address this challenge. The application of this class of enzymes is particularly significant if their substrate spectra can be broadened, selectivity controlled and reactions catalyzed in the absence of expensive heterologous redox partners.

In one aspect of the invention, methods are provided wherein substrate engineering is employed for selective C—H bond oxidations using an optimized form of the macrolide P450 monooxygenase. In one embodiment, the P450 monooxygenase is PikC (PikC_{D50N}—RhFRED). In another embodiment, methods are provided wherein a series of carbocyclic rings linked to a desosamine substrate is effectively hydroxylated in a regioselective manner. In one aspect, the substrate is a carbocyclic ring linked to the desosamine glycoside (referred to as a carbolide). Analysis of a series of high-resolution enzyme-substrate co-crystal structures provides significant new insights into the function of the aminosugar-derived anchoring group for control of reaction site selectivity. Unexpected biological activity of a select number of these carbolide systems reveals their potential as a new class of antibiotics.

### Cytochrome P450s

The superfamily of cytochrome P450 enzymes is involved in diverse oxidative processes including xenobiotic catabolism, steroid synthesis, and biosynthetic tailoring of diverse natural products. These reactions occur under mild conditions by taking advantage of the two-electron activated dioxygen that is often challenging in organic synthesis (Ortiz de Montellano, P. R. Cytochrome P450: Structure, Mechanism and Biochemistry, 2nd ed. Ortiz de Montellano P. R., Ed. 1995, New York: Plenum Press. p473; Guengerich, F. P. Chem. Res. Toxicol. 2001, 14, 611). To activate molecular oxygen, redox partners are required to sequentially transfer two reducing equivalents from NAD(P)H to P450 (Hannemann, F. B. A., Ewen K. M., Bernhardt, R. Biochim. Biophys. Acta. 2007. 1770, 330). Classically, there are two major redox partner systems, including an FAD-containing reductase with a small iron-sulfur (Fe₂S₂) redoxin for most bacterial and mitochondrial P450s (Class I), and a single FAD/FMN-containing flavoprotein for eukaryotic microsomal P450s (Class II) (Lewis, D. F. V., Hlavica P. Biochim. Biophys. Acta 2000, 1460, 353; Munro, A. W., Girvan H. M., McLean K. J. Nat. Prod. Rep. 2007, 24, 585). The inherent requirement of cytochrome P450s for separate protein redox partners significantly limits their application in biotechnology.

Cytochrome P450-mediated electron transport is responsible for oxidative metabolism of both endogenous compounds, which include but are not limited to, fatty acids, steroids, prostaglandins, and exogenous compounds ranging from therapeutic drugs, antibiotics, and environmental toxicants to carcinogens. Vitamins are also potential products envisioned by the invention. For example, both vitamin D₂ and vitamin D₃ are metabolized into prohormones by one or more enzymes located in the liver. The involved enzymes are mitochondrial and microsomal cytochrome P450 (CYP) isoforms, including CYP27A1, CYP2R1, CYP3A4, CYP2J3 and possibly others. These enzymes metabolize vitamin D₂ into two prohormones known as 25-hydroxyvitamin D₂ and 24(S)-hydroxyvitamin D₂, and Vitamin D₃ into a prohormone known as 25-hydroxyvitamin D₃.

The term “cytochrome P450” or simply “P450” is used to encompass any cytochrome P450 enzyme. As such, the P450s encompassed by the present invention include prokayortic, eukaryotic, bacterial and mitochondrial enzymes. Cytochrome P450 (often abbreviated as CYP, P450, and infrequently CYP450) is a very large and diverse superfamily of hemoproteins which form part of multicomponent electron transfer chains, called P450-containing systems. Known cytochrome P450s amenable to the invention include the CYP1 family (CYP1A1; CYP1A2; CYP1B1), the CYP2 family (CYP2A6; CYP2A7, CYP2B6, CYP2A13; CYP2B6; CYP2C8; CYP2C9; CYP2C18, CYP2C19; CYP2D6; CYP2E1; CYP2F1; CYP2J2; CYP2R1; CYP2S1; CYP2U1, CYP2W1), the CYP3 family (CYP3A4; CYP3A5; CYP3A7; CYP3A43), the CYP4 family (CYP4A11; CYP4A22; CYP4B1; CYP4F2, CYP4F3, CYP4F8, CYP4F11, CYP4F12, CYP4V2, CYP4Z1), CYP5A1, CYP7A1, CYP7B1, CYP8A1, CYP8B1, CYP11A1, CYP11B1, CYP11B2, CYP17A1, CYP19A1, CYP20A1, CYP21A2, CYP24A1, CYP26A1, CYP26B1, CYP26C1, CYP27A1, CYP27B1, CYP27C1, CYP39A1, CYP46A1, and CYP51A1.

“An “analogue,” “analog,” “derivative,” or “variant” refers to a polypeptide substantially similar in structure and having the same biological activity, albeit in certain instances to a differing degree, to a naturally-occurring molecule, e.g., the P450. Analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence, (ii) insertion or addition of one or more amino acids at one or more termini (typically an “addition” analog) of the polypeptide and/or one or more internal regions (typically an “insertion” analog) of the naturally-occurring polypeptide sequence or (iii) substitution of one or more amino acids for other amino acids in the naturally-occurring polypeptide sequence.

A “fragment” of a polypeptide refers to any portion of the polypeptide smaller than the full-length polypeptide or protein expression product. Fragments are, in one aspect, deletion analogs of the full-length polypeptide wherein one or more amino acid residues have been removed from the amino terminus and/or the carboxy terminus of the full-length polypeptide. Methods for preparation of deletion analogs are routine in the art. See, e.g., Sambrook et al. (2001) Molecular Cloning: A Laboratory Guide, 3^{rd} ed., Cold Spring Harbor Press, the disclosure of which is incorporated herein by reference in its entirety.

In one aspect, an analog exhibits about 70% sequence similarity but less than 100% sequence similarity with a given compound, e.g., a peptide. Such analogs or derivatives are, in one aspect, comprised of non-naturally occurring amino acid residues, including by way of example and not limitation, homoarginine, ornithine, penicillamine, and norvaline, as well as naturally occurring amino acid residues. Such analogs or derivatives are, in another aspect, composed of one or a plurality of D-amino acid residues, or contain non-peptide interlinkages between two or more amino acid residues. The term “derived from” as used herein refers to a polypeptide or peptide sequence that is a modification (including amino acid substitution or deletion) of a wild-type or naturally-occurring polypeptide or peptide sequence and has one or more amino acid substitutions, additions or deletions, such that the derivative sequence shares about 70% but less than 100% sequence similarity to the wild-type or naturally-occurring sequence. In one embodiment, the derivative may be a fragment of a polypeptide, wherein the fragment is substantially homologous (i.e., at least 70%, at least 75%, at least 80%, at least 85%, at least 90%, or at least 95% homologous) over a length of at least 5, 10, 15, 20, 25, 30, 35, 40, 45 or 50 amino acids of the wild-type polypeptide.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection. One example of a useful algorithm is PILEUP, which uses a simplification of the progressive alignment method of Feng & Doolittle, J. Mol. Evol. 35:351-360 (1987) and is similar to the method described by Higgins & Sharp, CABIOS 5:151-153 (1989). Another algorithm useful for generating multiple alignments of sequences is Clustal W (Thompson, et al., Nucleic Acids Research 22: 4673-4680 (1994)). An example of algorithm that is suitable for determining percent sequence identity and sequence similarity is the BLAST algorithm (Altschul, et al., J. Mol. Biol. 215:403-410 (1990); Henikoff & Henikoff, Proc. Natl. Acad. Sci. USA 89:10915 (1989); Karlin & Altschul, Proc. Natl. Acad. Sci. USA 90:5873-5787 (1993)). Software for performing BLAST analyses is publicly available through the National Center for Biotechnology Information.

Substitutions are conservative or non-conservative based on the physico-chemical or functional relatedness of the amino acid that is being replaced and the amino acid replacing it. Substitutions of this type are well known in the art. Alternatively, the invention embraces substitutions that are also non-conservative.

### PikC

The PikC cytochrome P450 is involved in the pikromycin biosynthetic pathway of Streptomyces venezuelae (Xue, Y., Zhao, L., Liu, H.-w., Sherman, D. H. Proc. Natl. Acad. Sci. U.S.A. 1998, 95, 12111). PikC catalyzes the final hydroxylation step toward both the 12-membered ring macrolactone YC-17 and the 14-membered ring macrolactone narbomycin to produce methymycin/neomethymycin and pikromycin as major products (Xue, Y., Wilson, D., Zhao, L., Liu, H.-w., Sherman, D. H. Chem. Biol. 1998, 5, 661; Lee, S. K., Park, J. W., Kim, J. W., Jung, W. S., Park, S. R., Choi, C. Y., Kim, E. S., Ahn, J. S., Sherman, D. H., Yoon, Y. J. J. Nat. Prod. 2006, 69, 847).

Recent analysis of x-ray co-crystal structures of PikC (Sherman D. H., et al. The structural basis for substrate anchoring, active site selectivity, and product formation by P450 PikC from Streptomyces venezuelae. J. Biol. Chem. 2006 281:26289-26297; Li S., Ouellet H., Sherman D. H. and Podust L. M. Analysis of transient and catalytic desosamine-binding pockets in cytochrome P-450 PikC from Streptomyces venezuelae. J. Biol. Chem. 2009 284:5723-5730) involving endogenous substrates revealed that the macrolactone ring contacts the active site residues entirely via nonspecific hydrophobic interactions, likely account for the tolerance of PikC toward the variant macrolactone ring size and functionalization. In contrast, the desosamine sugar employs two distinct binding pockets and anchors the substrate through a number of hydrogen bonds and ionic interactions, in particular, a unique salt bridge between the protonated dimethylamino group of desosamine and a glutamate residue, either Glu94 or Glu85 in the B/C loop region. Based on these previously recognized molecular interactions that specify substrate binding affinity and orientation in the binding pocket, desosamine could be an effective anchoring group to direct positioning of various unnatural molecules in the active site of PikC for selective C—H bond hydroxylations.

### EryF

Cytochrome P450 EryF, isolated from the actinomycete bacterium Saccharopolyspora erythraea, is responsible for the biosynthesis of the antibiotic erythromycin by C6-hydroxylation of the macrolide 6-deoxyerythronolide B (DEB). Following hydroxylation, the 6-DEB macrolide ring is further elaborated to erythromycin A (Lambalot, R. H., Cane, D. E. Biochemistry 1995, 34, 1858; Andersen J. F., Tatsuta K., Gunji H., Ishiyama T., Hutchinson C. R. Biochemistry 1993, 32, 1905-1913; Andersen, J. F., Hutchinson, R. C. J. Bacteriol. 1992, 174, 725; Ogura, H., Nishida, C. R., Hoch, U. R., Perera, R., Dawson, J. H., Ortiz de Montellano, P. R. Biochemistry 2004, 43, 14712). When RhFRED was fused to another prototype biosynthetic P450 EryF, a more active self-sufficient biocatalyst was obtained once again (Li, S., Podust, L. M., and Sherman, D. H. J. Am. Chem. Soc. 2007, 129, 12940-12941).

### MycG and TamI

MycG, one P450 monooxygenase from the mycinamicin biosynthetic pathway is able to either hydroxylate or epoxidize mycinamicin IV (M-IV) leading to mycinamicin V (M-V) or mycinamicin I (M-I), respectively. M-V can be further epoxidized by MycG resulting in final product mycinamicin II (M-II) (Anzai, Y., Li, S. et al. Chem. Biol. 2008, 15, 950-959). TamI was recently characterized as another versatile P450 enzyme, which is capable of catalyzing multiple hydroxylation and epoxidation reactions, thus leading to remarkable post-PKS—NRPS structural diversification in tirandamycin biosynthesis (Carlson J. C. et al. ChemBioChem 2010, 11, 564-572).

### TylI and TylHI

TylI, the P450 monooxygenase from the tylosin biosynthetic pathway of Streptomyces fradiae, catalyzes the C20 hydroxylation of O-mecaminosyltylactone giving rise to 20-dihydro-23-deoxy-O-mycaminosyltylactone. It is also responsible for oxidation of 20-dihydro-23-deoxy-O-mycaminosyltylactone, leading to formation of 23-deoxy-β-mycaminosyltylactone (Baltz, R. H. and Seno, E. T. Ann. Rev. Microbiol. 1988, 42, 547-574). TylHI is the second P450 enzyme in the same tylosin pathway that hydroxylates 23-deoxy-O-mycaminosyltylactone leading to O-mycaminosyltylactone (Baltz, R. H. and Seno, E. T. Ann. Rev. Microbiol. 1988, 42, 547-574).

### P450 Reductase Domain

In its simplest form, the P450 monooxygenase system consists of NAD(P)H dependent cytochrome P450 reductase (CPR; NAD(P)H-ferrihemoprotein reductase) and one of many cytochrome P450 enzymes listed above. Both CPR and cytochrome P450 are integral membrane proteins, and CPR is one of only two known mammalian enzymes containing both FMN (flavin mononucleotide or riboflavin-5′-phosphate) and FAD (flavin adenine dinucleotide) as prosthetic groups, the other being various isoforms of nitric-oxide synthase (NOS). Other physiological electron acceptors of CPR include microsomal heme oxygenase (Schacter, B. A., Nelson, E. B., Marver, H. S. & Masters, B. S. S. 1972, J. Biol. Chem. 247, 3601-3607) and cytochrome b5 (Enoch, H. G. & Strittmatter, P. 1979, J. Biol. Chem. 254, 8976-8981) and, although nonphysiological, CPR is capable of transferring reducing equivalents to cytochrome c (Horecker, B. L. 1950, J. Biol. Chem. 183, 593-605).

Consequently, the term “reductase domain” encompasses any reductase function (i.e., electron donation). Reductase domains include bacterial, microsomal, mitochondrial, fungal, eukaryotic, pant and animal reductase domains. In certain embodiments, reductase domains contemplated by the invention include, but are not limited to, NAD(P)H-dependent ferrihemoprotein reductase, ferredoxin, ferredoxin reductase, flavodoxin, flavodoxin reductase, putidaredoxin, and putidaredoxin reductase.

The term “self-sufficiency” means the activity of cytochrome P450 is independent of separate redox partners, such as and without limitation, ferredoxin and ferredoxin reductase. Self-sufficient P450 is re-oxidized by the fused reductase, so it is independently re-activated and there is no need for a separate redox partner.

### P450 Chimeric Fusion Proteins

As previously described and incorporated herein by reference (U.S. Patent Publication No. 2009/0081758 and Li S., Podust L. M. and Sherman D. H. Engineering and analysis of a self-sufficient biosynthetic cytochrome P450 PikC fused to the RhFRED reductase domain. J. Am. Chem. Soc. 2007 129:12940-12941), single component bacterial biosynthetic cytochrome P450s are fused to a heterologous reductase domain. These chimeric fusion proteins demonstrate high catalytic efficiency. P450s typically need two redox partners, ferredoxin and ferredoxin reductase, in order to catalyze natural product synthesis reactions through electron transfer. The fusion nature of these chimeric proteins dramatically improves electron transfer efficiency and coupling with the oxidative process, i.e., hydroxylation and/or epoxidation, enabling the chimeric (or fusion) proteins to be more efficient, highly catalytic and more cost-effective P450 enzymes compared to native cytochrome P450s.

“Mutant” “engineered” or “variant” P450 chimeric fusion proteins encompass mutated cytochrome P450s fused to a heterologous reductase domain. In one embodiment, the previously prepared PikC template (described in U.S. Patent Application 2009/0081758 and Li et al., supra), and site-directed mutagenesis are used to substitute D50 of PikC to Asn (N) or Ala (A). Elimination of the surface-exposed negative charge at Asp⁵° (and replacement in this instance with asparagine) results in significantly enhanced catalytic activity. Mutation of any cytochrome P450 is contemplated by the present invention. The mutated cytochrome P450s are then fused to a heterologous reductase domain as described above in order to catalyze synthesis reactions through electron transfer.

### Purification of Mutant P450 Chimeric Fusion Proteins

In one embodiment of the invention, the purification of the mutant P450 chimeric fusion enzyme is accomplished using previously developed procedures with minor modifications (Xue, Y., Wilson, D., Zhao, L., Liu, H.-w., Sherman, D. H. Chem. Biol. 1998, 5, 661-667; Li, S., Podust, L. M., and Sherman, D. H. J. Am. Chem. Soc. 2007, 129, 12940-12941; Anzai, Y., Li, S., Chaulagain, M. R., Kinoshita, K., Kato, F., Montgomery, J., and Sherman D. H. Chem. Biol. 2008, 15, 950-959). Cells transformed with desired plasmids are grown and harvested by centrifugation. The cell pellet is resuspended and lysis is accomplished using a sonic dismembrator. The insoluble material is then separated by centrifugation and the soluble fraction is collected and loaded onto a Ni-NTA column, whereby the contaminants are removed by wash buffer. The elution buffer is added onto the column, and eluted protein fraction is concentrated and desalted by buffer exchange with a PD-10 column.

In another aspect, purification of the mutant P450 chimeric fusion enzyme is accomplished using various purification methods known in the art including, but not limited to, affinity chromatography, (Yasukochi Y and Masters BSS 1976, J. Biol. Chem. 251: 5337-5344), nickel-nitrotriacetic acid column chromatography (Helvig, C. Koener, J. F., Unnithan, G. C. and Feyereisen, R. 2004, PNAS 101:4024-4029), aminooctyl Sepharose 4B column chromatography (Matsunaga et al., Drug Metab Dispos. 1998, (10):1045-7), phenyl-Sepharose hydrophobic interaction column (Funhoff, E. G. et al. J. Bacteriol. 2006 188(14): 5220-5227), size exclusion high performance liquid chromatography (Nakhgevany, R. et al. Biochem. Biophys. Res. Comm. 1996, (222) 406-409) cation exchange chromatography, anion exchange chromatography (Yasukochi, Y., Okita, R. T. & Masters, B. S. S. 1980, Arch. Biochem. Biophys. 202, 491-498), hydroxylapatite chromatography and DEAE-Sepharose CL-6B column chromatography (Sasaki, M. et al., Applied and Environmental Microbiology 2005, (71) 8024-8030). Also contemplated by the invention are purification procedures such as differential centrifugation, solubilization of CHAPS, protein precipitation by PEG precipitation and DE-32 column chromatography (Cai-Hong Yu and Xi-Wu Gao Insect Science 2005, (12) 313-317).

The term “purified” as used herein refers to a protein composition that has been isolated under conditions that reduce or eliminate the presence of unrelated materials, i.e., contaminants, including native materials from which the material is obtained. For example, a purified enzyme is preferably substantially free of host cell or culture components, including tissue culture or egg proteins, non-specific pathogens, and the like. In one embodiment, purified material substantially free of contaminants is at least 50% pure; at least 60%, at least 70%, at least 80%, at least 90%, at least 95% or even at least 99% pure. Purity is evaluated by chromatography, gel electrophoresis, immunoassay, composition analysis, biological assay, and other methods known in the art.

### Modified Substrates

In one aspect, the methods disclosed herein are used to oxidize a substrate using a P450 enzyme as described herein. The substrate is any molecule having a desosaminyl moiety attached. The modified substrate is a substrate for P450, wherein when the desosaminyl moiety is not present, the substrate is either not active as a P450 substrate or is less active as a P450 substrate, compared to when the desosaminyl moiety The desosaminyl, 1,2-diol-3-dimethylaminocyclohexane, 1,2-diol-3-methylaminocyclohexane, or N,N-dimethylaminopropylether moiety in various aspects, is attached to the substrate through a covalent bond, such as and without limitation an acetal, an ester, an ether, a ketal, a peptide, a carbon carbon, a hemi-acetal, or a hemi-ketal bond. The desosaminyl moiety has a structure:

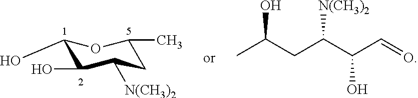

The 1,2-diol-3-dimethylaminocyclohexane moiety has a structure:

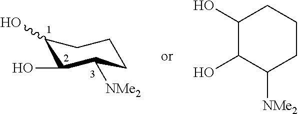

The 1,2-diol-3-methylaminocyclohexane moiety has a structure:

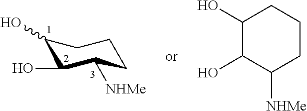

The N,N-dimethylaminopropylether moiety has a structure:

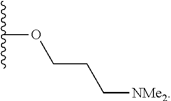

The substrate, in one aspect, is attached to any site on the desosaminyl moiety. In some cases, the substrate is attached to the desosaminyl moiety at the C-1 position and through an acetal bond. The substrate in some aspects is attached to the 1,2-diol-3-dimethylaminocyclohexane, 1,2-diol-3-methylaminocyclohexane, or N,N-dimethylaminopropylether moiety via an ether bond. Some non-limiting examples of substrates include polyketides, macrolides, cycloalkanes, aromatic compounds, heteroaryl compounds, and steroids.

The term “polyketide” encompasses a large class of diverse compounds that are characterized by more than two carbonyl groups connected by single intervening carbon atoms that are produced by bacteria, fungi, plants and animals. Polyketides include various substances having antibiotic, anticancer, cholesterol-lowering, or immunosuppressive effects. Polyketides include antibiotics such as tetracyclines, macrolides (such as erythromycin or pikromycin) and non-macrolides (such as tirandamycin or curacin), anticancer agents such as daunomycin, immunosuppressants such as FK506, tacrolimus, ascomycin, and rapamycin, and antifungals and antibacterials such as linearmycin A and lienomycin. Polyketides also include, but are not limited to, avermectin, nemadectin, candicidin, niddamycin, oleandomycin, narbomycin, rifamycin, and spiramycin. As will be appreciated by those skilled in the art, a wide variety of domains, modules, protein subunits as well as whole proteins are available from known polyketide synthase biosynthetic clusters that be used to make alterations in the biosynthesis of a polyketide and/or a macrolide, Polyketides biosynthesized from two-carbon units in a series of Claisen condensations in which the initially formed product at each condensation step is a β-keto ester (“ketide” hence the name “polyketide”). As used herein, the term “polyketide” refers to any naturally occurring or synthetic cyclic β-keto ester.

As used herein, the term “cycloalkanes” refers to a cyclic hydrocarbon, e.g., C₃₋₂₀ cyclic molecules, and preferably C₁₀₋₂₀ cyclic molecules. Cycloalkane and be saturated or partially unsaturated ring systems optionally substituted with, for example, one to three groups, independently selected from the group consisting of alkyl, alkyleneOH, C(O)NH2, NH2, oxo (═O), aryl, halo, and OH.

The term “macrolide” is a compound with a structure that contains a macrocyclic lactone ring to which one or more deoxy sugars may be attached. These compounds encompass any macrolide antibiotic, and include, for example and without limitation, a group of antibiotics produced by various strains of Streptomyces that have a complex macrocyclic structure. The lactone rings are usually 14, 15 or 16-membered rings. Macrolide antibiotics include, but are not limited to, erythromycin A, B and C, azithromycin, griesomycin, methymycin, narbomycin, neomethymycin, oleandomycin, pikromycin, plicacetin, carbomycin A and B, spriamycin I, II and III, leucomycin, mycinamicin, rapamycin, clarithromycin, FK506 (tacrolimus), FK520 (ascomycin or immunomycin), antascomicin, meridamycin, FK520, hyg, FK523, meridamycin, antascomicin, FK525, tsukubamycin, ivermectin, milbemycin D, soraphen A, vancomycin, teicoplanin, roxithromycin, josamycin, ristocetin, actinoidin, avoparcin, actaplanin, teichomycin and telithromycin. Synthetic macrolide genes may comprise one or more of angMIII, angMI, angB, angAI, angAII, angorf14, angorf4, tylMIII, tylMI, tylB, tylAI, tylAII, eryCVI, spnO, eryBVI, eryK, tyl, and eryG, eryCIII, tylMII, angMII, desVII, eryBV, spnP, midI, or the like.

As used herein, the term “aromatic compoud” refers to a monocyclic or polycyclic aromatic molecule, preferably a monocyclic or bicyclic aromatic group, e.g., phenyl or naphthyl. Unless otherwise indicated, an aryl group is unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Exemplary aryl groups include, but are not limited to, phenyl, naphthyl, fluorocenyl, biphenyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaromatic compound” refers to a monocyclic or bicyclic ring system containing one or two aromatic rings and containing at least one nitrogen, oxygen, or sulfur atom in an aromatic ring. Unless otherwise indicated, a heteroaryl group be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

As used herein, the term “steroid” refers to a hydrophobic compound characterized by a sterane core and additional functional groups. The core is a carbon structure of four fused rings: three cyclohexane rings and one cyclopentane ring. Steroids vary by the functional groups attached to these rings and the oxidation state of the rings. Non-limiting examples of steroids include clobetasol propionate, diflorasone acetate, fluocinonide, mometasone furancarboxylate, betamethasone dipropionate, betamethasone butyrate propionate, betamethasone valerate, difluprednate, budesonide, diflucortolone valerate, amcinonide, halcinonide, dexamethasone, dexamethasone propionate, dexamethasone valerate, dexamethasone acetate, hydrocortisone acetate, hydrocortisone butyrate, hydrocortisone butyrate propionate, deprodone propionate, prednisolone valerate acetate, fluocinolone acetonide, beclomethasone propionate, triamcinolone acetonide, flumethasone pivalate, alclomethasone propionate, clobethasone butyrate, prednisolone, beclomethasone propionate, fludroxycortide, and the like.

Modification of the substrate is readily performed using the knowledge of an organic chemist. Modification is at a functional group on the substrate, such as a hydroxyl, amino, sulfur, carboxylic acid, or the like.

In some embodiments, after C—H activation in the presence of the enzyme catalyst, the desosaminyl, 1,2-diol-3-dimethylaminocyclohexane, 1,2-diol-3-methylaminocyclohexane, or N,N-dimethylaminopropylether moiety of the hydroxylated or epoxideized substrate then is removed. Removal of the desosaminyl, 1,2-diol-3-dimethylaminocyclohexane, 1,2-diol-3-methylaminocyclohexane, or N,N-dimethylaminopropylether moiety will depend upon the type of covalent bond through which it is attached to the substrate. For example, an ester bond, acetal bond, ketal bond, peptide, hemi-acetal bond, or hemi-ketal bond is broken in a hydrolysis reaction. Known techniques for the removal of the desosaminyl, 1,2-diol-3-dimethylaminocyclohexane, 1,2-diol-3-methylaminocyclohexane, or N,N-dimethylaminopropylether moiety is used, similar to choice and removal of a protecting group, as extensively discussed in Wuts et al., Greene's Protective Groups in Organic Synthesis, 4^{th} ed., (Wiley Interscience: Hoboken, N.J.) 2007.

An enzyme is “regioselective” if the hydroxylation or epoxidation of the product that results from the enzymatic reaction is positioned at one position on the substrate over any other position. An enzyme that has an altered regioselectivity means that while the original P450 may have created a first amount of product A and a second amount of product B, the regioselective enzyme could produce a third amount of product A and a fourth amount of product B. The P450 enzyme used in the methods disclosed herein can be tailored to provide a selected regioselectivity of the substrate. Additionally or alternatively, the substrate can be tailored, e.g., through protecting groups or selection of ring size, to provide a desired regioselectivity.

An enzyme is “enantioselective” (or steroselective) if the hydroxylation or epoxidation reaction of the enzyme results in a high amount of one particular enantiomeric product compared to other possible enantiomeric products. An enzyme that has an altered enantioselectivity means that while the original P450 may have created a first amount of enantiomeric product A and a second amount of enantiomeric product B, the enantioselective enzyme could produce a third amount of enantiomeric product A and a fourth amount of enantiomeric product B. The P450 enzyme used in the methods disclosed herein can be tailored to provide a selected enantioselectivity of the substrate, through evaluation of different mutations or variants for the desired enantioselectivity. Additionally or alternatively, the substrate can be tailored, e.g., through protecting groups or selection of ring size, to provide a desired enantioselectivity.

### Antibiotic Compounds

Methods of producing an antibiotic using a mutant cytochrome P450 chimeric fusion protein are contemplated by the invention. In one embodiment, the method involves contacting a desosamine glycoside substrate with a mutant P450 chimeric fusion protein as described herein under conditions that permit the fusion protein to catalyze a reaction that produces an antibiotic. The mutant chimeric P450 fusion protein is either purified or non-purified. By way of example, antibiotic production includes, but is not limited to, pikromycin: A purified PikC_{D50N}—RhFRED cytochrome P450 fusion protein of the invention is used to cataylze the hydroxylation of narbomycin to produce the antibiotic pikromycin. Confirmation of transformation then be achieved by HPLC trace, mass spectrometry, UV spectrum analysis, or other methods known in the art.

Further provided herein are methods of inhibiting a bacterial infection comprising contacting a cell with a compound as disclosed herein. In some cases, the compound is a hydroxylated substrate, wherein prior to hydroxylation had a formula of

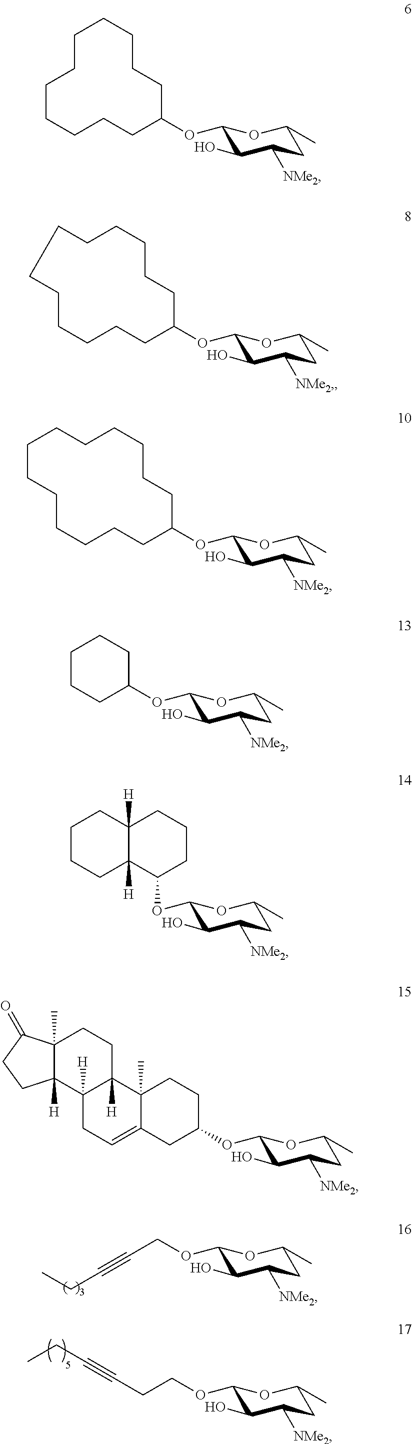

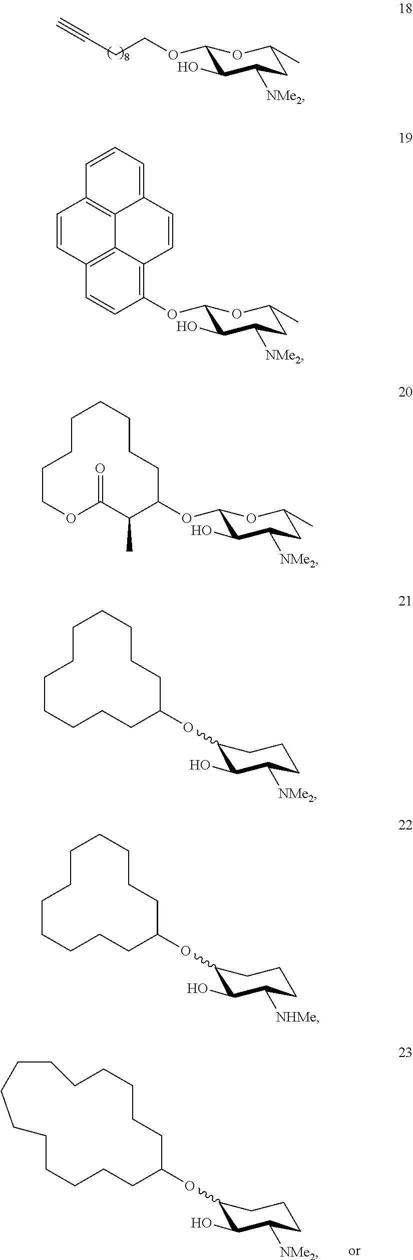

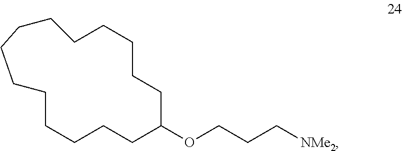

or salt thereof. In various cases, the compound has higher antibacterial activity than a derivative without the desosaminyl, 1,2-diol-3-dimethylaminocyclohexane, 1,2-diol-3-methylaminocyclohexane, or N,N-dimethylaminopropylether moiety and/or prior to hydroxylation or epoxidation.

The terms “therapeutically effective amount” and “prophylactically effective amount,” as used herein, refer to an amount of a compound sufficient to treat, ameliorate, or prevent the identified disease or condition, or to exhibit a detectable therapeutic, prophylactic, or inhibitory effect. The effect can be detected by, for example, an improvement in clinical condition, reduction in symptoms, or by any of the assays or clinical diagnostic tests described herein. The precise effective amount for a subject will depend upon the subject's body weight, size, and health; the nature and extent of the condition; and the therapeutic or combination of therapeutics selected for administration. Therapeutically and prophylactically effective amounts for a given situation can be determined by routine experimentation that is within the skill and judgment of the clinician.

Dosages of the therapeutic can alternately be administered as a dose measured in mg/kg. Contemplated mg/kg doses of the disclosed therapeutics include about 0.001 mg/kg to about 1000 mg/kg. Specific ranges of doses in mg/kg include about 0.1 mg/kg to about 500 mg/kg, about 0.5 mg/kg to about 200 mg/kg, about 1 mg/kg to about 100 mg/kg, about 2 mg/kg to about 50 mg/kg, and about 5 mg/kg to about 30 mg/kg.

As herein, the compounds described herein may be formulated in pharmaceutical compositions with a pharmaceutically acceptable excipient, carrier, or diluent. The compound or composition comprising the compound is administered by any route that permits treatment of the disease or condition. One route of administration is oral administration. Additionally, the compound or composition comprising the compound may be delivered to a patient using any standard route of administration, including parenterally, such as intravenously, intraperitoneally, intrapulmonary, subcutaneously or intramuscularly, intrathecally, topically, transdermally, rectally, orally, nasally or by inhalation. Slow release formulations may also be prepared from the agents described herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

Administration may take the form of single dose administration, or a compound as disclosed herein can be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition.

In an embodiment, the pharmaceutical compositions are formulated with one or more pharmaceutically acceptable excipient, such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, the pH is adjusted to a range from about pH 5.0 to about pH 8. More particularly, the pharmaceutical compositions may comprise a therapeutically or prophylactically effective amount of at least one compound as described herein, together with one or more pharmaceutically acceptable excipients. Optionally, the pharmaceutical compositions may comprise a combination of the compounds described herein, or may include a second active ingredient useful in the treatment or prevention of bacterial infection (e.g., anti-bacterial or anti-microbial agents.

Formulations, e.g., for parenteral or oral administration, are most typically solids, liquid solutions, emulsions or suspensions, while inhalable formulations for pulmonary administration are generally liquids or powders. A pharmaceutical composition can also be formulated as a lyophilized solid that is reconstituted with a physiologically compatible solvent prior to administration. Alternative pharmaceutical compositions may be formulated as syrups, creams, ointments, tablets, and the like.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

The pharmaceutical compositions described herein are formulated in any form suitable for an intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules wherein the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions may be formulated as suspensions comprising a compound of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents (e.g., sodium carboxymethylcellulose, methylcellulose, hydroxypropyl methylcellulose, sodium alginate, polyvinylpyrrolidone, gum tragacanth, gum acacia); dispersing or wetting agents (e.g., a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate)); and thickening agents (e.g., carbomer, beeswax, hard paraffin or cetyl alcohol). The suspensions may also contain one or more preservatives (e.g., acetic acid, methyl or n-propyl p-hydroxy-benzoate); one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using those suitable dispersing or wetting agents and suspending agents, including those mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

To obtain a stable water-soluble dose form of a pharmaceutical composition, a pharmaceutically acceptable salt of a compound described herein may be dissolved in an aqueous solution of an organic or inorganic acid, such as 0.3 M solution of succinic acid, or more preferably, citric acid. If a soluble salt form is not available, the compound may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to about 60% of the total volume. In one embodiment, the active compound is dissolved in DMSO and diluted with water.

The pharmaceutical composition may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In some embodiments, the compounds described herein may be formulated for oral administration in a lipid-based formulation suitable for low solubility compounds. Lipid-based formulations can generally enhance the oral bioavailability of such compounds.

As such, pharmaceutical compositions comprise a therapeutically or prophylactically effective amount of a compound described herein, together with at least one pharmaceutically acceptable excipient selected from the group consisting of medium chain fatty acids and propylene glycol esters thereof (e.g., propylene glycol esters of edible fatty acids, such as caprylic and capric fatty acids) and pharmaceutically acceptable surfactants, such as polyoxyl 40 hydrogenated castor oil.

In some embodiments, cyclodextrins may be added as aqueous solubility enhancers. Exemplary cyclodextrins include hydroxypropyl, hydroxyethyl, glucosyl, maltosyl and maltotriosyl derivatives of α-, β-, and γ-cyclodextrin. A specific cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the invention in the composition.

In some cases, provided herein are methods that further include identifying a subject having a bacterial infection and administering to the subject a compound as disclosed herein. In various cases, the methods provided herein are prophylactic methods, and a compound or composition as disclosed herein is administered prior to onset of a disorder. In certain cases, the method further comprises identifying a subject at risk of contracting a bacterial infection, and administering an effective amount of a compound as disclosed herein.

The invention will be more fully understood by reference to the following examples which detail exemplary embodiments of the invention. They should not, however, be construed as limiting the scope of the invention. All citations throughout the disclosure are hereby expressly incorporated by reference.

### EXAMPLES

### Example 1

### Engineered PikC_{D50N}—RhFRED is Capable of Hydroxylating Carbolides Effectively and Regioselectively

Using previously prepared pET28b-PikC—RhFRED as a template (Li S., Podust L. M. and Sherman D. H. Engineering and analysis of a self-sufficient biosynthetic cytochrome P450 PikC fused to the RhFRED reductase domain. J. Am. Chem. Soc. 2007 129:12940-12941), site-directed mutagenesis was performed by following the QuikChange (Stratagene) protocol. The primers for mutagenesis were: forward, 5′-CACCCCCGAGGGGAATGAGGTGTGGCTGG-3′ (SEQ ID NO: 1); reverse, 5′-CCAGCCACACCTCATTCCCCTCGGGGGTG-3′ (SEQ ID NO: 2). Protein expression and purification of PikC_{D50N}—RhFRED followed the procedure developed previously (Li et al., supra). SEQ ID NO: 3 is PIKC_{D50N}—RhFRED:

MRRTQQGTTASPPVLDLGALGQDFAADPYPTYARLRAEGPAHRVRTPEGN
EVWLVVGYDRARAVLADPRFSKDWRNSTTPLTEAEAALNHNMLESDPPRH
TRLRKLVAREFTMRRVELLRPRVQEIVDGLVDAMLAAPDGRADLMESLAW
PLPITVISELLGVPEPDRAAFRVWTDAFVFPDDPAQAQTAMAEMSGYLSR
LIDSKRGQDGEDLLSALVRTSDEDGSRLTSEELLGMAHILLVAGHETTVN
LIANGMYALLSHPDQLAALRADMTLLDGAVEEMLRYEGPVESATYRFPVE
PVDLDGTVIPAGDTVLVVLADAHRTPERFPDPHRFDIRRDTAGHLAFGHG
IHFCIGAPLARLEARIAVRALLERCPDLALDVSPGELVWYPNPMIRGLKA
LPIRWRRGREAGRRTGEFVLHRHQPVTIGEPAARAVSRTVTVERLDRIAD
DVLRLVLRDAGGKTLPTWTPGAHIDLDLGALSRQYSLCGAPDAPSYEIAV
HLDPESRGGSRYIHEQLEVGSPLRMRGPRNHFALDPGAEHYVFVAGGIGI
TPVLAMADHARARGWSYELHYCGRNRSGMAYLERVAGHGDRAALHVSEEG
TRIDLAALLAEPAPGVQIYACGPGRLLAGLEDASRNWPDGALHVEHFTSS
LAALDPDVEHAFDLELRDSGLTVRVEPTQTVLDALRANNIDVPSDCEEGL
CGSCEVAVLDGEVDHRDTVLTKAERAANRQMMTCCSRACGDRLALRL.

The reaction contains 40 nM of PikC—RhFRED-D50N with 3.5 μM spinach ferredoxin and 0.1 U/ml spinach ferredoxin-NADP+ reductase, 10˜160 μM substrate in 400 μl of desalting buffer (50 mM NaH2PO4, pH 7.3, 1 mM EDTA, 0.2 mM DTE, 10% glycerol). After pre-incubation at 30° C. for 5 min, the reaction was initiated by adding 4 μl of 50 mM NADPH and 100 μl aliquots were taken at 0 s, 20 s, and 40 s (or 0 s, 30 s, and 60 s when substrate concentrations are greater than 100 μM) to thoroughly mix with 100 μl of methanol for reaction termination. After centrifugation at 13,000 g for 15 min to pellet protein, the supernatants were analyzed by HPLC. The HPLC conditions were: Xbridge C18 5 μm 250 mm reverse-phase column, 20-80% solvent B (A: deionized water+0.1% trifluoroacetic acid, B: acetonitrile+0.1% trifluoroacetic acid) at 1.0 ml/min over 18 min, UV wavelength 226 nm. The initial velocity of substrate consumption was deduced from decreased area under the curve (AUC) of specific substrate peaks. Finally, the data from duplicated experiments were fit to Michaelis-Menten equation.

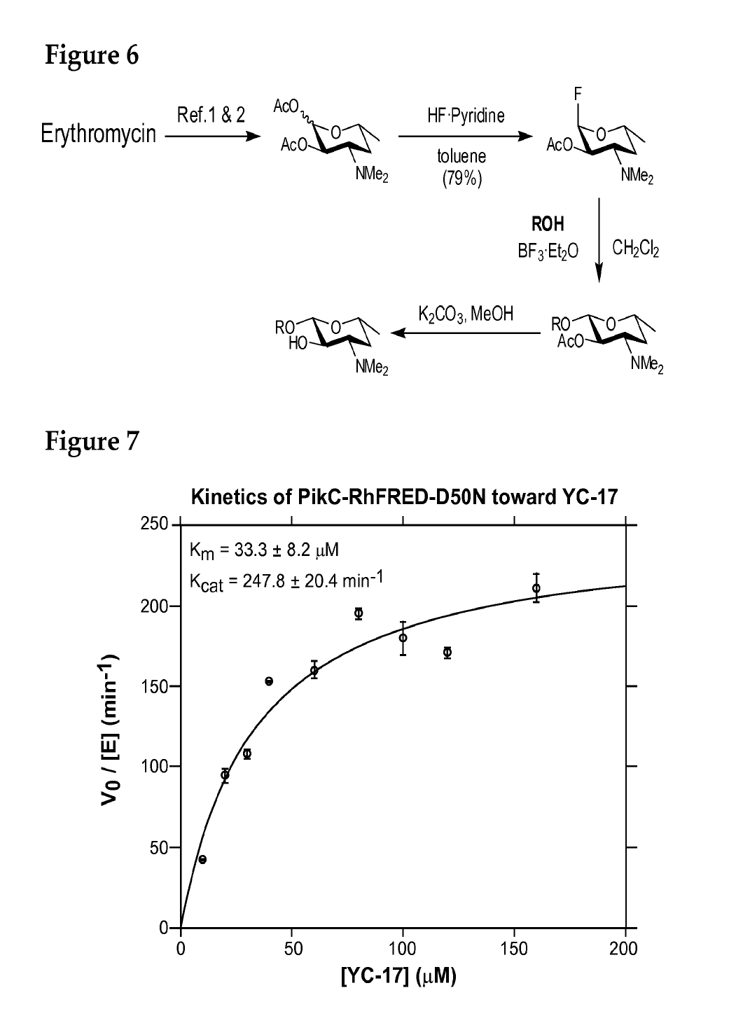

The unnatural cyclic carbolide substrate desosaminyl cyclododecane was synthesized to mimic the structure of the natural substrate YC-17 using a recently developed synthetic strategy (Anzai Y, et al. Functional analysis of MycG and MycCI, cytochrome P450 enzymes involved in biosyntheis of mycinamicin macrolide antibiotics. Chem. Biol. 2008 15:950-959), which was subsequently employed as a general approach to derivatize diverse alcohols with desosamine (FIG. 6). The standard assay contains 5 μM PikC_{D50N}—RhFRED, 0.5 mM substrate, 2.5 mM NADPH, 0.25 Unit of glucose-6-phosphate dehydrogenase, and 5 mM glucose-6-phosphate for NADPH regeneration in 100 μl of reaction buffer (50 mM NaH₂PO₄, pH 7.3, 1 mM EDTA, 0.2 mM dithioerythritol, and 10% glycerol). The reaction was carried out at 30° C. for 3 h and terminated by extraction using 3×200 μl of CHCl₃. The resulting organic extract was dried by N₂ and redissolved in 120 μl of methanol. The subsequent LC-MS analysis was performed on a ThermoFinnigan LTQ linear ion-trap instrument (Department of Pharmacology, University of Michigan) equipped with electrospray source and Surveyor HPLC system by using an XBridge™ C18 3.5 μm 150 mm reverse-phase HPLC column under following conditions: mobile phase (A=deionized water+0.1% formic acid, B=acetonitrile+0.1% formic acid), 20% B for 3 min, 20-100% B over 25 min, 100% B for 5 min, 100-20% B over 1 min, 20% B for 15 min; flow rate, 0.21 ml/min The substrate binding assays were performed following the previous report (Li et al., supra).

An enzyme-substrate analysis showed that desosaminyl cyclododecane binds to wild type PikC (PikC_{wt}) with a dissociation constant (K_{d}) of 1,379 μM, about 12 times higher than the K_{d} value (116 μM) of YC-17. The decreased binding affinity of desosaminyl cyclododecane could result from YC-17, an entropic penalty upon binding due to high conformational freedom of the saturated ring system, methymycin) lack of hydrophobic interactions between the functional groups on the macrolactone ring of YC-17 and PikC active site residues, and/or neomethymycin) loss of some specific interactions with desosamine as observed in the co-crystal structure with PikC. When using the more active PikC_{D50N} mutant (Sherman D. H., et al. J. Biol. Chem. 2006 281:26289-26297; Li S., Ouellet H., Sherman D. H. and Podust L. M. J. Biol. Chem. 2009 284:5723-5730), the binding affinities of both carbolide desosaminyl cyclododecane and macrolide YC-17 were shown to be approximately four times higher with K_{d} values of 390 and 32 μM, respectively. Moreover, the self-sufficient fusion enzyme PikC—RhFRED displayed approximately 4-fold enhanced catalytic activity (k_{cat}) compared to PikCwt. Combining these two beneficial properties, the resulting engineered form of the P450 enzyme PikC_{D50N}—RhFRED (k_{cat}/Km=7.44 μM⁻¹·min⁻¹ for YC-17; FIG. 7) is approximately 13 times more active than PikC_{wt} (Li et al., supra). Interestingly, YC-17 and desosaminyl cyclododecane bound to this mutant enzyme with slightly improved K_{d} values of 19 and 309 μM (Table 1), respectively. Due to enhanced substrate conversion and ease of use in the absence of expensive exogenous redox partners, PikC_{D50N}—RhFRED was employed to hydroxylate carbolide desosaminyl cyclododecane and all other substrates.

TABLE 1
The activity of PikC_{D50N}-RhFRED toward various substrates

| | Kₐ | | Number of |
|---|---|---|---|
| Substrates^{a} | (μM) | Yield^{b} | products^{c} |
| 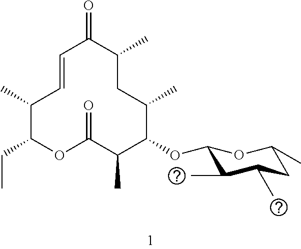 | 19 | >99% | 2 |
| 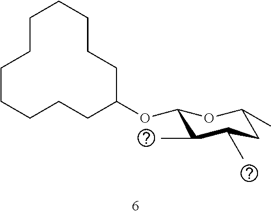 | 309 | 47% | 7 |
| 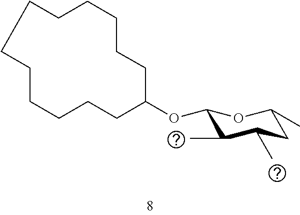 | 218 | 65% | 6 |
| 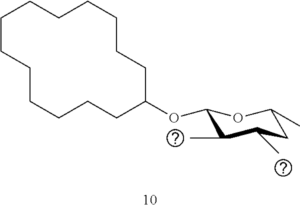 | 289 | 63% | 6 |
| 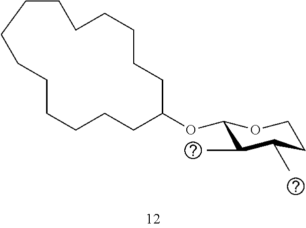 | 243 | 35% | 9 |
| 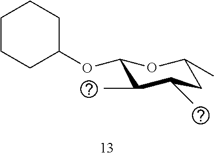 | NB^{d} | 0 | 0 |
| 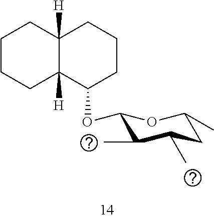 | NB | 0 | 0 |
| 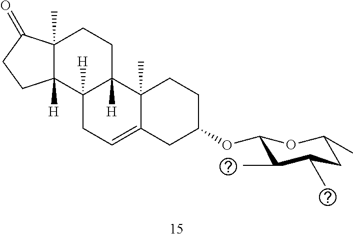 | NB | <1% | 0 |
| 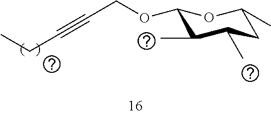 | NB | 0 | 0 |
| 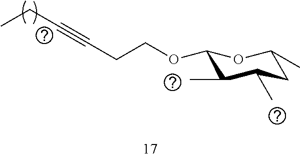 | 2,900 | 8% | 5 |
| 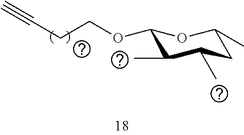 | 2,300 | 14% | 7 |
| 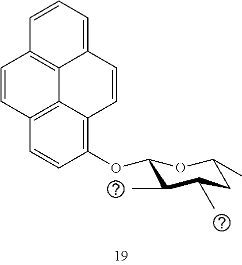 | >5,000 | 4% | 1 |

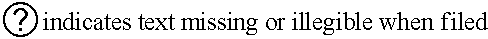

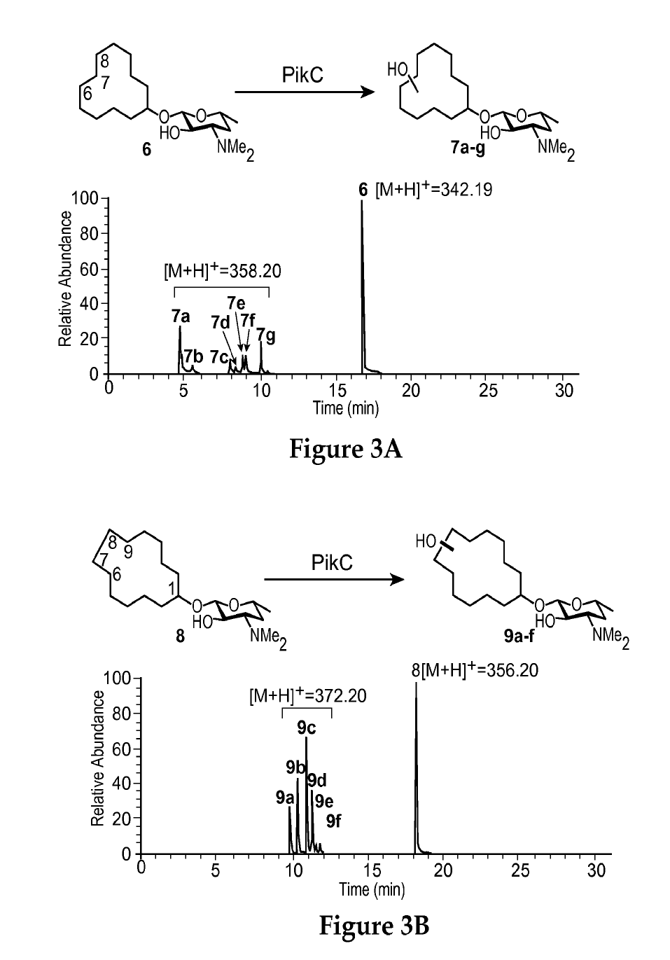

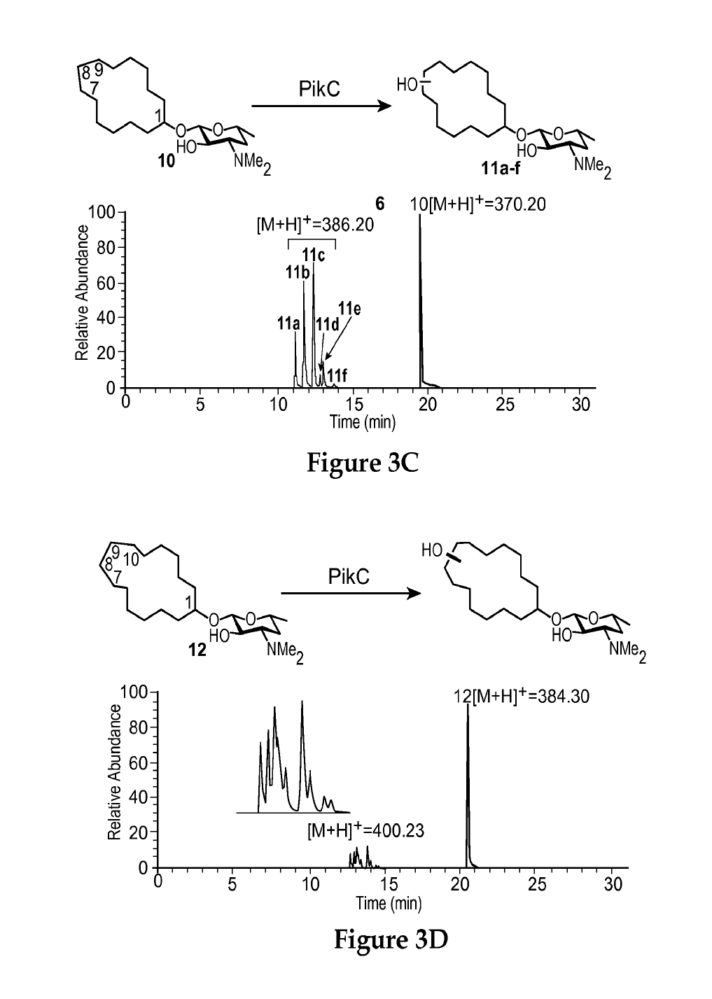

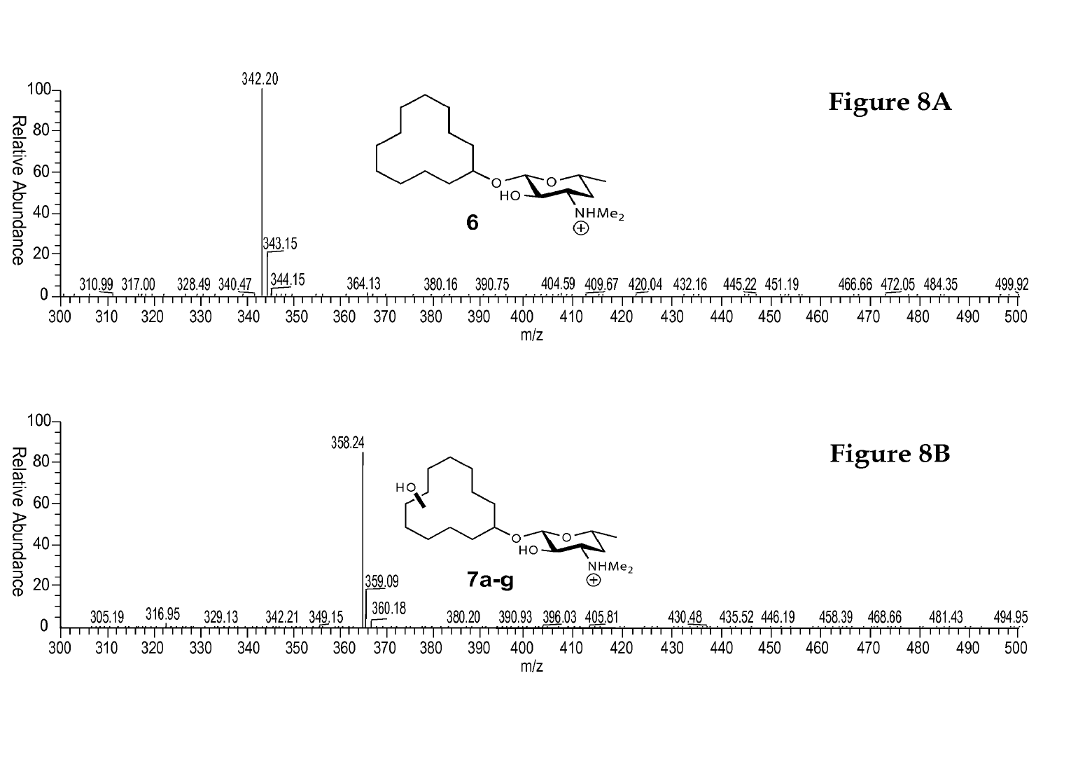

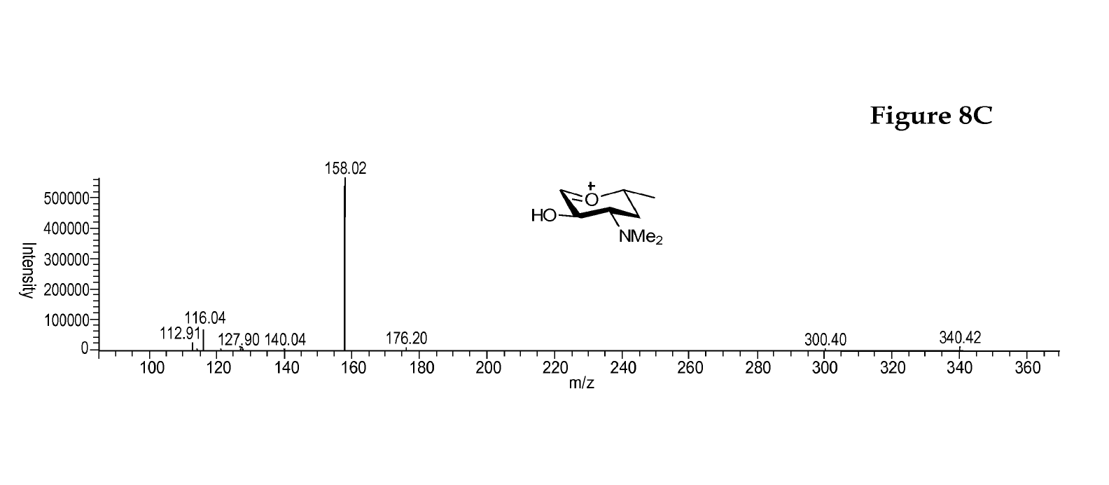

LC-MS analysis (FIG. 3A) of the extract obtained following reaction with PikC_{D50N}—RhFRED showed that 47% of carbolide desosaminyl cyclododecane was converted into seven different monohydroxylated products 7a-g (FIG. 3A; no multi-hydroxylated products were observed) with expected m/z=358.19 for [desosaminyl cyclododecane+OH+H]+using 5 μM of PikC_{D50N}—RhFRED in 3 h (the conversion be driven further by increasing enzyme concentration and/or reaction time). All product ions displayed the same MS/MS spectra (FIG. 8) at m/z=158.02, corresponding to [desosamine-OH]+. The unmodified desosamine moiety indicates that all hydroxylations occur on the cyclododecane ring. In contrast, cyclododecanol lacking an appended desosamine was unable to serve as a substrate for PikC P450 under identical conditions. Therefore, desosamine is indispensable for this biochemical transformation. Notably, PikC_{wt}, PikC_{D50N}, and PikC_{wt}—RhFRED generated similar product profiles compared to PikC_{D50N}—RhFRED, albeit with lower efficiency. These results indicate that neither the point mutation nor the C-terminal RhFRED-fusion with PikC has a significant impact on the binding mode of desosaminyl cyclododecane.

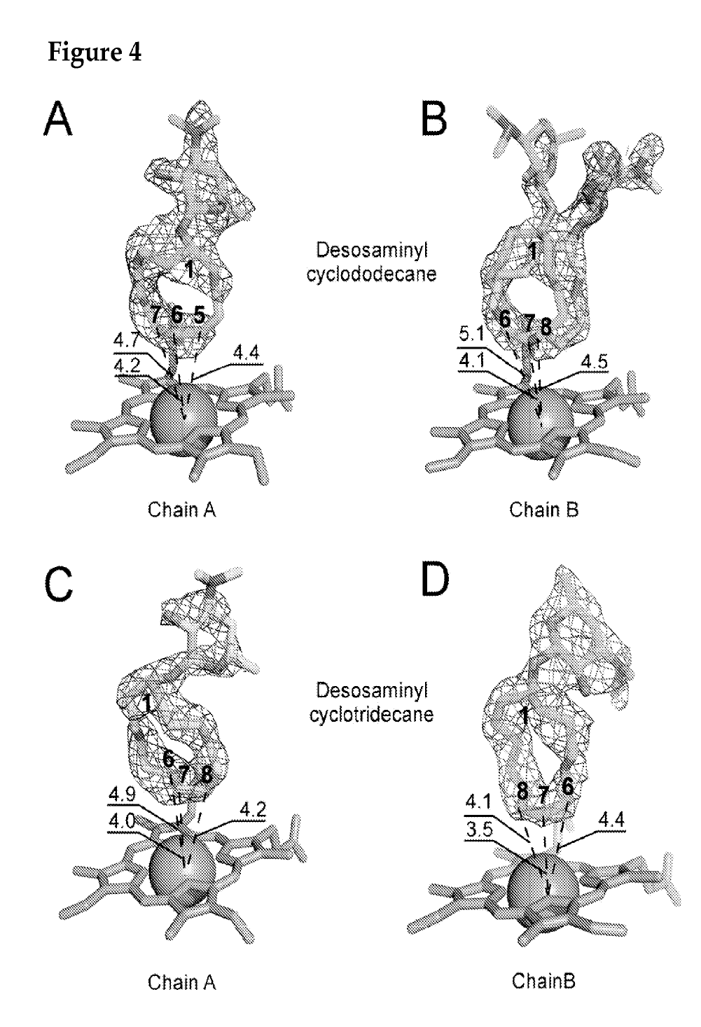

To assess the regio- and stereoselectivity of PikC_{D50N}—RhFRED toward the selected substrate, detailed structural information was obtained on the reaction products. Challenging preparative-scale separations as well as the high similarity of the methylene groups on the cyclododecane ring complicated structure determination of the individual compounds by NMR analysis. Due to the large number of potential isomers (23 total) that could result from oxidation of the 12-membered ring of desosaminyl cyclododecane, it was impractical to synthesize all possible products. Therefore, guided by the 2.0 Å co-crystal structure of mutant PikC_{D50N} enzyme with desosaminyl cyclododecane (FIG. 4), a series of authentic standards with a hydroxyl group installed at the C7 and C6/C8 position (the two diastereotopic carbons were numbered differently for clarity) that were likely to be the major reaction products, were synthesized. During the chemical synthesis, but prior to desosamine installation, racemic mixtures of two diastereomers of a mono-protected diol leading to C6/C8-oxidized authentic materials were prepared. Glycosylation with the glycosyl fluoride of acetate-protected desosamine followed by deprotection provided the four diastereomers that correspond to C6/C8 oxidized products. Similarly, the two diastereomers of C7-oxidized authentic material were obtained.

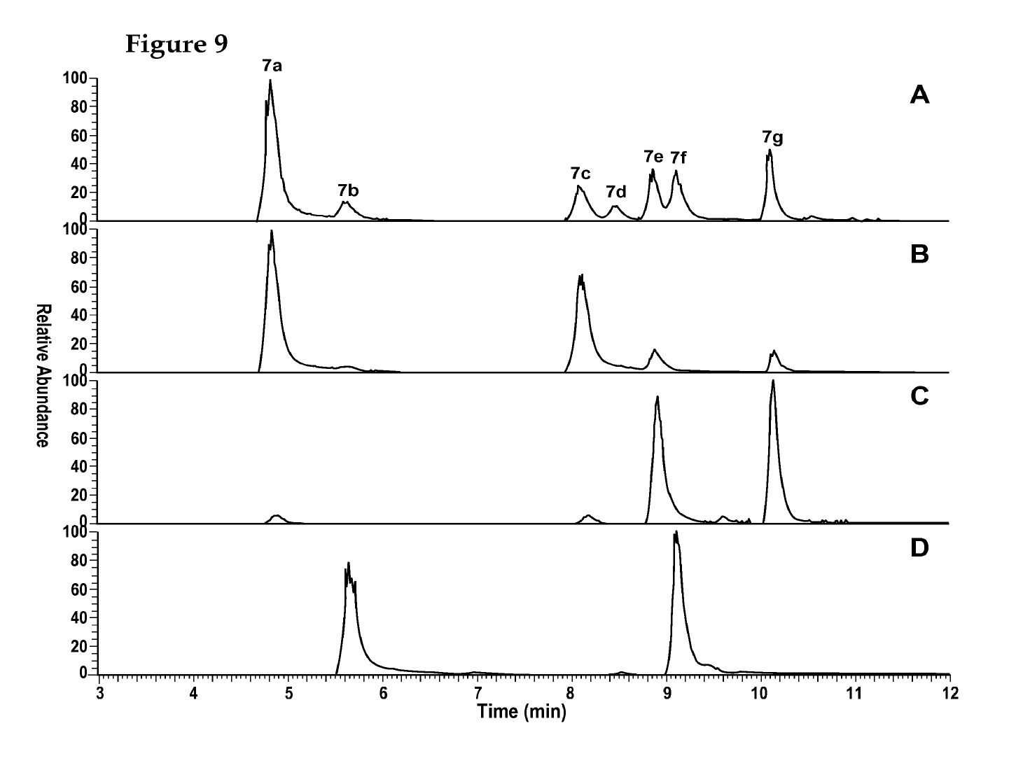

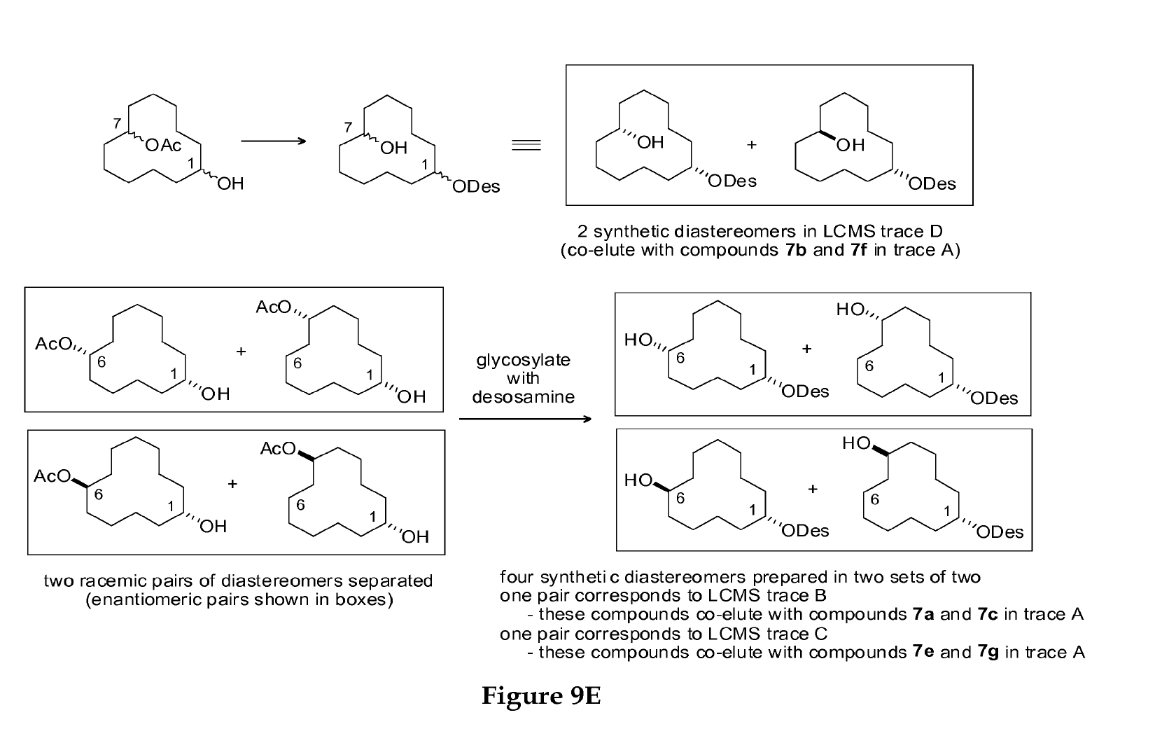

Through comparison of LC-MS retention times and confirmation by co-injections (FIG. 9), six of the seven PikC-derived hydroxylated products of desosaminyl cyclododecane were assigned as the C7 and C6/C8 oxidized materials. Since authentic samples of oxidized products were prepared as diastereomeric pairs, hydroxyl positioning was determined, but the precise stereostructure was not. The ratio of C7 oxidized products (7b and 7f) to C6/C8 oxidized products (FIG. 3A: 7a, 7c, 7e, and 7g) was approximately 1:4. Thus, it is evident that PikC catalyzed hydroxylation occurs primarily at sites most remote from the desosamine-anchoring group, as predicted by the crystal structure (FIG. 4). The C7 and C6/C8 oxidized compounds account for 95% of the mass of monohydroxylated material, and the only unidentified minor product 7d (5%) might be one of the C5 hydroxylated products. Considering the abundance of secondary C—H bonds on the 12-membered ring with almost equal reactivities, this regioselectivity is considerable, but not as strict as that observed toward the native macrolide substrates YC-17 and narbomycin.

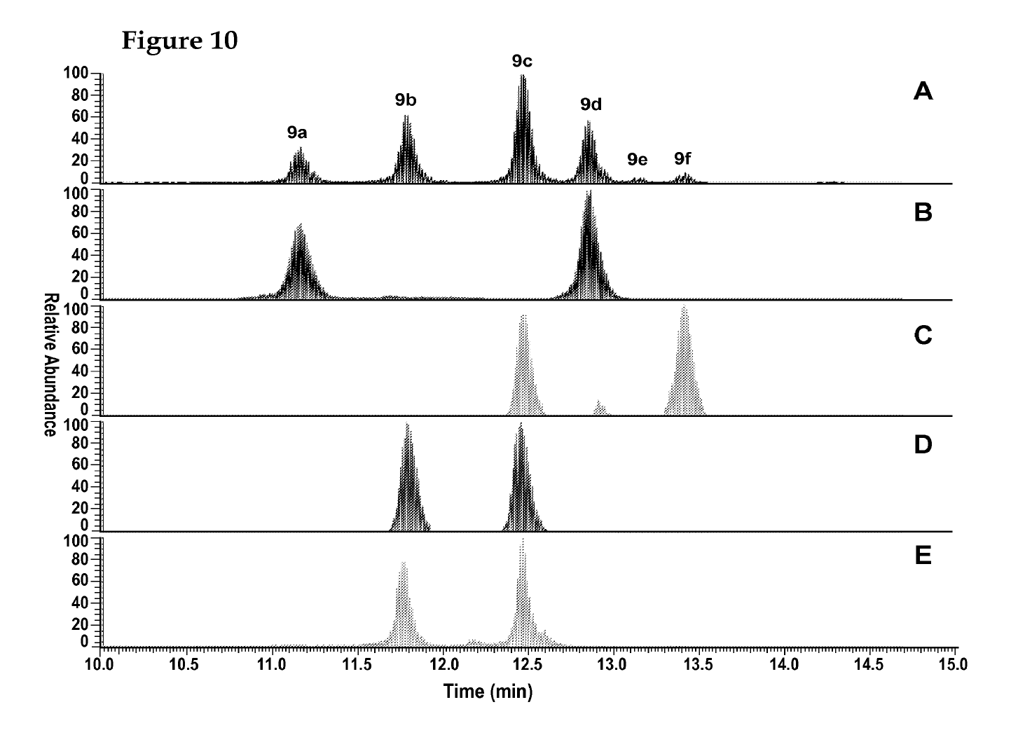

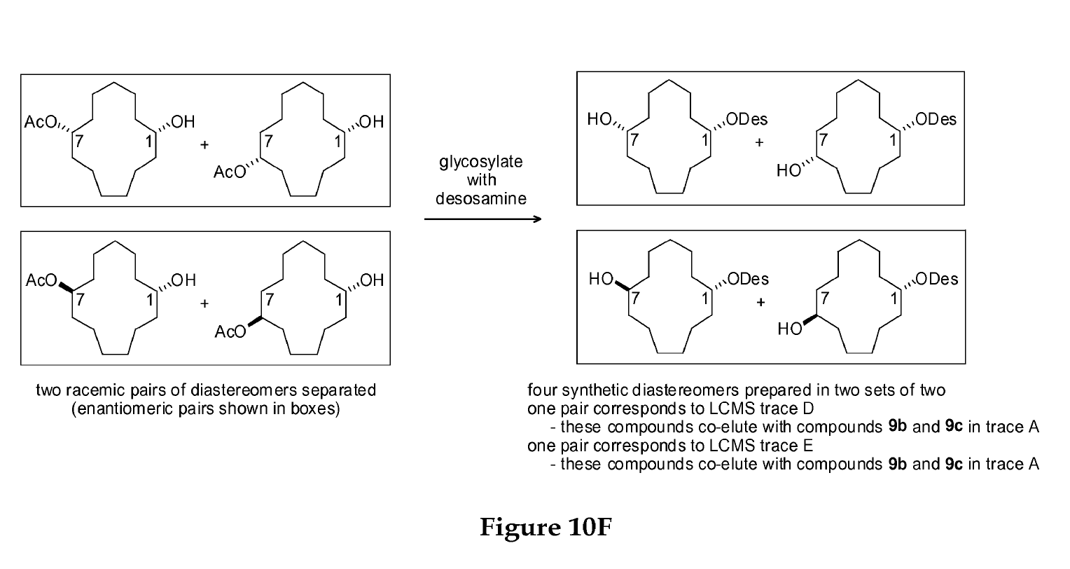

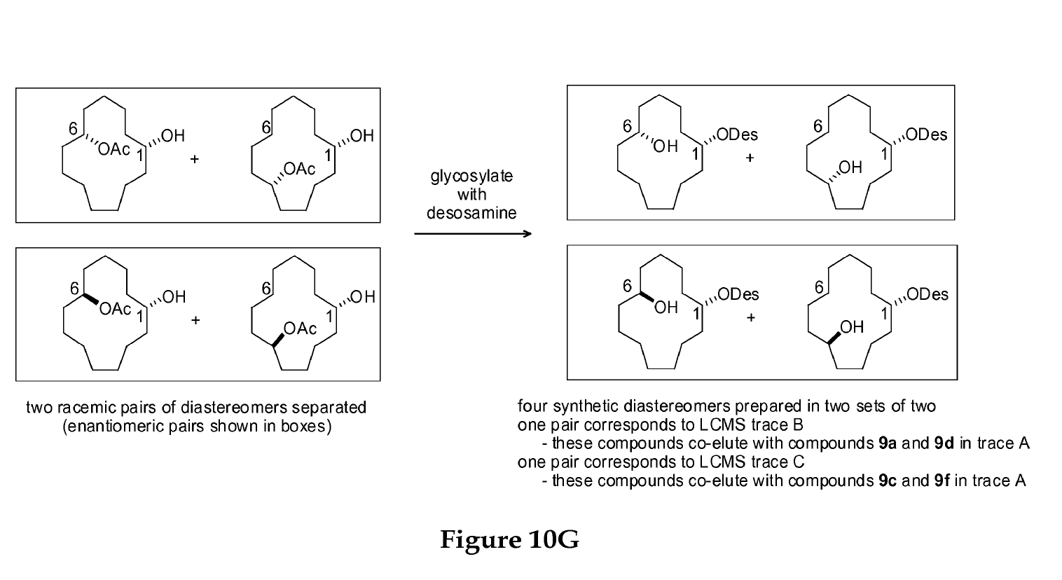

It was investigated whether C—H hydroxylation still occurs at the sites remote from desosamine in the cases of larger hydrocarbon rings, thus the 13-membered ring carbolide 8 (FIG. 3B) was synthesized and treated with PikC_{D50N}—RhFRED in a similar way to desosaminyl cyclododecane. Consistently, the major peaks 9a-d (accounting for 94% of all products, FIG. 3B) were determined to be C7/C8 or C6/C9 mono-hydroxylated products (FIG. 10). However, due to high structural similarity between these molecules, some synthetic hydroxylated authentic standards containing different diastereomeric pairs were not distinguishable from one another due to identical retention times (e.g. FIG. 10, traces D and E), which prevented determination of the exact number of diastereomers generated in the reaction.

Furthermore, two larger cyclic carbolides, including the 14-membered ring 10, and 15-membered ring 12 were investigated. The reactivity of desosaminyl cyclotetradecane 10 was similar to that of 13-membered ring carbolide 8, with 65% substrate converted to hydroxylation products 11a-f (FIG. 3C). In contrast, the desosaminyl cyclopentadecane 12, although having comparable binding affinity with 8 and 10 (Table 1), showed lower conversion (35%) and decreased selectivity reflected by the increased number of products (FIG. 3D), suggesting that this large cyclic substrate might not be located in a suitable position within the PikC active site.

### Example 2

### Analysis of PikC_{D50N}—RhFRED Reactivity Toward Other Types of Desosaminyl Derivatives

Due to the surprising activity and regioselectivity that PikC_{D50N}—RhFRED showed toward 12-, 13-, and 14-membered ring carbolide derivatives, the ability of desosamine to function as an anchor for other classes of compounds was investigated and the activity of PikC_{D50N}—RhFRED toward additional cyclic and linear synthetic substrates (Table 1) was assessed. The results revealed that PikC only marginally hydroxylated cyclic derivatives 13-15, suggesting that these small ring systems might not be accommodated within the active site, or cannot reach the iron-oxo center for catalysis.

To prepare the oxidized product of desosaminyl pyrene 19, an enzymatic reaction containing 0.8 μM PikC_{D50N}—RhFRED, 750 μM glucose-6-phosphate, 0.15 Unit/ml glucose-6-phosphate dehydrogenase, 500 μM NADPH, and 15 mg 19 in 200 ml reaction buffer (50 mM NaH₂PO₄, pH7.3, 1 mM EDTA, 0.2 mM dithioerythritol, and 10% glycerol) was carried out at 30 oC for 20 h. The hydroxylated product 20 (yield ˜8% based on HPLC analysis) was purified by reverse phase C18 preparative HPLC. Upon chloroform extraction from the aqueous solution of 20, 0.2 mg of 20 was recovered. The oxidation site was determined based on the 600M ¹H NMR and COSY performed by using this small amount of 20 in CDCl₃.

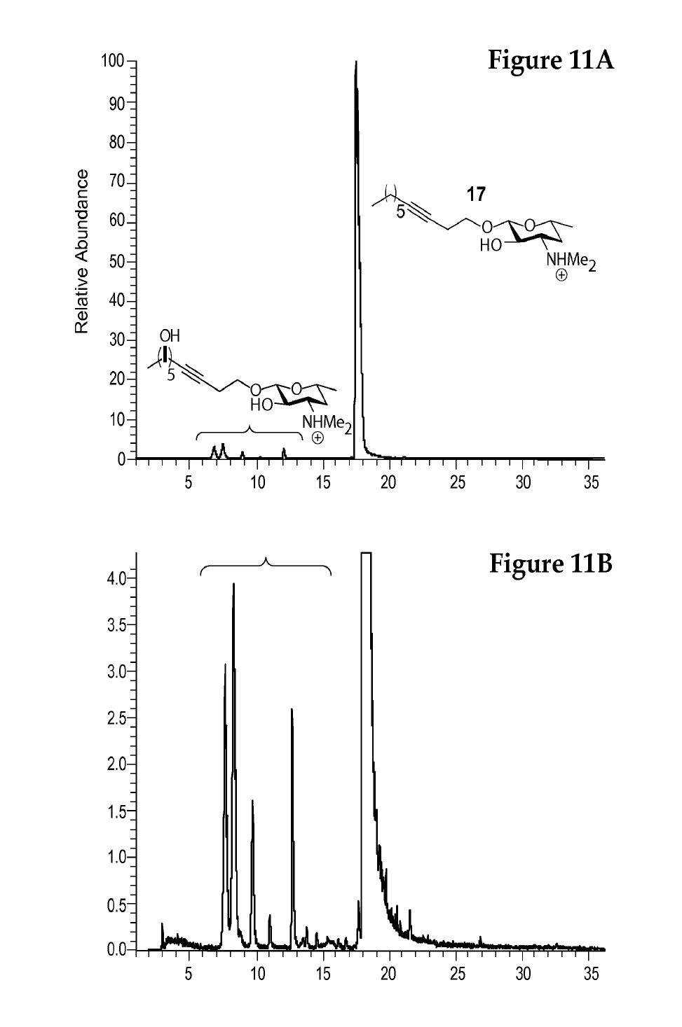

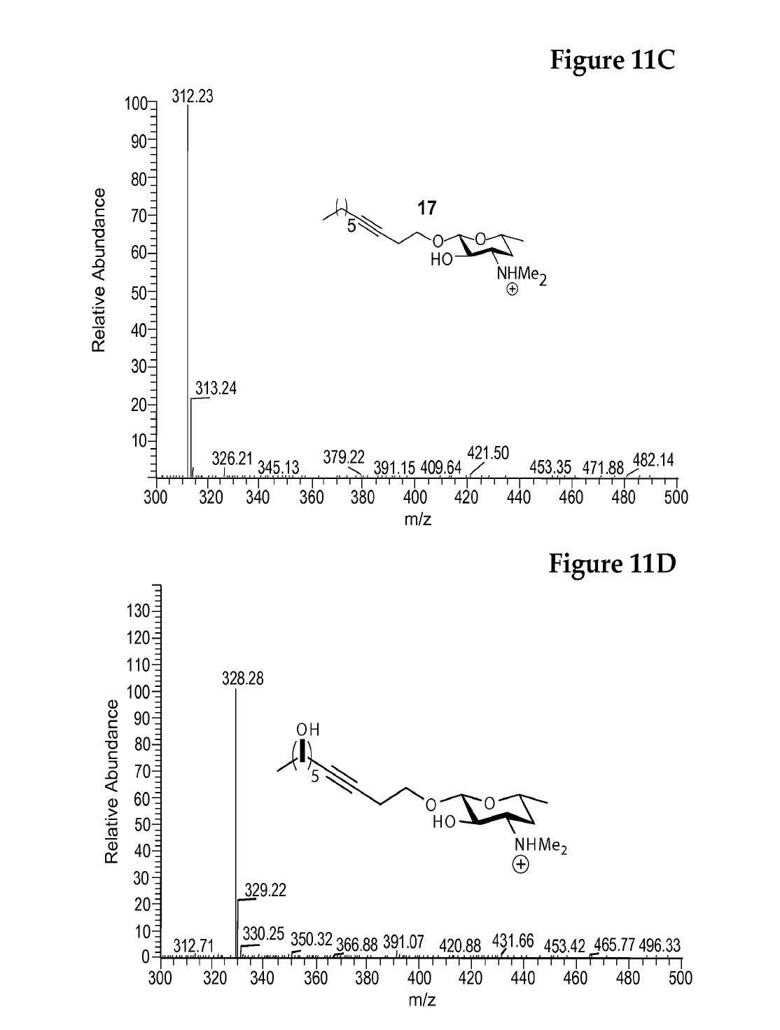

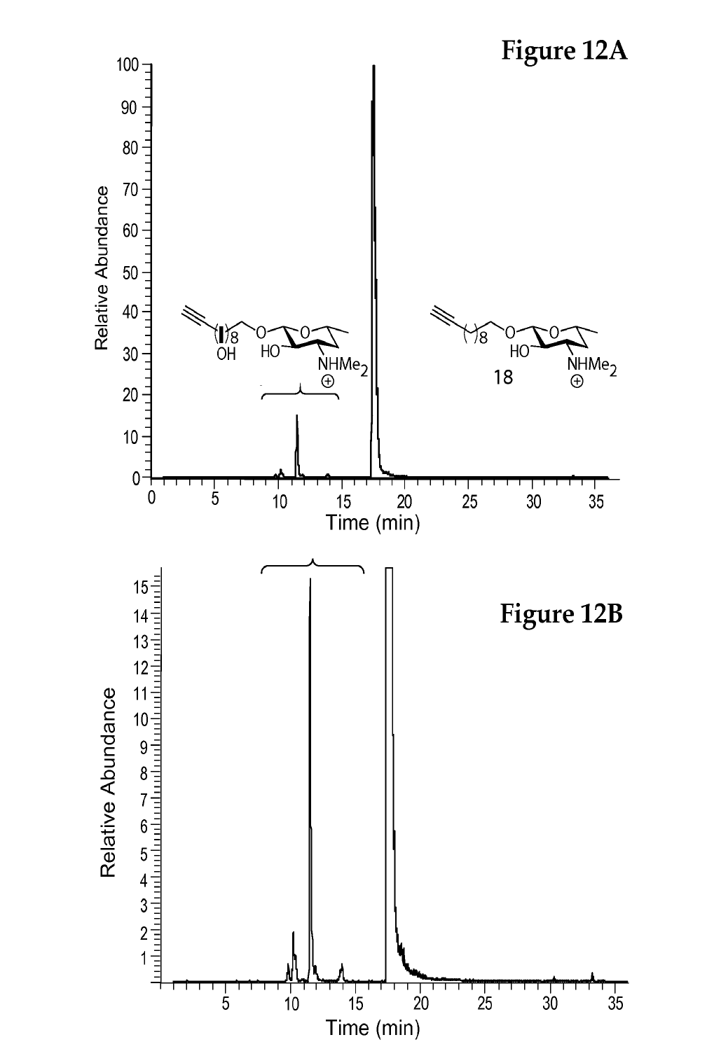

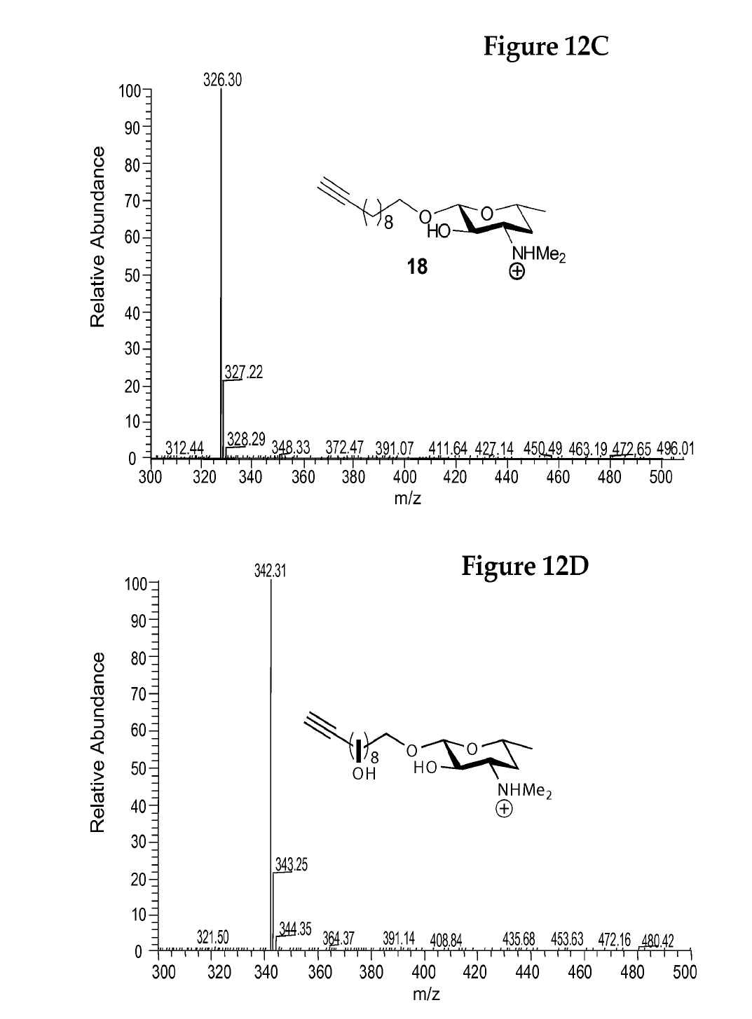

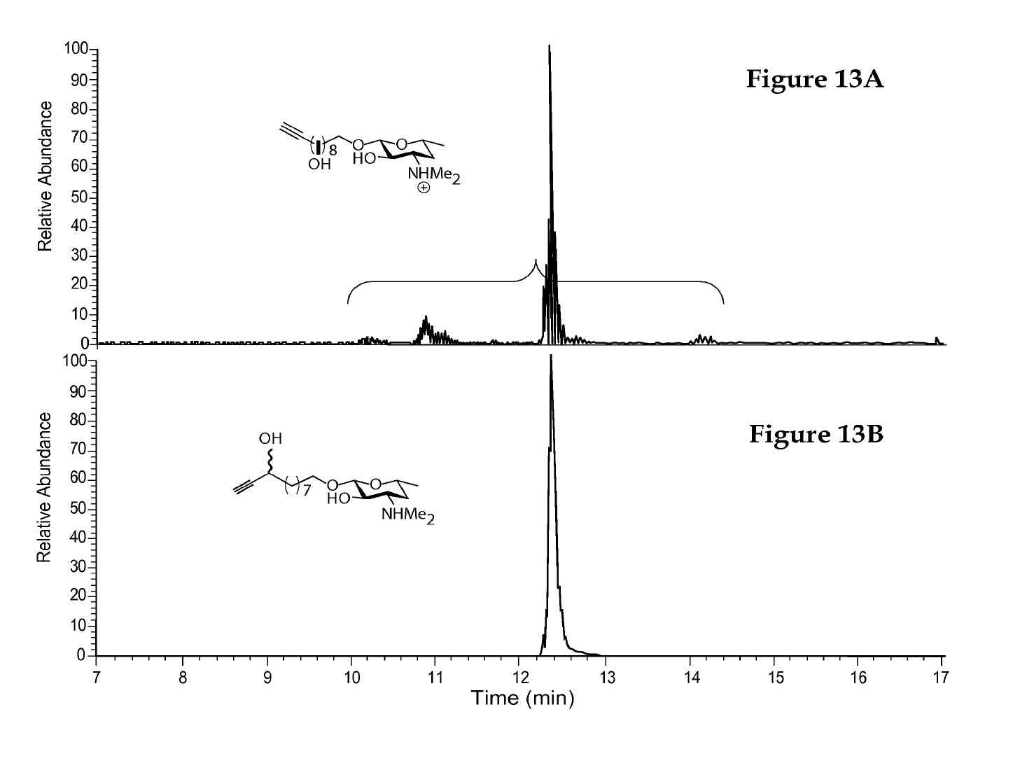

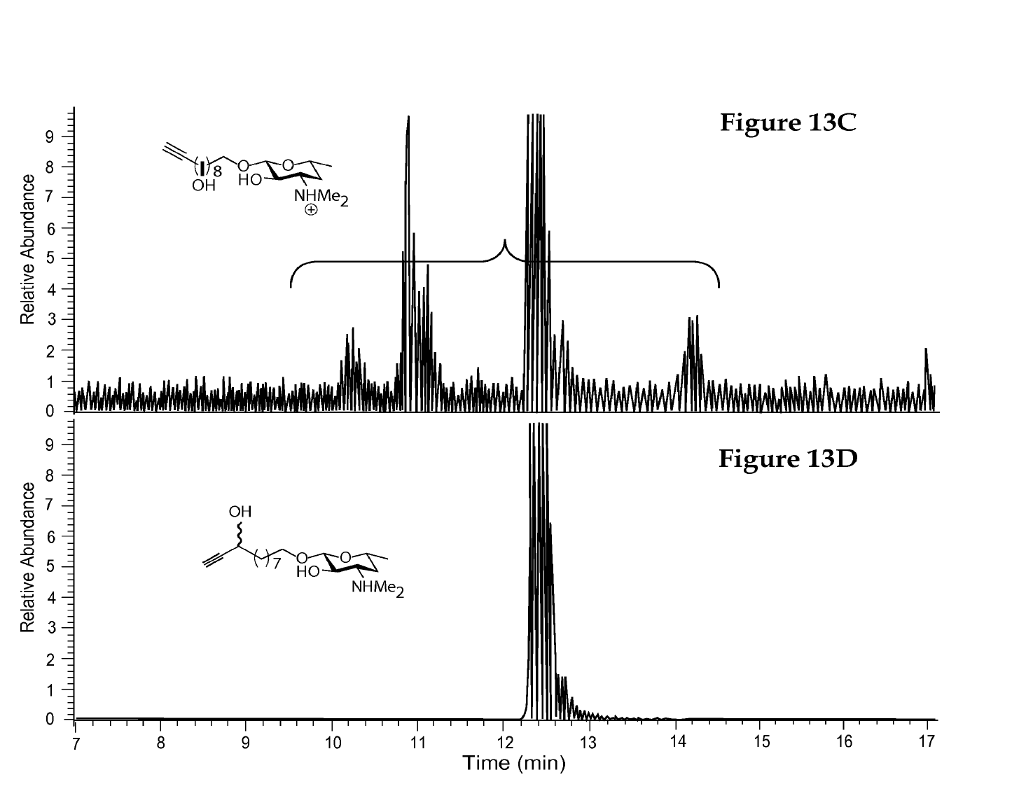

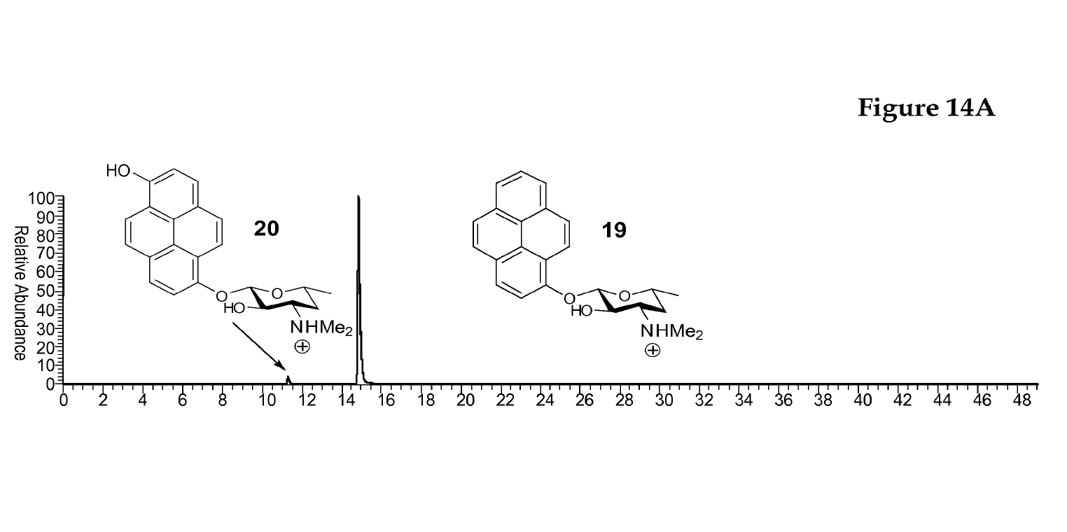

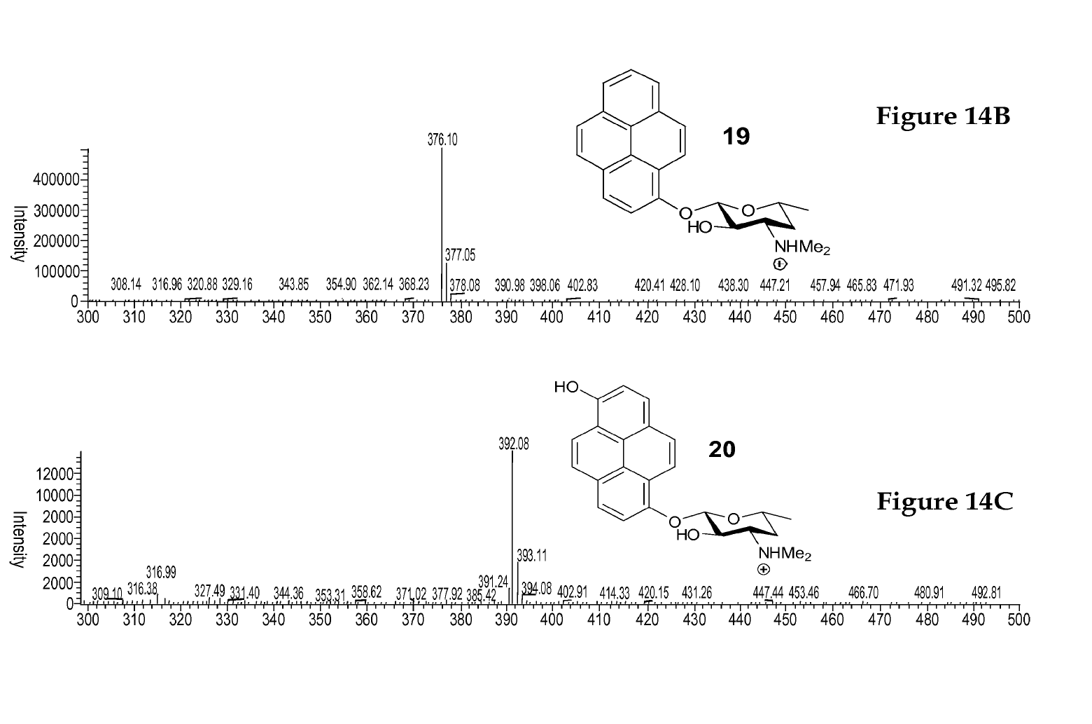

Surprisingly, the linear derivatives 17 and 18 were oxidized to form multiple mono-hydroxylated products with 8% and 14% yields, respectively (Table 1, FIG. 11 and FIG. 12). The predominant hydroxylation site for 18 was identified as the C9 propargylic position (FIG. 13). When aromatic derivative 19 was used as substrate (FIG. 14), a single oxidized product was observed and determined to be the 8-hydroxy desosaminyl pyrene (Table 2), albeit with a conversion of 4%. This observation suggests that rigidity of the substrate might be a key factor to gain high selectivity when using PikC as a biocatalyst for oxidation. The low level of conversion might result from its poor binding and/or higher C—H bond dissociation energy in this aromatic ring system. Taken together, these data reveal that PikC-catalyzed oxidation occurs primarily at the site most remote from the desosamine anchoring group when linear or aromatic substrates are used, suggesting a similar mechanism for control of regioselectivity compared to the carbolides.

TABLE 2
¹H NMR data of 19 and C8-hydroxylated 19

| | Proton assignment (ppm) in CDCl₃ | |
|---|---|---|
| Proton Position | 19 | Hydroxylated 19 |
| H-2 | 7.81 (d, J = 8.3 Hz, 1H) | 7.78 (d, J = 8.3 Hz, 1H) |
| H-3 | 8.12 (d, J = 8.3 Hz, 1H) | 8.02 (d, J = 8.3 Hz, 1H) |
| H-5 | 7.94 (d, J = 9.1 Hz, 1H) | 7.95 (d, J = 9.3 Hz, 1H) |
| H-6 | 8.00 (d, J = 9.1 Hz, 1H) | 8.17 (d, J = 9.1 Hz, 1H) |
| H-8 | 8.13 (d, J = 7.7 Hz, 1H) | — |
| H-9 | 7.96 (t, J = 7.7, 7.7 Hz, 1H) | 7.44 (d, J = 8.1 Hz, 1H) |
| H-10 | 8.14 (d, J = 7.7 Hz, 1H) | 7.96 (d, J = 8.2 Hz, 1H) |
| H-12 | 8.06 (d, J = 9.1 Hz, 1H) | 7.92 (d, J = 9.2 Hz, 1H) |
| H-13 | 8.63 (d, J = 9.1 Hz, 1H) | 8.41 (d, J = 9.1 Hz, 1H) |

The assignment of the aromatic protons of desosaminyl pyrene 19 was conducted based on a full set of 1D and 2D NMR spectra and consistency with the published ¹H NMR data for 1-hydroxypyrene (Applied and Environmental Microbiology, 1994, 60, 3597-3601). The structure of C8-hydroxolated 19 was determined based on the 600M ¹H NMR and COSY spectra, using 19 as reference.

### Example 3

### Analysis of PikC_{D50N}—RhFRED Reactivity Toward Other Types of Derivatives with Alternative Anchoring Groups Other than Desosamine

Desosamine has been demonstrated to be an anchoring group that is capable of directing diverse molecules, including but not limited to a polyketide, a macrolide, a cycloalkane, an aromatic compound, a heteroaromatic compound, or a steroid, into the active site of PikC P450 to achieve regioselective oxidation. Based on recent analysis of x-ray co-crystal structures of PikC (Sherman D. H., et al. The structural basis for substrate anchoring, active site selectivity, and product formation by P450 PikC from Streptomyces venezuelae. J. Biol. Chem. 2006 281:26289-26297; Li S., Ouellet H., Sherman D. H. and Podust L. M. Analysis of transient and catalytic desosamine-binding pockets in cytochrome P-450 PikC from Streptomyces venezuelae. J. Biol. Chem. 2009 284:5723-5730), the positive charged dimethylamino group of desosamine plays a key role in productive substrate binding. Thus, it is hypothesized that alternative groups bearing the dimethylamino group (e.g. 1,2-diol-3-dimethylaminocyclohexane, 1,2-diol-3-methylaminocyclohexane, or N,N-dimethylaminopropylether group) might play a similar role to desosamine as a substrate anchor.

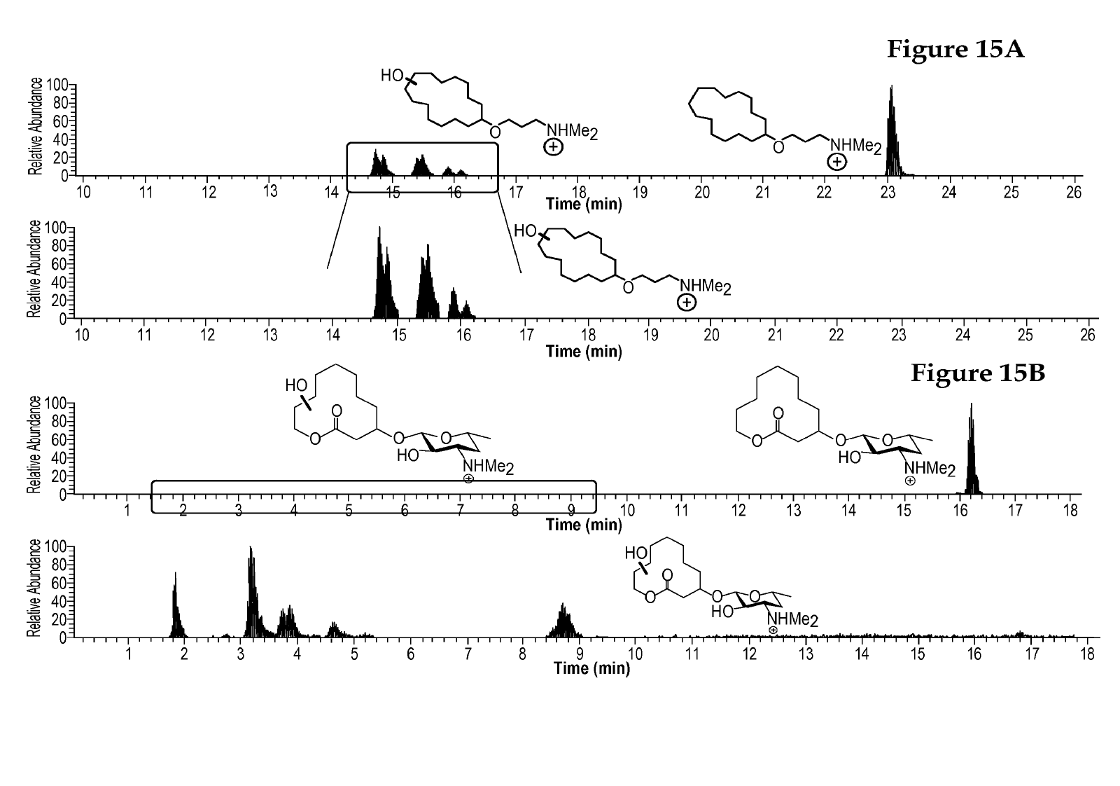

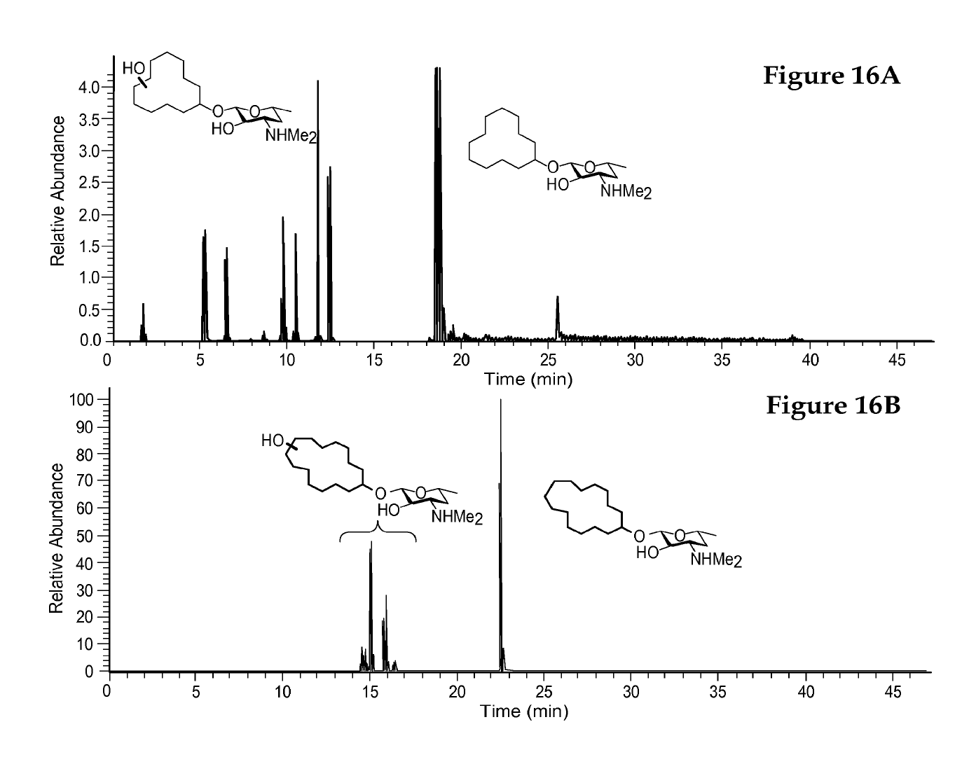

To test this hypothesis, compounds 21-24 were synthesized and the in vitro activity of PikC_{D50N}—RhFRED against these compounds with alternative anchoring groups were evaluated. As shown in FIGS. 15 and 16, a number of mono-hydroxylated products were formed, indicating the functionality of the 1,2-diol-3-dimethylaminocyclohexane, 1,2-diol-3-methylaminocyclohexane, and N,N-dimethylaminopropylether group as substrate anchor to achieve considerable regioselective hydroxylation of cyclic hydrobarbons. Notably, when using the N,N-dimethylaminopropylether group to anchor cyclopentadecanol (24), both yield and selectivity (reflected by the number of product peaks, FIG. 15) were improved compared to 12 (FIG. 3). Since the N,N-dimethylaminopropylether moiety is significantly more chemically friendly than desosaminyl moiety, it represents a more promising anchoring group for industrial application of the current invention.

### Example 4

Structural Basis for Regioselectivity of PikC Toward 12- and 13-Membered Ring Carbolides.

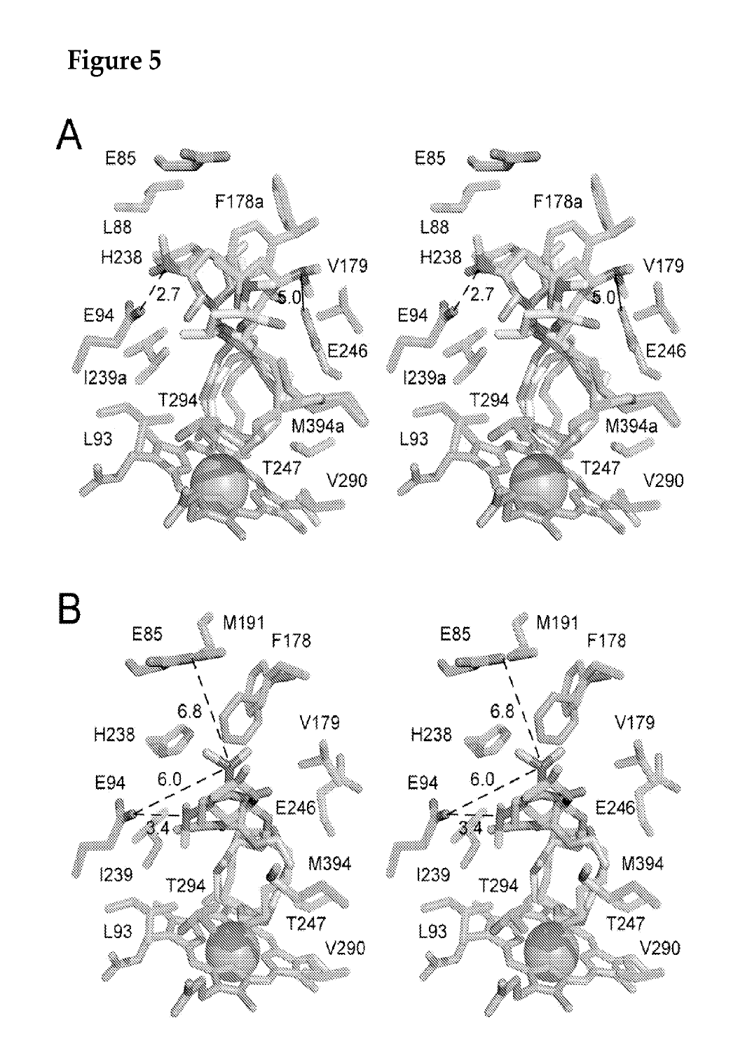

To understand the structural basis for the regioselectivity of PikC and predict the hydroxylation sites of the 12- and 13-membered ring carbolides to direct the synthesis of authentic standards, the crystal structures (Table 3, FIGS. 4 and 5) of PikC_{D50N} in complex with unnatural substrates desosaminyl cyclododecane and 8 (FIGS. 3A and 3B) were solved.

TABLE 3
Crystallographic data and statistics

| | PikC- | | PikC- | |
|---|---|---|---|---|
| | Desosaminyl- | | Desosaminyl- | |
| Protein complex | cyclododecane | | cyclotridecane | |
| PDB ID | PDB ID 2WI9 | | PDB ID 2WHW | |
| Data collection | | | | |
| Wavelength, Å | 1.11587 | | 1.11587 | |
| Resolution, Å | 2.0 | | 2.2 | |
| Unique reflections | 68456 | | 52386 | |
| Redundancy | 3.9 | (3.4)^{a} | 4.2 | (4.3) |
| Completeness, % | 98.2 | (87.7) | 100.0 | (99.8) |
| Space group | P2₁2₁2₁ | | P2₁2₁2₁ | |
| Cell dimensions | 65.3, 109.1, 153.2 | | 60.2, 109.5, 153.1 | |
| (a, b, c), Å ( [symbol] ), ° | 90.0, 90.0, 90.0 | | 90.0, 90.0, 90.0 | |
| Molecules in | 2 | | 2 | |
| asymmetric unit | | | | |
| Solvent content, % | 55.1 | | 55.2 | |
| R_{sym}^{b}, % | 7.2 | (39.0) | 9.8 | (49.7) |
| I/□ | 21.2 | (2.7) | 20.9 | (3.6) |
| Refinement | | | | |
| Reflections used | | | 49541 | |
| in refinement | | | | |
| R_{cryst}(R_{free})^{c}, % | 18.9/23.9 | | 17.6/25.6 | |
| No. of atoms | 6978 | | 7098 | |
| Protein | 6229 | | | |
| Heme | 86 | | 86 | |
| Ligand | 72 | | | |
| Water/ions | 576/15 | | /10 | |
| Wilson plot B-values, Å² | 30.0 | | 29.8 | |
| Mean B-factor, Å² | 31.2 | | 29.46 | |
| Protein | 30.8 | | 27.0 | |
| Heme | 16.4 | | 12.6 | |
| Ligand | A: 62.9, B: 35.3 | | A: 65.9, B: 57.0 | |
| Water/ions | 34.8/85.3 | | 35.3/84.8 | |
| r.m.s. deviations | | | | |

| | | |
|---|---|---|
| Bond length, Å | 0.022 | 0.024 |
| Bond angles, ° | 1.9 | 1.9 |
| Ramachandran^{d} (%) | A: 89.9/9.2/0.6/0.3 | A: 92.0/7.4/0.3/0.3 |
| | B: 92.3/7.7/0.0/0.0 | B: 92.3/7.7/0.0/0.0 |

^{a}Numbers in parentheses correspond to the highest resolution shell.
^{b}R_{sym} = Σ|Iᵢ − <I>|/ΣIᵢ, where Iᵢ is the intensity of the i^{th} observation, and <I> is the mean intensity of reflection.
^{c}R_{cryst} = Σ ∥Fo| − |Fc∥/Σ|Fo|, calculated with the working reflection set. R_{free} is the same as R_{cryst} but calculated with the reserved reflection set.
^{d}Program PROCHECK (Laskowski, R. A., MacArthur, M. W., Moss, D. S. and Thornton J. M. 1993, PROCHECK: a program to check the stereochemical quality of protein structures. J. Appl. Crystallogr. 26, 283-291), portions of the protein residues in most favored/additional allowed/generously allowed/disallowed regions

Crystallization conditions for PikC_{D50N} complexes with desosaminyl cyclododecane and 8 (FIG. 3A-3B) were identified by using commercial high throughput screening kits available in deep-well format (Hampton Research), a nanoliter drop-setting Mosquito robot (TTP Lab Tech) operating with 96-well plates, and a hanging drop crystallization protocol. Optimization of conditions was carried out manually in 24-well plates. The protein from the 1 mM stock was diluted to 0.2 mM by mixing with desosaminyl cyclododecane or 8 dissolved at 2 mM in 10 mM Tris-HCl, pH 7.5. Crystals of PikC_{D50N}-desosaminyl cyclododecane complex were obtained from 15% PEG 4000, 0.1 M Tris-HCl, pH 7.5; 200 mM MgC12. Crystals of the PikC_{D50N}-8 complex were obtained from 12% PEG 8000, 0.1 M sodium cacodilate, pH 6.5, and 200 mM Li₂SO₄. Prior to data collection the crystals were cryo-protected by plunging them into a drop of reservoir solution supplemented with 20% glycerol. Diffraction data were collected at 100-110 K at beamline 8.3.1, Advanced Light Source, Lawrence Berkeley National laboratory, USA. Data indexing, integration, and scaling were conducted using HKL2000 software suite. Crystal structures were determined by molecular replacement using the atomic coordinates of the 2C6H(PDB ID) as a search model (Table 3).

In the co-crystal structure of PikC_{D50N} and desosaminyl cyclododecane, the electron density for desosaminyl cyclododecane is well defined in one monomer of the asymmetric unit (FIG. 4A). In contrast, the other asymmetric unit showed an unambiguously positioned cyclododecane ring, while the dispersed electron density for desosamine indicated at least two alternative conformations (FIG. 4B). A satisfactory fit was achieved when desosaminyl cyclododecane was docked in two flipped orientations. Carbolide desosaminyl cyclododecane binds in the active site in the L-shaped conformation bringing four cyclododecane carbons most remote from the desosamine anchoring group within 5 Å of the Fe reaction center (FIGS. 4A and B), revealing that C5, C6, C7, and C8 are the likely hydroxylation sites. The observed pattern of regioselectivity could arise from sporadic contacts of the desosamine moiety of desosaminyl cyclododecane with a number of amino acid residues (FIG. 5A). Indeed, the specific salt-bridge involving E94 is found in only one conformer (pink in FIG. 5A) of desosaminyl cyclododecane, and a number of the chain A active site residues (green), including F 178, 1239, V242, and M394, adopt alternative conformations indicative of dynamic interactions with the substrate. In chain B, although the salt-bridge to E94 is lost, E246 (ice blue in FIG. 5A) is located within electrostatic distance from the dimethyamino group of the proximal conformer (cyan). However, the E246 side chain is missing from the electron density map of chain A. In addition, the sub-optimal regioselectivity could be due to the inherent flexibility of the large cycloalkane ring. Since all six diastereomers arising from the C7 and C6/C8 hydroxylated regioisomers were observed, there is clearly a lack of stereoselectivity from this enzymatic transformation. The co-crystal structure suggests that compromised stereoselectivity is likely due to flipping (or rotating) of the carbolide substrate in the PikC active site resulting in oxidation on both faces of the ring (FIGS. 4A and B).

Further inspection of the co-crystal structures revealed that whereas three orientations are distinguishable for 6, only two orientations (one in each protein monomer in the asymmetric unit) are observed for 8 in the 2.2 Å x-ray co-crystal structure (FIGS. 4 and 5), suggesting improved complementarity and/or more limited conformational freedom of the larger ring in the active site. This could explain the increased binding affinity (K_{d}=218 μM) and hence reactivity (65% yield) of 8 compared to desosaminyl cyclododecane (K_{d}=309 μM, 47% yield). In one monomer (cyan in FIG. 5B), the desosamine moiety is in salt-bridge contact with E94, while in the other monomer (pink), it is within electrostatic distance from both E94 and E85. Amino acid side chains in the active site are stabilized in a single conformation, with the exception of F178, which is represented by different conformers in chain A (green) and chain B (ice blue) (FIG. 5B). Similar to desosaminyl cyclododecane, the co-crystal structure suggests that flipping-over (or rotation) of 8 in the active site could enable hydroxylation (likely at C6/C9 or C7/C8) on both faces of the ring. However, a more limited resolution of the product profile in the LCMS analysis (FIG. 10) (unlike oxidations of desosaminyl cyclododecane, where oxidation products were better distinguished by LCMS, FIG. 9) prevents verification of this prediction, or further evaluation of the reaction stereoselectivity.

Despite previously demonstrated dynamics of PikC (Sherman D. H., et al. The structural basis for substrate anchoring, active site selectivity, and product formation by P450 PikC from Streptomyces venezuelae. J. Biol. Chem. 2006 281:26289-26297), no fit-induced conformational changes were observed in response to binding of the various carbolides, which could potentially prevent the substrate from wobbling in the active site. This observation contradicts the concept of “conformational plasticity” applied for mammalian P450 enzymes (Muralidhara B. K. and Halpert J. R. Thermodynamics of ligand binding to P450 2B4 and P450eryF studied by isothermal titration calorimetry. Drug. Metab. Rev. 2007 39:539-556), and limits the role of PikC conformational dynamics to substrate access to and product release from the active site.

### Example 5

### Antibacterial Activities of Synthetic Desosaminyl Derivatives

In macrolide antibiotics, desosamine (or a related deoxysugar moiety) has been found to play a crucial role in the interaction of these important compounds with the 23S ribosomal RNA (the major drug target for macrolides) through a number of specific contacts with ribonucleotides in the peptidyl-transferase center (Schliinzen F., et al. Structural basis for the interaction of antibiotics with the peptidyl transferase centre in eubacteria. Nature 2001 413:814-821). Accordingly, these synthetic desosaminyl derivatives possess antibacterial activity.

Two-fold serial dilutions of 40 mM (10 mM for positive control erythromycin) DMSO solutions of test compounds with DMSO generated a series of stock solutions with concentrations ranging from 0.31˜40 mM. Then, a ten-time dilution of each was performed by using dd H₂O to make a 31˜4000 μM series. Cultures of target strains were grown in appropriate media at 37° C. (30° C. for Deinococcus radiodurans) with shaking (180 rpm). An overnight seed culture was diluted to an OD600 of 0.05, grown to an OD600 of 0.4˜0.6, back diluted to an OD600 of 0.004, and 45 μL, of this diluted culture was added to each well of a 384-well microtiter plate that contained 5 μL, of a given dilution of compound in dd H₂O. Plate cultures were grown for 16 h (60 h for D. radiodurans) and OD₆₀₀ measurements were taken. All measurements were performed in duplicate.

As shown in Table 4, the cyclic carbolides including desosaminyl cyclododecane, 8, 10, and 12 (FIG. 3A-3D) displayed some inhibitory activities against selected microbial targets, while their corresponding aglycones were inactive, confirming the significance of desosamine for bioactivity. Remarkably, the aromatic derivative 19 (Table 1) showed similar or even higher bioactivity compared to the natural macrolide antibiotics methymycin and pikromycin. The bioactivity of 8 was compared with its synthetic hydroxylated products. Unexpectedly, upon installation of a hydroxyl group at either the C7/C8 or C6/C9 positions (the four authentic standards, each of which contains a pair of diastereomers), the products lost activity. Although hydroxylation of 8 had a detrimental impact on biological activity against a limited number of microbial targets, installation of this functional group might enable a useful chemical handle for further functionalization (Rentmeister A., Arnold F. H. and Fasan R. Chemo-enzymatic fluorination of unactivated organic compounds. Nat. Chem. Biol. 2009 5:26-28) and subsequent generation of more potent therapeutics. Further analysis of these carbolides, and direct investigation of their presumed binding to ribosomal targets will provide important insights into the impact of PikC-mediated regioselective hydroxylation for development of new small molecules for treatment of microbial pathogens and other human diseases.

TABLE S3
Antibacterial activities of desosaminyl derivatives against selected strains

| | Minimal inhibitory concentration (μM) | | | | | | | |
|---|---|---|---|---|---|---|---|---|
| | | | | | | | | Multidrug |
| | Kocuria | Staphylococcus | Bacillus | | | | | resistant |
| | rhizophila | aureus | subtillis | Deinococcus | Escherichia. | S. aureus | Acinetobacter | S. aureus |
| | ATCC9341^{a} | ATCC6538P | DHS5333 | radiodurans^{b} | coli TolC^{c} | NorA^{d} | baumannii | (MRSA) |
| Media | Nutrient broth | LB | LB | Special media^{f} | Mueller Hinten | Mueller Hinten | Mueller Hinten | Mueller Hinten |
| Compound | | | | | | | | |
| Erythromycin | <0.8 | <0.8 | <0.8 | 6.2 | <0.8 | <0.8 | 25 | >100 |
| DMSO | — | — | — | — | — | — | — | — |
| 1 | 100 | 100 | 100 | 400 | 100 | 100 | >400 | >400 |
| 2 | 50 | 100 | 100 | 200 | 50 | 100 | >400 | >400 |
| 4 | <3.1 | 6.2 | <3.1 | 100 | 25 | 3.1 | >400 | >400 |
| 5 | 6.2 | 25 | 12.5 | 100 | 100 | 12.5 | >400 | >400 |
| 6 | 50 | >400 | 200 | 100 | 50 | 200 | >400 | 400 |
| 8 | 50 | 400 | 200 | 100 | 50 | 200 | >400 | 200 |
| 10 | 50 | >400 | 200 | 50 | 50 | 400 | >400 | 400 |
| 12 | 50 | 200 | 100 | 50 | 50 | 100 | >400 | 100 |
| 13 | >400 | >400 | >400 | >400 | >400 | >400 | >400 | >400 |
| 14 | >400 | >400 | >400 | >400 | >400 | >400 | >400 | >400 |
| 15 | >400 | >400 | >400 | >400 | >400 | >400 | >400 | >400 |
| 8-C6OH-a^{e} | >400 | >400 | >400 | >400 | >400 | >400 | >400 | >400 |
| 8-C6OH-b^{e} | >400 | >400 | >400 | 400 | >400 | >400 | >400 | >400 |
| 8-C7OH-a^{e} | >400 | >400 | >400 | 400 | >400 | >400 | >400 | >400 |
| 8-C7OH-b^{e} | >400 | >400 | >400 | 100 | 400 | >400 | >400 | >400 |
| 17 | >400 | >400 | >400 | >400 | >400 | >400 | >400 | >400 |
| 18 | >400 | >400 | >400 | >400 | 400 | >400 | >400 | >400 |
| 19 | 25 | 25 | 6.2 | 25 | 25 | 25 | 100 | 25 |
| Cyclododecanol | >400 | >400 | >400 | >400 | >400 | >400 | >400 | >400 |
| Cyclotridecanol | >400 | >400 | >400 | >400 | >400 | >400 | >400 | >400 |
| Cyclopentadecanol | >400 | >400 | >400 | >400 | >400 | >400 | >400 | >400 |

^{a}Previously known as Micrococcus luteus ATCC 9341, which is sensitive to macrolide antibiotics.
^{b}A gift from Prof. Ada E. Yonath (Structural Biology Department, Weizmann Institute of Science, Rehovot, Israel)
^{c}E. coli W3110 TolC disruption mutant that is more sensitive to antibiotics
^{d}S. aureus 8325 NorA disruption mutant that is more sensitive to antibiotics
^{e}8-C6OH-a, b and 8-C7OH-a, b correspond to synthetic C6/C9 and C7/C8 hydroxylated standards of 13-membered carbolide, each of which contains a pair of diastereomers as shown in trace B, C and D, E, respectively in FIG. S6
^{f}Media recipe: 10 g caseine peptone (tryptic digest), 5 g yeast extract, 5 g NaCl and 5 g glucose (add after sterilization) in 1 liter water, pH 7.2.

### Example 6

### General Organic Synthesis Protocols

General Organic Synthesis Protocols. All reagents were used as received unless otherwise noted. Solvents were purified under nitrogen using a solvent purification system (Innovative Technology, inc., Model # SPS-400-3 and PS-400-3. Ni(COD)₂ (Strem Chemicals, Inc., used as received), 1,3-Bis(2,4,6-trimethyl-phenyl)imidazolium chloride (IMes·HCl), 1,3-Bis(2,6-di-iso-propylphenyl)imidazolium chloride (IPPHCl), and potassium tertbutoxide were stored and weighed in an inert atmosphere glovebox. All reactions were conducted in flame-dried glassware under nitrogen atmosphere. ¹H and 13C spectra were obtained in CDCl₃ at RT (25° C.), unless otherwise noted, on a Varian Mercury 400 or Varian Unity 500 MHz instrument. Chemical shifts of ¹H NMR spectra were recorded in parts per million (ppm) on the 6 scale from an internal standard of residual chloroform (7.27 ppm). Chemical shifts of ¹³C NMR spectra were recorded in ppm from the central peak of CDCl₃ (77.0 ppm) on the 6 scale. Low resolution electrospray mass spectra were obtained on a Micromass LCT spectrometer, low resolution chemical ionization mass spectra were obtained on a Micromass VG-70-250-S spectrometer, and high resolution electrospray mass spectra were obtained on a Micromass AutoSpec Ultima spectrometer at the University of Michigan Mass Spectrometry Laboratory.

Isolation of Desosamine Diacetate from (−)-Erythromycin Hydrate as the Bis(Acetate) Protected Desosamine

(3R,4S)-4-(dimethylamino)-6-methyltetrahydro-2H-pyran-2,3-diyl diacetate A modification of a literature procedure was followed (Chen, H.; Yamase, H.; Murakami, K.; Chang, C.; Zhao, L.; Zhao, Z.; Liu, H. Biochemistry. 2002, 41, 9165). To a solution of 7.0 g erythromycin hydrate (7.043 g, 9.54 mmol) (Aldrich) in 50 mL ethanol in a 250 mL round bottomed flask, 130 mL 6N HCl was added slowly at rt. The mixture was heated at reflux for 3 h and was allowed to cool to rt. The reaction mixture was then washed with 50 mL of chloroform three times and the orange colored aqueous layer was concentrated under vacuum to obtain crude 4-(dimethylamino)-6-methyltetrahydro-2H-pyran-2,3-diol as an orange solid, which was subjected to the next step without further purification. To the suspension of crude desosamine in acetic anhydride (10 mL), 2 mL of conc. H₂SO₄ was added at 0° C. and stirred at rt for 12 h. The reaction mixture was poured into ice water and neutralized slowly with solid sodium bicarbonate and extracted with dichloromethane. The combined organic layer was concentrated and purified by column chromatography (Si0₂, 95:5 CH₂Cl₂:MeOH) to afford 4-(dimethylamino)-6-methyltetrahydro-2Hpyran-2,3-diyldiacetate (2.189 g, 8.45 mmol, 88% over 2 steps) as a light orange liquid. (Spectral data of the compound was identical with that previously reported; Davidson, M. H.; McDonald, Org. Lett. 2004, 6, 1601).

Conversion of Bis-Acetate into the Glycosyl Fluoride

(Damager I.; Numao, S.; Chen, H.; Brayer G. D.; Withers S. G. Carbohydrate Research. 2004, 339 1727) (2R,3R,4S,6R)-4-(dimethylamino)-2-fluoro-6-methyltetrahydro-2H-pyran-3-yl acetate To a stirring solution of HF/pyridine (1.50 mL) in a clean and dry polyethylene vial, the solution of 4-(dimethylamino)-6-methyltetrahydro-2H-pyran-2,3-diyl diacetate (240 mg, 0.92 mmol) in toluene (1 mL) was added and stirred for 30 min at 0° C. The reaction mixture was diluted with 10 mL brine solution, neutralized with NaHCO₃ and extracted with ethyl acetate. The combined organic layers were dried with MgSO₄, filtered, concentrated, and purified by column chromatography (SiO₂, ethyl acetate) to afford the α-anomer of 4-(dimethylamino)-2-fluoro-6-methyltetrahydro-2H-pyran-3-yl acetate (160 mg, 0.73 mmol, 79%) as a clear oil.

General Procedure for Glycosylation with Desosamine

(Anzai, Y.; Li, S.; Chaulagain, M. R.; Kinoshita, K.; Kato, F.; Montgomery, J.; Sherman, D. H. Chem. Biol. 2008, 15, 950) A suspension of alcohol (1.0 equiv), 4-(dimethylamino)-2-fluoro-6-methyltetrahydro-2H-pyran-3-yl acetate (2.0 equiv) and molecular sieves (4A) in dichloromethane was stirred for 30 min. BF₃.OEt₂ (4.0 equiv, freshly distilled from CaH₂) was added to the ice cooled reaction mixture and stirred for 1 h at 0° C. The reaction mixture was neutralized with saturated sodium bicarbonate solution and extracted with ethyl acetate. The combined organic layers were dried with MgSO₄, filtered, concentrated, and purified by column chromatography (SiO₂, 1:19 MeOH:CH₂Cl₂).

### General Procedure for Acetate Deprotection

To the solution of the acetate obtained above (1 equiv) in methanol (20 mL/mmol), K₂CO₃ (4 equiv) was added as a solid and stirred at rt until the starting material was consumed as judged by TLC analysis. The reaction mixture was diluted with brine and extracted with ethyl acetate. The combined organic layers were dried with MgSO₄, filtered, and concentrated to afford the deprotected desosamine conjugate.

2-(Cyclododecyloxy)-4-(dimethylamino)-6-methyltetrahydro-2H-pyran-3-yl acetate. Following the general procedure, cyclododecanol (19 mg, 0.11 mmol), 4-(dimethylamino)-2-fluoro-6-methyltetrahydro-2H-pyran-3-yl acetate (22 mg, 0.10 mmol), molecular sieves (100 mg) and BF₃.OEt₂ (20 μL, 0.2 mmol) were employed to obtain 2-(cyclododecyloxy-4-(dimethylamino)-6-methyltetrahydro-2H-pyran-3-yl acetate (20 mg, 0.052 mmol, 52%) after column chromatography (SiO₂, ethyl acetate) as a white solid.

2-(Cyclododecyloxy-4-(dimethylamino)-6-methyltetrahydro-2H-pyran-3-ol (6). Following the general procedure, 2-(cyclododecyloxy-4-(dimethylamino)-6-methyltetrahydro-2H-pyran-3-yl acetate (20 mg, 0.052 mmol), K₂CO₃ (27 mg, 0.2 mmol), and methanol were employed to afford 2-(cyclododecyloxy-4-(dimethylamino)-6-methyltetrahydro-2H-pyran-3-ol (15 mg, 0.045 mmol, 86% yield) as a white solid.

2-(Cyclotridecyloxy)-4-(dimethylamino)-6-methyltetrahydro-2H-pyran-3-yl acetate. Following the general procedure, cyclotridecanol (21 mg, 0.11 mmol, prepared by NaBH₄ reduction of cyclotridecanone), 4-(dimethylamino)-2-fluoro-6-methyltetrahydro-2H-pyran-3-yl acetate (30 mg, 0.14 mmol), molecular sieves (150 mg) and BF₃.OEt₂ (28 μL, 2.8 mmol) were employed to obtain 2-(cyclotridecyloxy)-4-(dimethylamino)-6-methyltetrahydro-2H-pyran-3-yl acetate (28 mg, 0.07 mmol, 64%) after column chromatography (SiO₂, ethyl acetate) as a white solid.

2-(Cyclotridecyloxy)-4-(dimethylamino)-6-methyltetrahydro-2H-pyran-3-ol (8). Following the general procedure, cyclotridecanol (21 mg, 0.11 mmol, prepared by NaBH₄ reduction of cyclotridecanone), 4-(dimethylamino)-2-fluoro-6-methyltetrahydro-2H-pyran-3-yl acetate (30 mg, 0.14 mmol), molecular sieves (150 mg) and BF₃.OEt₂ (28 μL, 2.8 mmol) were employed to obtain 2-(cyclotridecyloxy)-4-(dimethylamino)-6-methyltetrahydro-2H-pyran-3-yl acetate (28 mg, 0.07 mmol, 64%) after column chromatography (SiO₂, ethyl acetate) as a white solid.

Cyclotetradecanol. To the solution of (E)-cyclotetradec-2-enol (9 mg, 0.043 mmol) in 2 mL dry ethyl acetate under nitrogen, PtO₂ (2 mg, 0.007 mmol) was added and purged with hydrogen gas in a balloon with a needle and stirred for 2 h (Oppolzer, W.; Radinov, R. N.; El-Sayed, E. J. Org. Chem. 2001, 66, 4766; Knapp-Reed, B.; Mahandru, G. M.; Montgomery, J. J. Am. Chem. Soc. 2005, 127, 13156). The reaction mixture was filtered through a small silica plug and concentrated to afford cyclotetradecanol (8 mg, 0.038 mmol, 89%) as a white solid.

2-(Cyclotetradecyloxy)-4-(dimethylamino)-6-methyltetrahydro-2H-pyran-3-yl acetate. Following the general procedure, cyclotetradecanol (8 mg, 0.038 mmol), 4-(dimethylamino)-2-fluoro-6-methyltetrahydro-2H-pyran-3-yl acetate (10 mg, 0.045 mmol), molecular sieves (100 mg) and BF₃.OEt₂ (10 μL, 0.1 mmol) were employed to obtain 2-(cyclotetradecyloxy)-4-(dimethylamino)-6-methyltetrahydro-2H-pyran-3-yl acetate (10 mg, 0.024 mmol, 64%) after column chromatography (SiO₂, ethyl acetate) as a white solid.

2-(Cyclotetradecyloxy)-4-(dimethylamino)-6-methyltetrahydro-2H-pyran-3-01 (10). Following the general procedure, 2-(cyclotetradecyloxy)-4-(dimethylamino)-6-methyltetrahydro-2H-pyran-3-yl acetate (8 mg, 0.019 mmol), K₂CO₃ (15 mg, 0.11 mmol), and methanol (2 ml) were employed to afford 2-(cyclotetradecyloxy)-4-(dimethylamino)-6-methyltetrahydro-2H-pyran-3-ol (6 mg, 0.016 mmol, 86% yield) as a white solid.

2-(Cyclopentadecyloxy)-4-(dimethylamino)-6-methyltetrahydro-2H-pyran-3-yl acetate. Following the general procedure, cyclopentadecanol (35 mg, 0.15 mmol), 4-(dimethylamino)-2-fluoro-6-methyltetrahydro-2H-pyran-3-yl acetate (22 mg, 0.10 mmol), molecular sieves (200 mg) and BF₃.OEt₂ (20 μL, 0.20 mmol) were employed to obtain 2-(cyclopentadecyloxy)-4-(dimethylamino)-6-methyltetrahydro-2H-pyran-3-yl acetate (19 mg, 0.045 mmol, 45%) after column chromatography (SiO₂, ethyl acetate) as a white solid.

2-(Cyclopentadecyloxy)-4-(dimethylamino)-6-methyltetrahydro-2H-pyran-3-01 (12). Following the general procedure, 2-(cyclopentadecyloxy)-4-(dimethylamino)-6-methyltetrahydro-2H-pyran-3-yl acetate (10 mg, 0.023 mmol), K₂CO₃ (15 mg, 0.10 mmol), and methanol (2 ml) were employed to afford 2-(cyclopentadecyloxy)-4-(dimethylamino)-6-methyltetrahydro-2H-pyran-3-ol (8 mg, 0.021 mmol, 91% yield) as a white solid.

2-(Cyclohexyloxy)-4-(dimethylamino)-6-methyltetrahydro-2H-pyran-3-yl acetate. Following the general procedure, cyclohexanol (6 mg, 0.057 mmol), 4-(dimethylamino)-2-fluoro-6-methyltetrahydro-2H-pyran-3-yl acetate (22 mg, 0.10 mmol), molecular sieves (100 mg) and BF₃.OEt₂ (20 μL, 0.20 mmol) were employed to obtain 2-(cyclohexyloxy)-4-(dimethylamino)-6-methyltetrahydro-2H-pyran-3-yl acetate (10 mg, 0.033 mmol, 59%) after column chromatography (SiO₂, ethyl acetate) as a white solid. Spectral data matches that previously reported (Redlich, H.; Roy, W. Liebigs Ann. Chem. 1981, 1215).

2-(Cyclohexyloxy)-4-(dimethylamino)-6-methyltetrahydro-2H-pyran-3-ol (13). Following the general procedure, 2-(cyclohexyloxy)-4-(dimethylamino)-6-methyltetrahydro-2H-pyran-3-yl acetate (9 mg, 0.03 mmol), K₂CO₃ (16 mg, 0.12 mmol), and methanol were employed to afford 2-(cyclohexyloxy)-4-(dimethylamino)-6-methyltetrahydro-2H-pyran-3-ol (7 mg, 0.027 mmol, 90% yield) as a white solid.

2-(Decahydronaphthalen-1-yloxy)-4-(dimethylamino)-6-methyltetrahydro-2H-pyran-3-yl acetate. Following the general procedure, (±)-decahydronaphthalen-1-ol (16 mg, 0.10 mmol), 4-(dimethylamino)-2-fluoro-6-methyltetrahydro-2H-pyran-3-yl acetate (22 mg, 0.10 mmol), molecular sieves (100 mg) and BF₃.OEt₂ (20 μL, 0.20 mmol) were employed to obtain 2-(decahydronaphthalen-1-yloxy)-4-(dimethylamino)-6-methyltetrahydro-2H-pyran-3-yl acetate (20 mg, 0.58 mmol, 58%) after column chromatography (SiO₂, ethyl acetate) as a white solid.

2-(Decahydronaphthalen-1-yloxy)-4-(dimethylamino)-6-methyltetrahydro-2H-pyran-3-ol (14). Following the general procedure, 2-(decahydronaphthalen-1-yloxy)-4-(dimethylamino)-6-methyltetrahydro-2H-pyran-3-yl acetate (15 mg, 0.042 mmol), K₂CO₃ (30 mg, 0.22 mmol), and methanol (2 ml) were employed to afford 1:1 mixture of diastereomers 2-(decahydronaphthalen-1-yloxy)-4-(dimethylamino)-6-methyltetrahydro-2H-pyran-3-ol (12 mg, 0.038 mmol, 92% yield) as a white solid.

(2S,3R,4S,6R)-2-((3S,8R,9S,10R,13S,14S)-10,13-dimethyl-17-oxo-2,3,4,7,8,9,10,11,12,13,14,15,16,17-tetradecahydro-1H-cyclopenta[α]phenanthren-3-yloxy)-4-(dimethylamino)-6-methyltetrahydro-2H-pyran-3-yl acetate. Following the general procedure, trans-dehydroandrosterone (57 mg, 0.26 mmol), 4-(dimethylamino)-2-fluoro-6-methyltetrahydro-2H-pyran-3-yl acetate (57 mg, 0.26 mmol), molecular sieves (100 mg), and BF₃.OEt₂ (110 μL, 0.87 mmol) were employed to obtain (2S,3R,4S,6R)-2-((3S,8R,9S,10R,13S,14S)-10,13-dimethyl-17-oxo-2,3,4,7,8,9,10,11,12,13,14,15,16,17-tetradecahydro-1H-cyclopenta[α]phenanthren-3-yloxy)-4-(dimethylamino)-6-methyltetrahydro-2H-pyran-3-yl acetate (19 mg, 38%) as a white solid after column chromatography (1:19 methanol:ethyl acetate).

(3S,8R,9S,10R,13S,14S)-3-((2S,3R,4S,6R)-4-(dimethylamino)-3-hydroxy-6-methyltetrahydro-2H-pyran-2-yloxy)-10,13-dimethyl-3,4,7,8,9,10,11,12,13,14,15,16-dodecahydro-1H-cyclopenta[α]phenanthren-17(2H)-one (15). Following the general procedure, (2S,3R,4S,6R)-2-(3S,8R,9S,10R,13S,14S)-10,13-dimethyl-17-oxo-2,3,4,7,8,9,10,11,12,13,14,15,16,17-tetradecahydro-1H-cyclopenta[α]phenanthren-3-yloxy)-4-(dimethylamino)-6-methyltetrahydro-2H-pyran-3-yl acetate (11 mg, 0.022 mmol), K₂CO₃ (9 mg, 0.066 mmol), and methanol (3 mL) were employed to obtain (3S,8R,9S,10R,13S,14S)-3-((2S,3R,4S,6R)-4-(dimethylamino)-3-hydroxy-6-methyltetrahydro-2H-pyran-2-yloxy)-10,13-dimethyl-3,4,7,8,9,10,11,12,13,14,15,16-dodecahydro-1H-cyclopenta[α]phenanthren-17(2H)-one (14 mg, quantitative) as a white solid.

4-(Dimethylamino)-2-(hept-2-ynyloxy)-6-methyltetrahydro-2H-pyran-3-ol (16). Following the general procedure, 2-heptynol (6 mg, 0.057 mmol), 4-(dimethylamino)-2-fluoro-6-methyltetrahydro-2H-pyran-3-yl acetate (22 mg, 0.10 mmol), molecular sieves (100 mg) and BF₃.OEt₂ (20 μL, 0.20 mmol) were employed to obtain 4-(dimethylamino)-2-(hept-2-ynyloxy)-6-methyltetrahydro-2H-pyran-3-ol (9 mg, 0.033 mmol, 58%) after column chromatography (SiO₂, 1:19 methanol:CH₂Cl₂) as a white solid (acetate hydrolysis occurred during workup and purification).

2-(Dec-3-ynyloxy)-4-(dimethylamino)-6-methyltetrahydro-2H-pyran-3-yl acetate. Following the general procedure, 3-decynol (9 mg, 0.057 mmol), 4-(dimethylamino)-2-fluoro-6-methyltetrahydro-2H-pyran-3-yl acetate (22 mg, 0.10 mmol), molecular sieves (100 mg) and BF₃.OEt₂ (20 μL, 0.20 mmol) were employed to obtain 2-(dec-3-ynyloxy)-4-(dimethylamino)-6-methyltetrahydro-2H-pyran-3-yl acetate (13 mg, 0.037 mmol, 65%) after column chromatography (SiO₂, ethyl acetate) as a white solid.

2-(Dec-3-ynyloxy)-4-(dimethylamino)-6-methyltetrahydro-2H-pyran-3-ol (17). Following the general procedure, 2-(dec-3-ynyloxy)-4-(dimethylamino)-6-methyltetrahydro-2H-pyran-3-yl acetate (11 mg, 0.031 mmol), K₂CO₃ (20 mg, 0.14 mmol), and methanol were employed to afford 2-(dec-3-ynyloxy)-4-(dimethylamino)-6-methyltetrahydro-2H-pyran-3-ol (9 mg, 0.029 mmol, 94% yield) as a white solid.

4-(Dimethylamino)-6-methyl-2-(undec-lO-ynyloxy)tetrahydro-2H-pyran-3-yl acetate Following the general procedure, 10-undecynol (10 mg, 0.057 mmol), 4-(dimethylamino)-2-fluoro-6-methyltetrahydro-2H-pyran-3-yl acetate (22 mg, 0.10 mmol), molecular sieves (100 mg) and BF₃.OEt₂ (20 μL, 0.20 mmol) were employed to obtain 4-(dimethylamino)-6-methyl-2-(undec-10-ynyloxy)tetrahydro-2H-pyran-3-yl acetate (11 mg, 0.03 mmol, 52%) after column chromatography (SiO₂, ethyl acetate) as a white solid.

4-(Dimethylamino)-6-methyl-2-(undec-l O-ynyloxy)-tetrahydro-2H-pyran-3-ol (18). Following the general procedure, 4-(dimethylamino)-6-methyl-2-(undec-10-ynyloxy)tetrahydro-2H-pyran-3-yl acetate (7 mg, 0.019 mmol), K₂CO₃ (12 mg, 0.09 mmol), and methanol were employed to afford 4-(dimethylamino)-6-methyl-2-(undec-10-ynyloxy)-tetrahydro-2H-pyran-3-ol (6 mg, 0.018 mmol, 95% yield) as a white solid.

4-(Dimethylamino)-6-methyl-2-(pyren-1-yloxy)tetrahydro-2H-pyran-3-yl acetate. Following the general procedure, 1-hydroxypyrene (20 mg, 0.091 mmol), 4-(dimethylamino)-2-fluoro-6-methyltetrahydro-2H-pyran-3-yl acetate (25 mg, 0.114 mmol), molecular sieves (200 mg) and BF₃.OEt₂ (25 μL, 0.25 mmol) were employed to obtain 4-(dimethylamino)-6-methyl-2-(pyren-1-yloxy)tetrahydro-2H-pyran-3-yl acetate (23 mg, 0.055 mmol, 61%) after column chromatography (SiO₂, ethyl acetate) as a white solid.

4-(Dimethyl amino)-6-methyl-2-(pyren-1-yloxy)tetrahydro-2H-pyran-3-ol (19). Following the general procedure, 4-(dimethyl amino)-6-methyl-2-(pyren-1-yloxy)tetrahydro-2H-pyran-3-yl acetate (23 mg, 0.055 mmol), K₂CO₃ (30 mg, 0.22 mmol), and methanol were employed to afford 4-(dimethyl amino)-6-methyl-2-(pyren-1-yloxy)tetrahydro-2H-pyran-3-ol (14 mg, 0.037 mmol, 68% yield) as a white solid.

12-(tert-Butyldimethylsilyloxy)-1-trimethylsilyl)dodec-1-yn-6-ol. Magnesium turnings (25 mg, 1.04 mmol) were added to an ether solution of (6-bromohexyloxy)(t-butyl)dimethylsilane (200 mg, 1.02 mmol, Aldrich) in an oven dried flask and stirred for 2 h. An ether solution (5 mL) of 6-(trimethylsilyl)hex-5-ynal (Harris, G. D.; Herr, R. J.; Weinreb, S. M. J. Org. Chem. 1993, 58, 5452) (160 mg, 0.952 mmol), was added to the reaction mixture at 0° C. and stirred for 2 h. The reaction was quenched with saturated ammonium chloride solution and extracted with ethyl acetate. The combined organic layers were concentrated to afford the alcohol 12-(tert-butyldimethylsilyloxy)-1-trimethylsilyl)dodec-1-yn-6-ol (216 mg, 0.562 mmol, 59%) as a white solid after column chromatography (SiO₂, 1:5 ethyl acetate/hexanes).

Dodec-11-yne-1,7-diol. A 1.0 M n-Bu₄NF solution (0.70 ml, 0.70 mmol) in THF was added to a solution of (E)-cyclotetradec-2-enol-12-(tert-butyldimethylsilyloxy)-1-trimethylsilyl)dodec-1-yn-6-ol (66 mg, 0.17 mmol) in 4 mL THF and stirred for 1.5 h at rt. The reaction mixture was concentrated and purified by column chromatography (SiO2, 1:5 EtOAc, hexanes) to afford dodec-11-yne-1,7-diol (29 mg, 0.146 mmol, 86%) as a white solid.

7-Hydroxydodec-11-ynal. To the stirring solution of dodec-11-yne-1,7-diol (40 mg, 0.20 mmol), a solution of TEMPO (3 mg, 0.02 mmol) and tetrabutylammonium chloride (5 mg, 0.02 mmol) in 2 mL CH₂Cl₂ was added. An aqueous solution (2 mL) of K₂CO₃ (0.05 M) and Na₂CO₃ (0.5 M) was added to the solution followed by the addition of N-chlorosuccinamide (40 mg, 0.3 mmol) and stirred for 3 h. The organic layer was separated and the aqueous layer was extracted twice with 10 mL CH₂Cl₂. The combined organic layers were dried with magnesium sulfate, filtered, concentrated, and purified by column chromatography (SiO₂, 1:3 ethyl acetate/hexanes) to afford 7-hydroxydodec-11-ynal (30 mg, 0.12 mmol, 76% yield) as a colorless oil.

(E)-7-Triethylsilyloxy)cyclododec-5-enol. Toluene (10 mL) was injected to a mixture of Ni(cod)₂ (8 mg, 0.03 mmol), IMes.HCl (10 mg, 0.03 mmol), and potassium tert-butoxide (4 mg, 0.03 mmol) at rt and was stirred for 10 min. An additional 20 mL toluene and triethylsilane (0.10 ml, 0.30 mmol) were added, followed by syringe drive addition of a 10 mL toluene solution of 7-hydroxydodec-11-ynal (30 mg, 0.15 mmol) over 2 h. The reaction mixture was then stirred in air for 1 h, concentrated, and purified by column chromatography (SiO₂, 1:20 EtOAc:hexanes) to afford (E)-7-triethysiloxy)cyclododec-5-enol (10 mg, 0.032 mol, 21%) as a white solid (1:1 mixture of diastereomers).

7-(Triethylsilyloxy)cyclododecanol. Following the procedure used to synthesize cyclotetradecanol, (E)-7-triethysiloxy)cyclododec-5-enol (10 mg, 0.032 mmol) PtO₂ (3 mg, 0.013 mol) and hydrogen gas were employed to afford 7-(triethylsilyloxy)cyclododecanol (9 mg, 0.028 mmol, 90%) as a white solid (1:1 mixture of diastereomers).

7-(Triethylsilyloxy)cyclododecyl acetate. To the solution of 7-(triethylsilyloxy)cyclododecanol (30 mg, 0.095 mmol) in 5 mL CH₂Cl₂, K₂CO₃ (20 mg) and 4-dimethylamino pyridine (2 mg) were added and stirred for 10 min. The reaction mixture was cooled to 0° C. and Ac₂O (100 μL, 1.05 mmol) was added and stirred for 2 h. The reaction mixture was passed through a small plug of silica gel and washed with dichloromethane to afford 7-(triethylsilyloxy)cyclododecyl acetate as a 1:1 mixture of diastereomers. The diastereomers were then separated by column chromatography (SiO₂, 1:15 ethyl acetate/hexanes). Relative stereochemistry of the two diastereomers was not determined.

7-Hydroxycyclododecyl acetate.

Diastereomer A: Following the procedure used to prepare dodec-11-yn-1,7-diol, 7-(triethylsilyloxy)cyclododecyl acetate (10 mg, 0.028 mmol) and 1 M n-Bu₄NF (0.12 ml, 0.12 mmol) were employed to afford 7-hydroxycyclododecyl acetate (6 mg, 0.025 mmol, 89%) as white solid.

Diastereomer B: Following the procedure used to prepare (E)-cyclotetradec-2-enol, 7-(triethylsilyloxy)cyclododecyl acetate (10 mg, 0.028 mmol) and 1 M n-Bu₄NF (0.12 ml, 0.12 mmol) were employed to afford 7-hydroxycyclododecyl acetate (6 mg, 0.025 mmol, 89%) as white solid.

7-((2S,3R,4S,6R)-3-acetoxy-4-(dimethylamino)-6-methyltetrahydro-2H-pyran-2-yloxy)cyclododecyl acetate.

Diastereomer A: Following the general procedure, 7-hydroxycyclododecyl acetate (6 mg, 0.025 mmol), 4-(dimethylamino)-2-fluoro-6-methyltetrahydro-2H-pyran-3-yl acetate (11 mg, 0.05 mmol), molecular sieves (100 mg) and BF₃.OEt₂ (10 μL, 0.10 mmol) were employed to obtain the glycoside 7-((2S,3R,4S,6R)-3-acetoxy-4-(dimethylamino)-6-methyltetrahydro-2H-pyran-2-yloxy)cyclododecyl acetate (8 mg, 0.018 mmol, 72%) after column chromatography (SiO₂, ethyl acetate) as white solid.

Diastereomer B: Following the general procedure, 7-hydroxycyclododecyl acetate (6 mg, 0.025 mmol), 4-(dimethylamino)-2-fluoro-6-methyltetrahydro-2H-pyran-3-yl acetate (11 mg, 0.05 mmol), molecular sieves (100 mg) and BF₃.OEt₂ (10 μL, 0.10 mmol) were employed to obtain the glycoside (7 mg, 0.016 mmol, 64%) after column chromatography (SiO₂, ethyl acetate) as white solid.

4-(Dimethylamino)-2-(7-hydroxycyclododecyloxy)-6-methyltetrahydro-2H-pyran-3-ol

Diastereomer A: Following the general procedure, diastereomer A of 7-((2S,3R,4S,6R)-3-acetoxy-4-(dimethylamino)-6-methyltetrahydro-2H-pyran-2-yloxy)cyclododecyl acetate (3 mg, 0.007 mmol), K₂CO₃ (10 mg, 0.072 mmol), and methanol were employed to afford diastereomer A of the product (2 mg, 0.056 mmol, 93% yield) as a white solid.

Diastereomer B: Following the general procedure, diastereomer B of 7-((2S,3R,4S,6R)-3-acetoxy-4-(dimethylamino)-6-methyltetrahydro-2H-pyran-2-yloxy)cyclododecyl acetate (3 mg, 0.007 mmol), K₂CO₃ (10 mg, 0.072 mmol), and methanol were employed to afford diastereomer B of the product (2 mg, 0.056 mmol, 93% yield) as a white solid.

12-(tert-Butyldimethylsiloxy)-1-(trimethylsilyl)dodec-1-yn-S-ol. Following the procedure used to synthesize 12-(tert-butyldimethylsilyloxy)-1-trimethylsilyl)dodec-1-yn-6-ol, (7-bromoheptyloxy)(tert-butyl)dimethylsilane (Iqbal, M.; Li, Y. F.; Evans, P. Tetrahedron 2004, 60, 2531) (600 mg, 1.94 mmol), 5-(trimethylsilyl)pent-4-ynal (Cruciani, P.; Stammler, R.; Aubert, C.; Malacria, M. J. Org. Chem. 1996, 61, 2699) (369 mg, 2.39 mmol) and magnesium (48 mg, 2.00 mmol) were employed to afford 12-(tert-butyldimethylsiloxy)-1-(trimethylsilyl)dodec-1-yn-5-ol (200 mg. 0.78 mmol, 40%) as a colorless oil.

12-Hydroxydodec-1-yn-5-yl acetate. Following the procedure used to prepare dodec-11-yn-1,7-diol, 12-(tert-butyldimethylsiloxy)-1-(trimethylsilyl)dodec-1-yn-5-yl acetate (180 mg, 0.42 mol) and 1 M n-Bu₄NF (2 mL, 2.00 mmol) were employed to afford 12-hydroxydodec-1-yn-5-yl acetate (70 mg, 0.29 mmol, 69%) as white solid.

{E)-6-{Triethylsiloxy)cyclododec-4-enyl acetate. The above alcohol was oxidized by the same procedure described for 7-hydroxydodec-11-ynal and was directly used in the next transformation. Following the procedure used to synthesize (E)-(cyclotetradec-2-enyloxy)triethylsilane, Ni(COD)₂ (14 mg, 0.05 mmol), IPr·HCl (22 mg, 0.05 mmol), potassium tert-butoxide (6 mg, 0.05 mmol), triethylsilane (0.1 ml, 0.5 mmol) and 12-oxododec-1-yn-5-yl acetate (60 mg, 0.25 mmol) were employed to afford (E)-6-(triethylsiloxy)cyclododec-4-enyl acetate as 5:1 a mixture of diastereomers which were separated as diastereomer A (36 mg, 0.102 mol, 41%) and diastereomer B (7 mg, 0.020, 8%) as a colorless oil with column chromatography (SiO₂, 1:15 ethyl acetate/hexanes). The 1,6-stereochemical relationship of the diastereomers was not established.

(E)-6-Hydroxycyclododec-4-enyl acetate.

Diastereomer A (Major isomer) Following the procedure used to prepare (E)-cyclotetradec-2-enol, (E)-6-(triethylsiloxy)cyclododec-4-enyl acetate (36 mg, 0.102 mol) and n-Bu₄NF (0.30 ml, 0.30 mmol) were employed to afford (E)-6-hydroxycyclododec-4-enyl acetate (24 mg, 0.10 mmol, 98%) as white solid.

Diastereomer B (Minor isomer) The identical procedure as described for diastereomer A was followed.

6-Hydroxycyclododecyl acetate.

Diastereomer A: Following the procedure used to synthesize cyclotetradecanol, (E)-6-hydroxycyclododec-4-enyl acetate (24 mg, 0.10 mmol) PtO₂ (5 mg, 0.02 mol) and hydrogen gas were employed to afford 6-hydroxycyclododecyl acetate (20 mg, 0.083 mmol, 83%) as a white solid.

Diastereomer B: The identical procedure as described for diastereomer A was followed.

6-((2S,3R,4S,6R)-3-Acetoxy-4-(dimethylamino)-6-methyltetrahydro-2H-pyran-2-yloxy)cyclododecyl acetate.

Diastereomer A Following the general procedure, 6-hydroxycyclododecyl acetate (7 mg, 0.029 mmol), 4-(dimethylamino)-2-fluoro-6-methyltetrahydro-2H-pyran-3-yl acetate (22 mg, 0.10 mmol), molecular sieves (100 mg) and BF₃.OEt₂ (20 μL, 0.20 mmol) were employed to obtain 6-((2S,3R,4S,6R)-3-acetoxy-4-(dimethylamino)-6-methyltetrahydro-2H-pyran-2-yloxy)cyclododecyl acetate (8 mg, 0.018 mmol, 62%) after column chromatography (SiO₂, ethyl acetate) as a white solid. Major isomer (obtained as a 1:1 mixture of two diastereomers). The same procedure was repeated for diastereomer B on 1-2 mg of material. The diastereomer B product (1:1 mixture of diastereomers) was not characterized and was carried through to the next step.

4-(Dimethylamino)-2-(6-hydroxycyclododecyloxy)-6-methyltetrahydro-2H-pyran-3-ol. The four diastereomers 7a, 7c, 7e, and 7g were obtained as one pair with the 1,6-trans relationship and one pair with the 1,6-cis relationship. The trans pair and cis pair were not stereochemically characterized, but all four compounds were distinguished by LCMS analysis.

Diastereomers A Following the general procedure, acetate 6-((2S,3R,4S,6R)-3-acetoxy-4-(dimethylamino)-6-methyltetrahydro-2H-pyran-2-yloxy)cyclododecyl acetate (3 mg, 0.007 mmol), K₂CO₃ (10 mg, 0.072 mmol), and methanol were employed to afford 4-(dimethylamino)-2-(6-hydroxycyclododecyloxy)-6-methyltetrahydro-2H-pyran-3-ol (2 mg, 0.0056 mmol, 80% yield) as a white solid. Major isomer (obtained as a 1:1 mixture of two diastereomers):

Diastereomers B The same procedure was repeated for diastereomer B on 1-2 mg of material. The diastereomer B product (1:1 mixture of diastereomers) obtained in trace amount was only characterized by LCMS analysis.

13-(tert-Butyldimethylsilyloxy)-1-(trimethylsilyl)tridec-1-yn-7-ol. Razor scraped Mg turnings (161 mg, 6.6 mmol) were added and a reflux condenser was attached. tert-Butyl(6-chlorohexyloxy)dimethylsilane (Kobierski, M. E.; Kim, S.; Murthi, K. K.; Iyer, R. S.; Saloman, R. G. J. Org. Chem. 1994, 59, 6044) (553 mg, 2.2 mmol) was added as a solution in THF (2 mL). Several drops of 1,2-dibromoethane were added and the reaction mixture was heated to reflux for 1 h. Several more drops of 1,2-dibromoethane were added. 7-(Trimethylsilyl)-hept-6-ynal (Synthesis of 6-heptyn-1-ol: Li, M. and O'Dougherty, G. A. Org. Lett. 2006, 8, 6087; Synthesis of 7-(trimethylsilyl)hept-6-yn-1-ol: Wu, G.; Cederbaum, F. E.; Negishi, E. Tetrahedron Lett. 1990, 31, 493; Synthesis of 7-(trimethylsilyl)hept-6-ynal: Shim, S.C.; Hwang, J.-T.; Kang, H.-Y.; Chang, M. H. Tetrahedron Lett. 1990, 31, 4765) (268 mg, 1.5 mmol) was then added dropwise over 20 min to the refluxing mixture as a solution in THF (2 mL). This was allowed to reflux overnight and was then removed from heat. The reaction mixture was quenched with saturated NH₄Cl and extracted with ethyl acetate. The combined organic layers were washed with brine, dried over MgSO₄, and filtered. Flash column chromatography (5% ethyl acetate/hexanes) afforded 13-(tert-butyldimethylsilyloxy)-1-(trimethylsilyl)tridec-1-yn-7-ol (245 mg, 42%) as a colorless oil.

Following the procedure used to synthesize 7-(triethylsilyloxy)cyclododecyl acetate, 13-(tert-butyldimethylsilyloxy)-1-(trimethylsilyl)tridec-1-yn-7-ol (200 mg, 0.50 mmol), K₂CO₃ (97 mg, 0.70 mmol), and 4-dimethylamino pyridine (6 mg, 0.050 mmol) were employed to obtain 13-(tert-butyldimethylsilyloxy)-1-(trimethylsilyl)tridec-1-yn-7-yl acetate (203 mg, 92%) as a clear oil after flash column chromatography (SiO₂, 5% ethyl acetate/hexanes).

13-Hydroxytridec-1-yn-7-yl acetate. Following the procedure used to prepare dodecy-11-yne-1,7-diol, 13-(tert-butyldimethylsilyloxy)-1-(trimethylsilyl)tridec-1-yn-7-yl acetate (190 mg, 0.43 mmol) and n-Bu₄NF (1 M in THF, 0.86 mL, 0.86 mmol) were employed to obtain 13-hydroxytridec-1-yn-7-yl acetate (109 mg, 99%) after flash column chromatography (SiO₂, 30% ethyl acetate/hexanes).

13-0xotridec-1-yn-7-yl acetate. To a stirring solution of 13-hydroxytridec-1-yn-7-yl acetate (100 mg, 0.39 mmol) in CH₂Cl₂ (3 mL) was added PCC (170 mg, 0.78 mmol). The reaction was allowed to stir for 4 h and was then filtered through a plug of silica gel to afford 13-oxotridec-1-yn-7-yl acetate (81 mg, 82%) after flash column chromatography (SiO₂, 25% ethyl acetate/hexanes).

8-(Triethylsilyloxy)cyclotridec-6-enyl acetate. A flame dried round bottom flask was charged with Ni(cod)₂ (5 mg, 0.018 mmol), IMes.HCl (6 mg, 0.018 mmol), and t-BuOK (2 mg, 0.018 mmol) in the glove box. To this was added THF (5 mL) and the catalyst was allowed to form for 10 min. The catalyst system was then diluted with THF (10 mL) and a solution of 13-oxotridec-1-yn-7-yl acetate (46 mg, 0.18 mmol) and Et₃SiH (58 μL, 0.36 mmol) in THF (15 mL) was added over 2 h via syringe pump. Another flask was charged with Ni(cod)₂ (5 mg, 0.018 mmol), IMes.HCl (6 mg, 0.018 mmol), and t-BuOK (2 mg, 0.018 mmol), THF (3 mL) was added, and the catalyst was allowed to form over 10 min. This second batch of catalyst was added dropwise to the first flask after addition of the ynal had completed. The reaction was stirred for 1 h, then opened to air and stirred for 30 min. The reaction mixture was poured through a plug column and then purified via flash column chromatography (SiO₂, 1.5% ethyl acetate/hexanes) to afford 8-(triethylsilyloxy)cyclotridec-6-enyl acetate as a 1:1 mixture of diastereomer A (16 mg, 24%) and diastereomer B (15 mg, 22%), both as colorless oils.

8-Hydroxycyclohex-6-enyl acetate.

Diastereomer A Following the procedure to make dodec-11-yne-1,7-diol, 8-(triethylsilyloxy)cyclotridec-6-enyl acetate (24 mg, 0.065 mmol) and n-Bu₄NF (1 M, 0.1 mL, 0.098 mmol) afforded 8-hydroxycyclohex-6-enyl acetate (15 mg, 91%) as a colorless oil after purification by flash column chromatography (SiO₂, 15% ethyl acetate/hexanes).

Diastereomer B Following the procedure to make dodec-11-yne-1,7-diol, 8-(triethylsilyloxy)cyclotridec-6-enyl acetate (23 mg, 0.061 mmol) and n-Bu₄NF (1 M, 0.1 mL, 0.092 mmol) afforded 8-hydroxycyclohex-6-enyl acetate (8 mg, 51%) as a colorless oil after purification by flash column chromatography (SiO₂, 15% ethyl acetate/hexanes).

7-Hydroxycyclotridecyl acetate.

Diastereomer A To a solution of 8-hydroxycyclohex-6-enyl acetate (15 mg, 0.059 mmol) in methanol (10 mL) was added 10% Pd/C (5 mg). The system was purged with a H₂ balloon for 20 min. The balloon was then refilled and the reaction was allowed to react at rt under a H₂ atmosphere overnight. The reaction mixture was run through a plug of silica gel and flushed with 1:1 ethyl acetate:hexanes to afford 7-hydroxycyclotridecyl acetate (6 mg, 40%) after flash column chromatography (15% ethyl acetate/hexanes).

Diastereomer B Following the procedure used for diastereomer A, 8-hydroxycyclohex-6-enyl acetate (8 mg, 0.031 mmol) and 10% Pd/C (5 mg) in methanol (10 mL) afforded 7-hydroxycyclotridecyl acetate (7 mg, 88%).

(2S,3R,4S,6R)-4-(dimethylamino)-6-methyl-2-(7-(triethylsilyloxy) cyclotridecyloxy)tetrahydro-2H-pyran-3-yl acetate.

Diastereomer A Following the general procedure, 7-hydroxycyclotridecyl acetate (6 mg, 0.023 mmol), (2S,3R,4S,6R)-4-(dimethylamino)-6-methyltetrahydro-2H-pyran-3-yl acetate (8 mg, 0.035 mmol), 4 Å molecular sieves (˜100 mg), and BF₃.OEt₂ (14 μL, 0.12 mmol) were employed to afford the product after column chromatography (SiO₂, 1% Et₃N/ethyl acetate). A trace amount of product (1-2 mg) was obtained and was carried on to the next step without characterization.

Diastereomer B Following the general procedure, 7-hydroxycyclotridecyl acetate (1 mg, 0.0039 mmol), (2S,3R,4S,6R)-4-(dimethylamino)-6-methyltetrahydro-2H-pyran-3-yl acetate (1 mg, 0.0046 mmol), 4 Å molecular sieves (˜20 mg), and BF₃.OEt₂ (3 μL, 0.024 mmol) were used to obtain the product, which was carried on to the next step without further purification.

(2S,3R,4S,6R)-4-(dimethylamino)-2-(7-hydroxycyclotridecyloxy)-6-methyltetrahydro-2H-pyran-3-ol. The four diastereomers were obtained as one pair with the 1,7-trans relationship and one pair with the 1,7-cis relationship. The pairs were not stereochemically characterized.

Diastereomers A Following the general procedure, (2S,3R,4S,6R)-4-(dimethylamino)-6-methyl-2-(7-(triethylsilyloxy)cyclotridecyloxy)tetrahydro-2H-pyran-3-yl acetate and K₂CO₃ were used to afford the crude product, which was analyzed without purification.

Diastereomers B Following the general procedure, (2S,3R,4S,6R)-4-(dimethylamino)-6-methyl-2-(7-(triethylsilyloxy)cyclotridecyloxy)tetrahydro-2H-pyran-3-yl acetate and K₂CO₃ were used to afford the crude product, which was analyzed without purification.

13-(tert-Butyldimethylsilyloxy)-1-(trimethylsilyl)tridec-1-yn-5-ol. Following the procedure used to synthesize 13-(tert-butyldimethylsilyloxy)-1-(trimethylsilyl)tridec-1-yn-7-ol, (8-bromooctyloxy)(tert-butyl)dimethylsilane (Nilewski, C; Geisser, R W; Carreira, E M. Nature. 2009, 457, 573; Ishigami, K.; Kato, T.; Akasaka, K.; Watanabe, H. Tetrahedron Lett. 2008, 49, 5077) (1.0 g, 3.1 mmol), Mg turnings (376 mg, 15.5 mmol), and 5-(trimethylsilyl)-pent-4-ynal (370 mg, 2.5 mmol) were employed to yield the product (577 mg, 60%) as a colorless oil after flash column chromatography (SiO₂, 5% ethyl acetate/hexanes).

13-(tert-Butyldimethylsilyloxy)-1-(trimethylsilyl)tridec-1-yn-5-yl acetate. Following the procedure used to synthesize 7-(triethylsilyloxy)cyclododecyl acetate, 13-(tert-butyldimethylsilyloxy)-1-(trimethylsilyl)tridec-1-yn-5-ol (219 mg, 0.55 mmol), acetic anhydride (0.5 mL, 5.5 mmol), K₂CO₃ (106 mg, 0.77 mmol), and 4-dimethylamino pyridine (7 mg, 0.06 mmol) were employed to obtain the product (227 mg, 94%) as a colorless oil after flash column chromatography (SiO₂, 5% ethyl acetate/hexanes).

13-hydroxytridec-1-yn-5-yl acetate. Following the procedure used to prepare dodec-11-yne-1,7-diol, 13-(tert-butyldimethylsilyloxy)-1-(trimethylsilyl)tridec-1-yn-5-yl acetate (227 mg, 0.51 mmol) and 1 M n-Bu₄NF (1.03 mL, 1.03 mmol) were employed to obtain 13-hydroxytridec-1-yn-5-yl acetate (128 mg, 98%) as a light yellow oil after column chromatography (SiO₂, 40% ethyl acetate/hexanes).

13-oxotridec-1-yn-5-yl acetate. Following the procedure used to synthesize 13-oxotridec-1-yl-7-yl acetate, 13-hydroxytridec-1-yn-5-yl acetate (102 mg, 0.4 mmol) and PCC (173 mg, 0.8 mmol) were employed to yield the product (84 mg, 83%) as a colorless oil after flash column chromatography (SiO₂, 40% ethyl acetate/hexanes).

(E)-6-(triethylsilyloxy)cyclotridec-4-enyl acetate. Using the procedure to synthesize 8-(triethylsilyloxy)cyclotridec-6-enyl acetate, 13-oxotridec-1-yn-5-yl acetate (150 mg, 0.59 mmol), Ni(cod)₂ (16 mg, 0.06 mmol), IMes.HCl (20 mg, 0.06 mmol), t-BuOK (7 mg, 0.06 mmol), and triethylsilane (190 μL, 1.2 mmol) were employed to yield a 2.7:1 mixture of diastereomers, which were separated by column chromatography (1.5% ethyl acetate/hexanes) to obtain diastereomer A (27 mg, 17%) and diastereomer B (10 mg, 7%), both as colorless oils. The 1,6-stereochemical relationship of the diastereomers was not established.

(E)-6-hydroxycyclotridec-4-enyl acetate.

Diastereomer A (major diastereomer) Following the procedure used to synthesize dodec-11-yne-1,7-diol, (E)-6-(triethylsilyloxy)cyclotridec-4-enyl acetate (27 mg, 0.07 mmol) and 1 M n-Bu₄NF (80 μL, 0.08 mmol) were employed to afford (E)-6-hydroxycyclotridec-4-enyl acetate (16 mg, 84%) as a clear oil.

Diastereomer B (minor diastereomer) Following the procedure used to synthesize (E)-cyclotetradec-2-enol, (E)-6-(triethylsilyloxy)cyclotridec-4-enyl acetate (23 mg, 0.06 mmol) and 1 M n-Bu₄NF (90 μL, 0.09 mmol) were employed to afford (E)-6-hydroxycyclotridec-4-enyl acetate (16 mg, 99%) as a clear oil.

6-hydroxycyclotridecyl acetate.

Diastereomer A (major diastereomer) Following the procedure used to synthesize 7-hydroxycyclotridecyl acetate, (E)-6-hydroxycyclotridec-4-enyl acetate (16 mg, 0.06 mmol), Pd/C (10% Pd) and hydrogen gas were employed to afford 6-hydroxycyclotridecyl acetate (10 mg, 63%) as a colorless oil.

Diastereomer B (minor diastereomer) Following the procedure used to synthesize 7-hydroxycyclotridecyl acetate, (E)-6-hydroxycyclotridec-4-enyl acetate (15 mg, 0.06 mmol), Pd/C (10% Pd) and hydrogen gas were employed to afford 6-hydroxycyclotridecyl acetate (8 mg, 53%) as a colorless oil.

6-((2S,3R,4S,6R)-3-acetoxy-4-(dimethylamino)-6-methyltetrahydro-2H-pyran-2-yloxy)cyclotridecyl acetate.

Diastereomer A (major diastereomer) Following the general procedure, 7-hydroxycyclotridecyl acetate (10 mg, 0.04 mmol), (2S,3R,4S,6R)-4-(dimethylamino)-6-methyltetrahydro-2H-pyran-3-yl acetate (19 mg, 0.09 mmol), 4 Å MS (100 mg), and BF₃.OEt₂ (24 μL, 0.20 mmol) were employed to obtain the product (11 mg, 61%, as a mixture of two diastereomers) after flash column chromatography (SiO₂, 1% NEt₃, 5% MeOH/ethyl acetate).

Diastereomer B (minor diastereomer) Following the general procedure, 7-hydroxycyclotridecyl acetate (5 mg, 0.02 mmol), (2S,3R,4S,6R)-4-(dimethylamino)-6-methyltetrahydro-2H-pyran-3-yl acetate (8 mg, 0.04 mmol), 4 Å MS (100 mg), and BF₃.OEt₂ (12 μL, 0.09 mmol) were employed to obtain the product (2.5 mg, 29%, as a mixture of two diastereomers) after flash column chromatography (SiO₂, 1% NEt₃, 5% MeOH/ethyl acetate).

(2S,3R,4S,6R)-4-(dimethylamino)-2-(6-hydroxycyclotridecyloxy)-6-methyltetrahydro-2H-pyran-3-ol.

Diastereomers A (major) Following the general procedure, the diastereomeric mixture of 6-((2S,3R,4S,6R)-3-acetoxy-4-(dimethylamino)-6-methyltetrahydro-2H-pyran-2-yloxy)cyclotridecyl acetate (10 mg, 0.02 mmol) and K₂CO₃ (13 mg, 0.09 mmol) were used to afford the product (6 mg, 71%, as a mixture of two diastereomers) as a pale yellow solid.

Diastereomers B (minor) Following the general procedure, the diastereomeric mixture of 6-((2S,3R,4S,6R)-3-acetoxy-4-(dimethylamino)-6-methyltetrahydro-2H-pyran-2-yloxy)cyclotridecyl acetate (2.5 mg, 0.005 mmol) and K₂CO₃ (4 mg, 0.03 mmol) were used to afford the product (2 mg, 99%, as a mixture of two diastereomers).

Synthesis of authentic propargyl alcohol: 11-(tert-butyldimethylsilyloxy)-1-(trimethylsilyl)undec-1-yn-3-yl acetate. Following the procedure used to synthesize 7-(triethylsilyloxy)cyclododecyl acetate, 5-(tert-butyldimethylsilyloxy)-1-(trimethylsilyl)pent-1-yn-3-ol^{xvi} (77 mg, 0.21 mmol), acetic anhydride (0.2 mL, 2.1 mmol), K₂CO₃ (40 mg, 0.29 mmol), and DMAP (3 mg, 0.02 mmol) were employed to obtain the product (80 mg, 93%) as a pale yellow oil after flash column chromatography (SiO₂, 5% ethyl acetate/hexanes).

5-hydroxypent-1-yn-3-yl acetate. Following the procedure used to prepare dodec-11-yne-1,7-diol, 11-(tert-butyldimethylsilyloxy)-1-(trimethylsilyl)undec-1-yn-3-yl acetate (72 mg, 0.17 mmol) and 1 M TBAF (0.35 mL, 0.35 mmol) were employed to obtain 5-hydroxypent-1-yn-3-yl acetate (37 mg, 95%) as a light yellow oil after column chromatography (SiO₂, 20% ethyl acetate/hexanes).

5-<<2R,3R,4S,6R)-3-acetoxy-4-(dimethylamino)-6-methyltetrahydro-2H-pyran-2-yloxy)pent-1-yn-3-yl acetate. Following the general procedure, 5-hydroxypent-1-yn-3-yl acetate (31 mg, 0.14 mmol), (2S,3R,4S,6R)-4-(dimethylamino)-6-methyltetrahydro-2H-pyran-3-yl acetate (45 mg, 0.21 mmol), 4 Å molecular sieves (˜150 mg), and BF₃.OEt₂ (85 μL, 0.68 mmol) were employed to afford the product (13 mg, 22%) after column chromatography (SiO₂, ethyl acetate).

(2R,3R,4S,6R)-4-(dimethylamino)-2-(3-hydroxypent-4-ynyloxy)-6-methyltetrahydro-2H-pyran-3-ol. Following the general procedure, 5-((2R,3R,4S,6R)-3-acetoxy-4-(dimethylamino)-6-methyltetrahydro-2H-pyran-2-yloxy)pent-1-yn-3-yl acetate (6 mg, 0.014 mmol) and K₂CO₃ (10 mg, 0.070 mmol) were employed to afford the product (5 mg, quantitative).

All of the above U.S. patents, U.S. patent application publications, U.S. patent applications, foreign patents, foreign patent applications and non patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

1. A method of hydroxylating or epoxidating a substrate comprising contacting the substrate with a cytochrome P450 enzyme to form a hydroxylated or epoxidized product, wherein the substrate comprises a desosaminyl, a 1,2-diol-3-dimethylaminocyclohexane, a 1,2-diol-3-methylaminocyclohexane, or an N,N-dimethylaminopropylether moiety.

2. The method of claim 1, wherein the cytochrome P450 enzyme is a bacterial biosynthetic cytochrome P450, a mitochondrial cytochrome P450, or a eukaryotic cytochrome P450.

3. (canceled)

4. (canceled)

5. The method of claim 1, wherein the cytochrome P450 enzyme is PikC or is selected from the group consisting of EryF, MycG, TylI, TylHI, and TamI.

6. (canceled)

7. The method of claim 1, wherein the cytochrome P450 enzyme is mutated.

8. The method of claim 1, wherein the cytochrome P450 enzyme is mutated PikC.

9. (canceled)

10. The method of claim 1, wherein the P450 enzyme is fused to a heterologous reductase domain.

11. The method of claim 10, wherein the reductase domain comprises RhFRED from Rhodococcus sp. NCIMB 9784.

12. (canceled)

13. The method of claim 1, wherein the desosaminyl, 1,2-diol-3-dimethylaminocyclohexane, 1,2-diol-3-methylaminocyclohexane, or N,N-dimethylaminopropylether moiety is covalently attached to the substrate through an acetal bond, an ester bond, an ether bond, a ketal bond, a peptide bond, a carbon-carbon bond, a hemi-acetal bond, or a hemi-ketal bond.

14. (canceled)

15. (canceled)

16. (canceled)

17. (canceled)

18. (canceled)

19. (canceled)

20. A P450 substrate modified with a desosaminyl, 1,2-diol-3-dimethylaminocyclohexane, 1,2-diol-3-methylaminocyclohexane, or N,N-dimethylaminopropylether moiety, wherein P450 does not act or is less active on the substrate without the desosaminyl, 1,2-diol-3-dimethylaminocyclohexane, 1,2-diol-3-methylaminocyclohexane, or N,N-dimethylaminopropylether moiety.

21. The substrate of claim 20, wherein the substrate comprises a polyketide, a macrolide, a cycloalkane, an aromatic compound, a heteroaromatic compound, or a steroid.

22. The substrate of claim 20, wherein the substrate is modified with desosaminyl moiety at a C-1 position in the desosaminyl moiety.

23. The substrate of claim 20, wherein the desosaminyl, 1,2-diol-3-dimethylaminocyclohexane, 1,2-diol-3-methylaminocyclohexane, or N,N-dimethylaminopropylether moiety is covalently attached to the substrate through an acetal bond, an ester bond, an ether bond, a ketal bond, a peptide bond, a carbon-carbon bond, a hemi-acetal bond, or a hemi-ketal bond.

24. A chimeric protein comprising a mutant cytochrome P450 and a heterologous reductase domain wherein the chimeric protein has self-sufficiency and increased catalytic efficiency compared to native P450 monooxygenases, said chimeric protein having the activity of catalyzing C—H bond oxidation of a substrate modified with a desosaminyl, 1,2-diol-3-dimethylaminocyclohexane, 1,2-diol-3-methylaminocyclohexane, or N,N-dimethylaminopropylether moiety.

25. The chimeric protein of claim 24, wherein said substrate is a polyketide, a cycloalkane, an aromatic molecule, a heteroaromatic molecule, an alkyne, or a steroidyl molecule.

26. The chimeric protein of claim 24, wherein the C—H oxidization comprises oxidation of one or more primary, secondary or tertiary carbon atoms of said substrate.

27. (canceled)

28. (canceled)

29. The chimeric protein of claim 24, wherein said mutant cytochrome P450 is a mutant eukaryotic cytochrome P450.

30. The chimeric protein of claim 24, wherein said mutant cytochrome P450 is PikC_{D50N}.

31. The chimeric protein of claim 24, wherein said mutant cytochrome P450 is selected from the group consisting of a mutant PikC, a mutant EryF, a mutant MycG, a mutant TylI, a mutant TylHI, and a mutant TamI.

32. (canceled)

33. (canceled)

34. A compound selected from the group consisting of 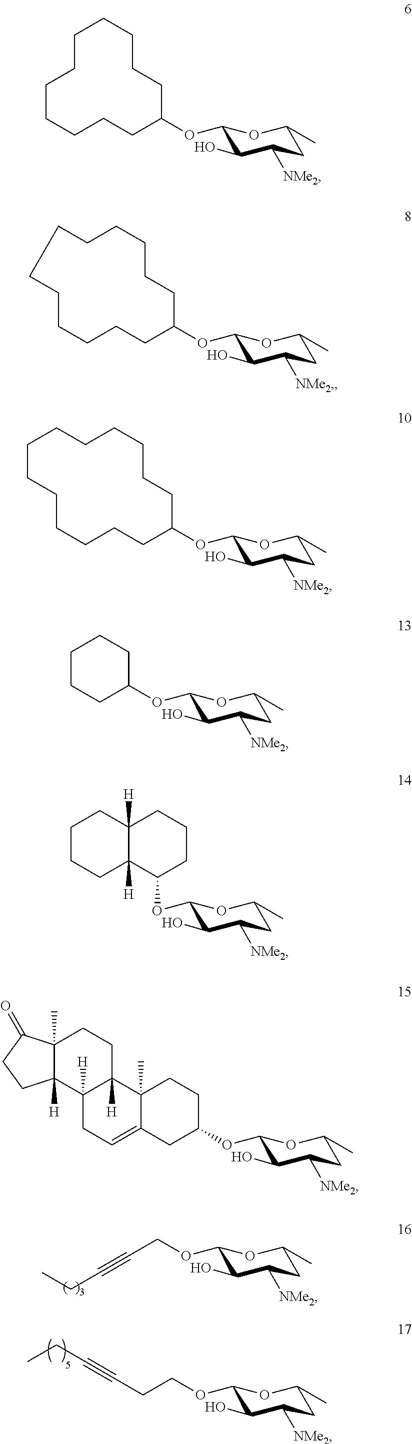 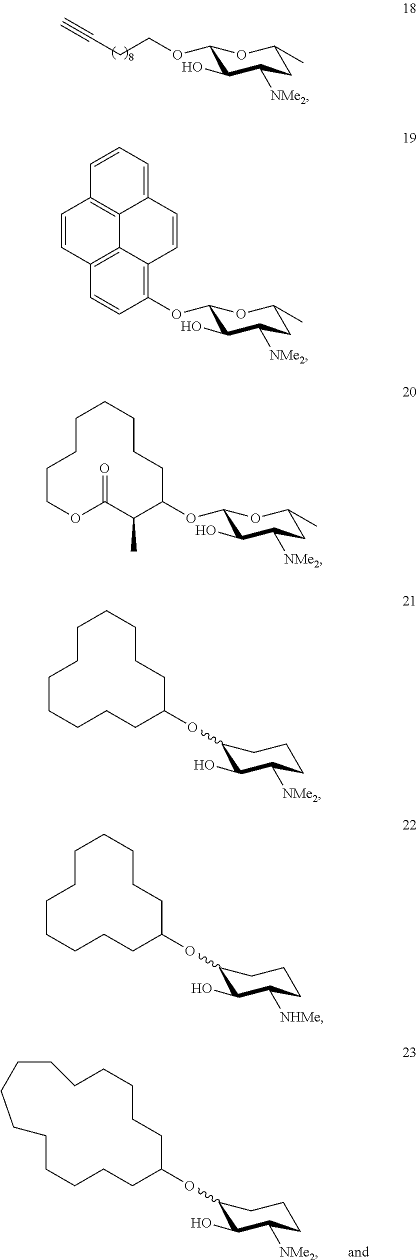 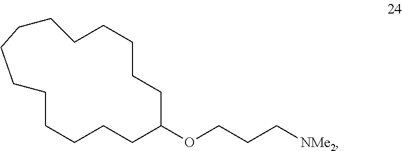
or salt thereof.

35. A method of inhibiting a bacterial infection comprising contacting a cell with a substrate of claim 20 or a compound of claim 34.

36. (canceled)

37. (canceled)

38. (canceled)
